SEQUENCE LISTING (117 sequences)

SEQ ID NO: 1 - DNA, 116 bp, Escherichia coli
  source 1..116: mol_type = genomic DNA
ttggtacgtaaacgcatcgtggggctatagctcagctgggagagcgcttgcatggcatgcaagaggtcagcggttcgatcccgcttagctccaccaaatttccaaccctcgctgca

SEQ ID NO: 2 - DNA, 114 bp, Escherichia coli
  source 1..114: mol_type = genomic DNA
ctgaaaggcctgaagaatttggcgcgttaacaaagcggttatgtagcggattgcaaatccgtctagtccggttcgactccggaacgcgcctccactttcttcccgagcccggat

SEQ ID NO: 3 - DNA, 117 bp, Escherichia coli
  source 1..117: mol_type = genomic DNA
tggttgtaaaagaattcggtggagcggtagttcagtcggttagaatacctgcctgtcacgcagggggtcgcgggttcgagtcccgtccgttccgccacttattaagaagcctcgagt

SEQ ID NO: 4 - DNA, 116 bp, Escherichia coli
  source 1..116: mol_type = genomic DNA
gcgtgataagcaattttcgtgtccccttcgtctagaggcccaggacaccgccctttcacggcggtaacaggggttcgaatcccctaggggacgccacttgctggtttgtgagtgaa

SEQ ID NO: 5 - DNA, 116 bp, Escherichia coli
  source 1..116: mol_type = genomic DNA
caggtttaatgcgccccgttgcccggatagctcagtcggtagagcaggggattgaaaatccccgtgtccttggttcgattccgagtccgggcaccactaattcttaagaacccgcc

SEQ ID NO: 6 - DNA, 114 bp, Escherichia coli
  source 1..114: mol_type = genomic DNA
aatgccttacgcatctcgaagcgggcgtagttcaatggtagaacgagagcttcccaagctctatacgagggttcgattcccttcgcccgctccaatttatcttcgccgtaatac

SEQ ID NO: 7 - DNA, 116 bp, Escherichia coli
  source 1..116: mol_type = genomic DNA
gcgtaacaagatttgtagtggtggctatagctcagttggtagagccctggattgtgattccagttgtcgtgggttcgaatcccattagccaccccattattagaagttgtgacaat

SEQ ID NO: 8 - DNA, 107 bp, Escherichia coli
  source 1..107: mol_type = genomic DNA
atgagcagtaaaacctctacaggcttgtagctcaggtggttagagcgcacccctgataagggtgaggtcggtggttcaagtccactcaggcctaccaaatttgcacg

SEQ ID NO: 9 - DNA, 163 bp, Escherichia coli
  source 1..163: mol_type = genomic DNA
ctgaatgattaaggcagcataatcccgcaaggggtcgttagctcagttggtagagcagttgacttttaatcaattggtcgcaggttcgaatcctgcacgacccaccaatgtaaaaaagcgccctaaaggcgcttttttgctatctgcgatactcaaagattcg

SEQ ID NO: 10 - DNA, 125 bp, Escherichia coli
  source 1..125: mol_type = genomic DNA
tttccgcatacctcttcagtgccgaagtggcgaaatcggtagacgcagttgattcaaaatcaaccgtagaaatacgtgccggttcgagtccggccttcggcaccaaaagtatgtaaatagacctc

SEQ ID NO: 11 - DNA, 135 bp, Escherichia coli
  source 1..135: mol_type = genomic DNA
ccccagccatcgaagaaacaatctggctacgtagctcagttggttagagcacatcactcataatgatggggtcacaggttcgaatcccgtcgtagccaccaaattctgaatgtatcgaatatgttcggcaaattc

SEQ ID NO: 12 - DNA, 117 bp, Escherichia coli
  source 1..117: mol_type = genomic DNA
gtatagtgcgcatccacggacgcggggtggagcagcctggtagctcgtcgggctcataacccgaaggtcgtcggttcaaatccggcccccgcaaccaattaaaatttgatgaagtaa

SEQ ID NO: 13 - DNA, 116 bp, Escherichia coli
  source 1..116: mol_type = genomic DNA
attcgccccgttcacacgattcctctgtagttcagtcggtagaacggcggactgttaatccgtatgtcactggttcgagtccagtcagaggagccaaattctaaaaattcgctttt

SEQ ID NO: 14 - DNA, 117 bp, Escherichia coli
  source 1..117: mol_type = genomic DNA
agtaagatgcgccccgcattcggtgattggcgcagcctggtagcgcacttcgttcgggacgaaggggtcggaggttcgaatcctctatcaccgaccaaattcgaaaagcctgctcaa

SEQ ID NO: 15 - DNA, 115 bp, Escherichia coli
  source 1..115: mol_type = genomic DNA
accttgtaagtgcacccagttggggtatcgccaagcggtaaggcaccggattctgattccggcattccgaggttcgaatcctcgtaccccagccacattaaaaaagctcgcttcg

SEQ ID NO: 16 - DNA, 117 bp, Escherichia coli
  source 1..117: mol_type = genomic DNA
gtagaataagttttcccgatgcatccgtagttcagctggatagagtactcggctacgaaccgagcggtcggaggttcgaatcctcccggatgcaccatattctccgtaaccttcagc

SEQ ID NO: 17 - DNA, 130 bp, Escherichia coli
  source 1..130: mol_type = genomic DNA
atcatggcaaccatctgaacggagagatgccggagcggctgaacggaccggtctcgaaaaccggagtaggggcaactctaccgggggttcaaatccccctctctccgccactttatcaatgacttatctc

SEQ ID NO: 18 - DNA, 116 bp, Escherichia coli
  source 1..116: mol_type = genomic DNA
cgtgaacatgtcctttcagggccgatatagctcagttggtagagcagcgcattcgtaatgcgaaggtcgtaggttcgactcctattatcggcaccattaaaatcaaattgttacgt

SEQ ID NO: 19 - DNA, 117 bp, Escherichia coli
  source 1..117: mol_type = genomic DNA
ccaattgaacgcaccatcctgcgtccgtagctcagttggttagagcaccaccttgacatggtgggggtcggtggttcgagtccactcggacgcaccagattttcttaatctggtctt

SEQ ID NO: 20 - DNA, 133 bp, Escherichia coli
  source 1..133: mol_type = genomic DNA
cgcgggttcgagtcccgtccgttccgccaccctaattaggggcgtagttcaattggtagagcaccggtctccaaaaccgggtgttgggagttcgagtctctccgcccctgccagaaatcatccttagcgaaag

SEQ ID NO: 21 - DNA, 123 bp, Escherichia coli
  source 1..123: mol_type = genomic DNA
tagtcggcaccatcaagtccggtggggttcccgagcggccaaagggagcagactgtaaatctgccgtcacagacttcgaaggttcgaatccttcccccaccaccaatttcggccacgcgatgg

SEQ ID NO: 22 - DNA, 458 bp, Escherichia coli
  source 1..458: mol_type = genomic DNA
ctctccctataatgcgactccacacagcgggggtgattagctcagctgggagagcacctcccttacaaggagggggtcggcggttcgatcccgtcatcacccaccaactactttatgtagtctccgccgtgtagcaagaaattgagaagtgggtgattagctcagctgggagagcacctcccttacaaggagggggtcggcggttcgatcccgtcatcacccaccactttctcgccagctaaatttcttgtaaaaatgtgaagtaccgaagtgggtgattagctcagctgggagagcacctcccttacaaggagggggtcggcggttcgatcccgtcatcacccaccacttcgggtcgttagctcagttggtagagcagttgacttttaatcaattggtcgcaggttcgaatcctgcacgacccaccaatgtaaaaaagcgccctaaaggcgcttttt

SEQ ID NO: 23 - DNA, 1159 bp, Escherichia coli
  source 1..1159: mol_type = genomic DNA
tatcaaacaaaccgaaagcaacgaaaaagtgggtcgttagctcagttggtagagcagttgacttttaatcaattggtcgcaggttcgaatcctgcacgacccaccaatcgctaaggtggaagcggtagtaaaacgtgaaggataacgttgcatgagcaacggcccgaagggcgagacgaagtcgagtcatcctgcacgacccaccactaacatagttagttgtagtatccagcgtagtatcgggtgattagctcagctgggagagcacctcccttacaaggagggggtcggcggttcgatcccgtcatcacccaccaccgggtcgttagctcagttggtagagcagttgacttttaatcaattggtcgcaggttcgaatcctgcacgacccaccagttttaacatcgaagacagatgttaagcgtgtaggataacgttgcgtcagcaacggcccgtagggcgagcgaagcgagtcatcctgcacgacccaccactaatgacggtgggttcggtggaagtagtttgtagtatccagcgcagtatcgggtgattagctcagctgggagagcacctcccttacaaggagggggtcggcggttcgatcccgtcatcacccaccactcgggtcgttagctcagttggtagagcagttgacttttaatcaattggtcgcaggttcgaatcctgcacgacccaccagttttaacatcaaactcagatgttaagcgtgaaggataacgttgcgccagcaacggcccgtagggcgagcgaagcgagtcatcctgcacgacccaccaatcttaaagattggccccgagtaaaaatctttcaggtaacacccgtatgggtcgttagctcagttggtagagcagttgacttttaatcaattggtcgcaggttcgaatcctgcacgacccaccaatttaaaggtggttactggtagagaacgtgaaggataacgttgcgttagcaacggcccgaagggcgagacgaagtcgagtcatcctgcacgacccaccatcctgaatgattaaggcagcataatcccgcaaggggtcgttagctcagttggtagagcagttgacttttaatcaattggtcgcaggttcgaatcctgcacgacccaccaatgtaaaaaagcgccctaaaggcgcttttt

SEQ ID NO: 24 - DNA, 502 bp, Escherichia coli
  source 1..502: mol_type = genomic DNA
catgtctccatagaatgcgcgctacttgatgccgacttagctcagtaggtagagcaactgacttgtaatcagtaggtcaccagttcgattccggtagtcggcaccatcaagtccggtggggttcccgagcggccaaagggagcagactgtaaatctgccgtcacagacttcgaaggttcgaatccttcccccaccaccaatttcggccacgcgatggcgtagcccgagacgataagttcgcttaccggctcgaataaagagagcttctctcgatattcagtgcagaatgaaaatcaggtagccgagttccaggatgcgggcatcgtataatggctattacctcagccttccaagctgatgatgcgggttcgattcccgctgcccgctccaagatgtgctgatatagctcagttggtagagcgcacccttggtaagggtgaggtcggcagttcgaatctgcctatcagcaccacttcttttctcctccctgttttttccttct

SEQ ID NO: 25 - DNA, 764 bp, Escherichia coli
  source 1..764: mol_type = genomic DNA
acgccgataaggtatcgcgaaaaaaaagatggctacgtagctcagttggttagagcacatcactcataatgatggggtcacaggttcgaatcccgtcgtagccaccatctttttttgcgggagtggcgaaattggtagacgcaccagatttaggttctggcgccgcaaggtgtgcgagttcaagtctcgcctcccgcaccattcaccagaaagcgttgtacggatggggtatcgccaagcggtaaggcaccggtttttgataccggcattccctggttcgaatccaggtaccccagccatcttcttcgagtaagcggttcaccgcccggttattggggtatcgccaagcggtaaggcaccggtttttgataccggcattccctggttcgaatccaggtaccccagccatcgaagaaacaatctggctacgtagctcagttggttagagcacatcactcataatgatggggtcacaggttcgaatcccgtcgtagccaccaaattctgaatgtatcgaatatgttcggcaaattcaaaaccaatttgttggggtatcgccaagcggtaaggcaccggattctgattccggcattccgaggttcgaatcctcgtaccccagccaatttattcaagacgcttaccttgtaagtgcacccagttggggtatcgccaagcggtaaggcaccggattctgattccggcattccgaggttcgaatcctcgtaccccagccacattaaaaaagctcgcttcggcgagctttt

SEQ ID NO: 26 - DNA, 497 bp, Escherichia coli
  source 1..497: mol_type = genomic DNA
ccgtattatccacccccgcaacggcgctaagcgcccgtagctcagctggatagagcgctgccctccggaggcagaggtctcaggttcgaatcctgtcgggcgcgccatttagtcccggcgcttgagctgcggtggtagtaataccgcgtaacaagatttgtagtggtggctatagctcagttggtagagccctggattgtgattccagttgtcgtgggttcgaatcccattagccaccccattattagaagttgtgacaatgcgaaggtggcggaattggtagacgcgctagcttcaggtgttagtgtccttacggacgtgggggttcaagtcccccccctcgcaccacgactttaaagaattgaactaaaaattcaaaaagcagtatttcggcgagtagcgcagcttggtagcgcaactggtttgggaccagtgggtcggaggttcgaatcctctctcgccgaccaattttgaaccccgcttcggcggggtttttt

SEQ ID NO: 27 - DNA, 922 bp, Escherichia coli
  source 1..922: mol_type = genomic DNA
agttcttcgaagcactcgtaagaggcgtgtggtgaggtggccgagaggctgaaggcgctcccctgctaagggagtatgcggtcaaaagctgcatccggggttcgaatccccgcctcaccgccatttgcatccgtagctcagctggatagagtactcggctacgaaccgagcggtcggaggttcgaatcctcccggatgcaccatattctacgtactttcagcgatgaaggtatggaagaggtggcggtaataaccgcaggcaccagggaggataacgttgctttagcaacggcccgaagggcgagccgcaaggcgagtaatcctcccggatgcaccatctcttacttgatacggctttagtagcggtatcaaaaaatctgcagtaaagtaagtttcccgatgcatccgtagctcagctggatagagtactcggctacgaaccgagcggtcggaggttcgaatcctcccggatgcaccatctcttacttgatatggctttagtagcggtatcaatatcagcagtaaaataaatttcccgatgcatccgtagctcagctggatagagtactcggctacgaaccgagcggtcggaggttcgaatcctcccggatgcaccatattctccgaaatcttcagcaatgaaggtatcgaagaggtagcggaattaaccgcaggcactagggatgataacgttgctttagcaacggcccgaagggcgagccgcaaggcgagtaatcctcccggatgcaccatctcttaattgatatggctttagtagcggtatcaatatcagcagtagaataagttttcccgatgcatccgtagttcagctggatagagtactcggctacgaaccgagcggtcggaggttcgaatcctcccggatgcaccatattctccgtaaccttcagcaatgaaggta

SEQ ID NO: 28 - DNA, 463 bp, Escherichia coli
  source 1..463: mol_type = genomic DNA
cctataatgcgactccacacagcgggcgggcgtagttcaatggtagaacgagagcttccccaagctctatacgagggttcgattcccttcgcccgctccaactactttatgtagtctccgccgtgtagcaagaaattgagaagtggagagatgccggagcggctgaacggaccgggattctaaccccggagtagggacaactctaccgggggttcaaatccccctctctccgccactttctcgccagctaaatttcttgtaaaaatgtgaagtaccgaagtgtccccttcgtctagaggcccaggacaccgccctaccgacggcggtaacaggggttcgaatcccctaggggacgccacttgtggctatagctcagttggtagagccctggatttcctattccagttgtcgtgggttcgaatcccattagccaccccaatgtaaaaaagcgccctaaaggcgc

SEQ ID NO: 29 - DNA, 652 bp, Escherichia coli
  source 1..652: mol_type = genomic DNA
tagtttgtagtatccagcgcagtatcgcgggcgtagttcaatggtagaacgagagcttccccaagctctatacgagggttcgattcccttcgcccgctccactcggagagatgccggagcggctgaacggaccgggattctaaccccggagtagggacaactctaccgggggttcaaatccccctctctccgccagttttaacatcaaactcagatgttaagcgtgaaggataacgttgcgccagcaacggcccgtagggcgagcgaagcgagtcatcctgcacgacccaccaatcttaaagattggccccgagtaaaaatctttcaggtaacacccgtatgtccccttcgtctagaggcccaggacaccgccctaccgacggcggtaacaggggttcgaatcccctaggggacgccaatttaaaggtggttactggtagagaacgtgaaggataacgttgcgttagcaacggcccgaagggcgagacgaagtcgagtcatcctgcacgacccaccatcctgaatgattaaggcagcataatcccgcaaggtggctatagctcagttggtagagccctggatttcctattccagttgtcgtgggttcgaatcccattagccaccccaatgtaaaaaagcgccctaaaggcgc

SEQ ID NO: 30 - DNA, 501 bp, Escherichia coli
  source 1..501: mol_type = genomic DNA
tctccatagaatgcgcgctacttgatgcgggcgtagttcaatggtagaacgagagcttccccaagctctatacgagggttcgattcccttcgcccgctccatcaagtccggagagatgccggagcggctgaacggaccgggattctaaccccggagtagggacaactctaccgggggttcaaatccccctctctccgccaatttcggccacgcgatggcgtagcccgagacgataagttcgcttaccggctcgaataaagagagcttctctcgatattcagtgcagaatgaaaatcaggtagccgagttccaggatgtccccttcgtctagaggcccaggacaccgccctaccgacggcggtaacaggggttcgaatcccctaggggacgccaagatgtgtggctatagctcagttggtagagccctggatttcctattccagttgtcgtgggttcgaatcccattagccaccccacttcttttctcctccctgttttttc

SEQ ID NO: 31 - DNA, 436 bp, Escherichia coli
  source 1..436: mol_type = genomic DNA
ttatccacccccgcaacggcgctaagcgggcgtagttcaatggtagaacgagagcttccccaagctctatacgagggttcgattcccttcgcccgctccatctttttttggagagatgccggagcggctgaacggaccgggattctaaccccggagtagggacaactctaccgggggttcaaatccccctctctccgccattcaccagaaagcgttgtacggagtccccttcgtctagaggcccaggacaccgccctaccgacggcggtaacaggggttcgaatcccctaggggacgccatcttcttcgagtaagcggttcaccgcccggttatgtggctatagctcagttggtagagccctggatttcctattccagttgtcgtgggttcgaatcccattagccaccccatcgaagaaacaatctggctacgtag

SEQ ID NO: 32 - DNA, 490 bp, Escherichia coli
  source 1..490: mol_type = genomic DNA
ttatccacccccgcaacggcgctaagcgggcgtagttcaatggtagaacgagagcttccccaagctctatacgagggttcgattcccttcgcccgctccatttagtcccggcgcttgagctgcggtggtagtaataccgcgtaacaagatttgtagtgggagagatgccggagcggctgaacggaccgggattctaaccccggagtagggacaactctaccgggggttcaaatccccctctctccgccattattagaagttgtgacaatgtccccttcgtctagaggcccaggacaccgccctaccgacggcggtaacaggggttcgaatcccctaggggacgccacgactttaaagaattgaactaaaaattcaaaaagcagtatttgtggctatagctcagttggtagagccctggatttcctattccagttgtcgtgggttcgaatcccattagccaccccaattttgaaccccgcttcggcggggt

SEQ ID NO: 33 - DNA, 636 bp, Escherichia coli
  source 1..636: mol_type = genomic DNA
ttcgaagcactcgtaagaggcgtgtgcgggcgtagttcaatggtagaacgagagcttccccaagctctatacgagggttcgattcccttcgcccgctccatttggagagatgccggagcggctgaacggaccgggattctaaccccggagtagggacaactctaccgggggttcaaatccccctctctccgccatattctacgtactttcagcgatgaaggtatggaagaggtggcggtaataaccgcaggcaccagggaggataacgttgctttagcaacggcccgaagggcgagccgcaaggcgagtaatcctcccggatgcaccatctcttacttgatacggctttagtagcggtatcaaaaaatctgcagtaaagtaagtttcccgatgtccccttcgtctagaggcccaggacaccgccctaccgacggcggtaacaggggttcgaatcccctaggggacgccatctcttacttgatatggctttagtagcggtatcaatatcagcagtaaaataaatttcccgatgtggctatagctcagttggtagagccctggatttcctattccagttgtcgtgggttcgaatcccattagccaccccatattctccgaaatcttcagcaatgaagg

SEQ ID NO: 34 - protein, 16 aa, Escherichia coli
LEYNFNSHNVGITADK

SEQ ID NO: 35 - protein, 18 aa, Escherichia coli
LEYNFNSHNVGITADKQK

SEQ ID NO: 36 - protein, 16 aa, Escherichia coli
LEYNFNSHNVGITADK

SEQ ID NO: 37 - protein, 16 aa, Escherichia coli
LEYNFNSHNVHITADK

SEQ ID NO: 38 - protein, 18 aa, Escherichia coli
LEYNFNSHNVHITADKQK

SEQ ID NO: 39 - protein, 16 aa, Escherichia coli
LEYNFNSHNVTITADK

SEQ ID NO: 40 - protein, 18 aa, Escherichia coli
LEYNFNSHNVHITADKQK

SEQ ID NO: 41 - protein, 11 aa, Escherichia coli
LEYNFNSHNVR

SEQ ID NO: 42 - protein, 16 aa, Escherichia coli
LEYNFNSHNVEITADK

SEQ ID NO: 43 - protein, 18 aa, Escherichia coli
LEYNFNSHNVEITADKQK

SEQ ID NO: 44 - protein, 11 aa, Escherichia coli
LEYNFNSHNVR

SEQ ID NO: 45 - protein, 16 aa, Escherichia coli
LEYNFNSHNVYITADK

SEQ ID NO: 46 - protein, 18 aa, Escherichia coli
LEYNFNSHNVYITADKQK

SEQ ID NO: 47 - protein, 22 aa, Escherichia coli
NFNSHNVRITADKQKNGIKANF

SEQ ID NO: 48 - protein, 22 aa, Escherichia coli
NFNSHNVRITADKQKNGIKANF

SEQ ID NO: 49 - protein, 12 aa, Escherichia coli
KLEYNFNSHNVR

SEQ ID NO: 50 - protein, 11 aa, Escherichia coli
LEYNFNSHNVW

SEQ ID NO: 51 - protein, 16 aa, Escherichia coli
LEYNFNSHNVQITADK

SEQ ID NO: 52 - protein, 18 aa, Escherichia coli
LEYNFNSHNVQITADKQK

SEQ ID NO: 53 - protein, 16 aa, Escherichia coli
LEYNFNSHNVQITADK

SEQ ID NO: 54 - protein, 18 aa, Escherichia coli
LEYNFNSHNVQITADKQK

SEQ ID NO: 55 - protein, 16 aa, Escherichia coli
LEYNFNSHNVQITADK

SEQ ID NO: 56 - protein, 18 aa, Escherichia coli
LEYNFNSHNVQITADKQK

SEQ ID NO: 57 - protein, 16 aa, Escherichia coli
LEYNFNSHNVSITADK

SEQ ID NO: 58 - protein, 18 aa, Escherichia coli
LEYNFNSHNVSITADKQK

SEQ ID NO: 59 - protein, 16 aa, Escherichia coli
LEYNFNSHNVDITADK

SEQ ID NO: 60 - protein, 16 aa, Escherichia coli
LEYNFNSHNVSITADK

SEQ ID NO: 61 - protein, 18 aa, Escherichia coli
LEYNFNSHNVSITADKQK

SEQ ID NO: 62 - protein, 16 aa, Escherichia coli
LEYNFNSHNVDITADK

SEQ ID NO: 63 - protein, 18 aa, Escherichia coli
LEYNFNSHNVSITADKQK

SEQ ID NO: 64 - protein, 16 aa, Escherichia coli
LEYNFNSHNVSITADK

SEQ ID NO: 65 - protein, 16 aa, Escherichia coli
LEYNFNSHNVSITADK

SEQ ID NO: 66 - protein, 16 aa, Escherichia coli
LEYNFNSHNVWITADK

SEQ ID NO: 67 - protein, 18 aa, Escherichia coli
LEYNFNSHNVYITADKQK

SEQ ID NO: 68 - protein, 18 aa, Escherichia coli
LEYNFNSHNVQITADKQK

SEQ ID NO: 69 - protein, 16 aa, Escherichia coli
LEYNFNSHNVQITADK

SEQ ID NO: 70 - protein, 18 aa, Escherichia coli
LEYNFNSHNVQITADKQK

SEQ ID NO: 71 - protein, 18 aa, Escherichia coli
LEYNFNSHNVYITADKQK

SEQ ID NO: 72 - protein, 16 aa, Escherichia coli
LEYNFNSHNVYITADK

SEQ ID NO: 73 - protein, 18 aa, Escherichia coli
LEYNFNSHNVYITADKQK

SEQ ID NO: 74 - protein, 18 aa, Escherichia coli
LEYNFNSHNVYITADKQK

SEQ ID NO: 75 - protein, 26 aa, Escherichia coli
TSHLNSSSSSHASVSKGEEDNMAIIK

SEQ ID NO: 76 - protein, 30 aa, Escherichia coli
TSHLNSSSSSHASVSKGEEDNMAIIKEFMR

SEQ ID NO: 77 - protein, 27 aa, Escherichia coli
TSHLNYYYYYYHASVSKGEEDNMAIIK

SEQ ID NO: 78 - protein, 26 aa, Escherichia coli
TSHLNQQQQQHASVSKGEEDNMAIIK

SEQ ID NO: 79 - protein, 30 aa, Escherichia coli
TSHLNQQQQQHASVSKGEEDNMAIIKEFMR

SEQ ID NO: 80 - protein, 18 aa, Escherichia coli
LEYNFNSHNVYITADKQK

SEQ ID NO: 81 - protein, 16 aa, Escherichia coli
LEYNFNSHNVYITADK

SEQ ID NO: 82 - protein, 13 aa, Escherichia coli
HNVEDGSVQLADH

SEQ ID NO: 83 - protein, 43 aa, Escherichia coli
IRHNVEDGSVQLADHYQQNTPIGDGPVLLPDNHYLSTQSVLSK

SEQ ID NO: 84 - protein, 30 aa, Escherichia coli
TPIGDGPVLLPDNHYLSTQSVLSKDPNEKR

SEQ ID NO: 85 - protein, 24 aa, Escherichia coli
TPIGDGPVLLPDNHYLSTQSVLSK

SEQ ID NO: 86 - protein, 29 aa, Escherichia coli
DPNEKRDHMVLLEFVTAAGITHGMDELYK

SEQ ID NO: 87 - protein, 46 aa, Escherichia coli
HNVEDGSVQLADHYQQNTPIGDGPVLLPDNHYLSTQSVLSKDPNEK

SEQ ID NO: 88 - protein, 18 aa, Escherichia coli
LEYNFNSHNVYITADKQK

SEQ ID NO: 89 - protein, 43 aa, Escherichia coli; identical to SEQ ID NO: 83

SEQ ID NO: 90 - protein, 16 aa, Escherichia coli
LEYNFNSHNVYITADK

SEQ ID NO: 91 - protein, 41 aa, Escherichia coli
HNVEDGSVQLADHYQQNTPIGDGPVLLPDNHYLSTQSVLSK

SEQ ID NO: 92 - protein, 29 aa, Escherichia coli
DPNEKRDHMVLLEFVTAAGITHGMDELYK

SEQ ID NO: 93 - protein, 8 aa, Escherichia coli
LEYNFNSR

SEQ ID NO: 94 - protein, 16 aa, Escherichia coli
LEYNFNSHNVYITADK

SEQ ID NO: 95 - protein, 41 aa, Escherichia coli; identical to SEQ ID NO: 91

SEQ ID NO: 96 - protein, 29 aa, Escherichia coli
DPNEKRDHMVLLEFVTAAGITHGMDELYK

SEQ ID NO: 97 - protein, 43 aa, Escherichia coli; identical to SEQ ID NO: 83

SEQ ID NO: 98 - protein, 29 aa, Escherichia coli
DPNEKRDHMVLLEFVTAAGITHGMDELYK

SEQ ID NO: 99 - protein, 18 aa, Escherichia coli
LEYNFNSHNVYITADKQK

SEQ ID NO: 100 - protein, 16 aa, Escherichia coli
LEYNFNSHNVYITADK

SEQ ID NO: 101 - protein, 47 aa, Escherichia coli
HNVEDGSVQLADHYQQNTPIGDGPVLLPDNHYLSTQSVLSKDPNEKR

SEQ ID NO: 102 - RNA, 77 nt, Escherichia coli
  source 1..77: mol_type = tRNA
aggggcgtagttcaattggtagagcaccggtcttctaaaaccgggtgttgggagttcgagtctctccgcccctgcca

SEQ ID NO: 103 - RNA, 77 nt, Escherichia coli
  source 1..77: mol_type = tRNA
aggggcgtagttcaattggtagaacaccggtcttctaataccgggtgttgggagttcgagtctctccgcccccgcca

SEQ ID NO: 104 - RNA, 78 nt, Escherichia coli
  source 1..78: mol_type = tRNA
gcatccgtagctcagctggatagagtactcggcttctaaaccgagcggtcggaggttcgaatcctcccggatgcacca

SEQ ID NO: 105 - RNA, 78 nt, Escherichia coli
  source 1..78: mol_type = tRNA
gcatccgtagctcagctggatagagtactcggcttctaaaccgatcggtcggaggttcgaatcctcccggatgcacca

SEQ ID NO: 106 - RNA, 78 nt, Escherichia coli
  source 1..78: mol_type = tRNA
gcatccgtagttcagctggatagagcactcggcttctaaaccgatcggtcggaggttcgaatcctcccggatgcacca

SEQ ID NO: 107 - RNA, 76 nt, Escherichia coli
  source 1..76: mol_type = tRNA
tggggtatcgccaagcggtaaggcaccggatttctaattccggcattccgaggttcgaatcctcgtaccccagcca

SEQ ID NO: 108 - RNA, 76 nt, Escherichia coli
  source 1..76: mol_type = tRNA
tggggtatcgccaagcggtaaggcaccggacttctaattccggcattccgaggttcgaatcctcgtaccccagcca

SEQ ID NO: 109 - RNA, 75 nt, Escherichia coli
  source 1..75: mol_type = tRNA
tggggtatcgccaagcggtaaggcaccggacttctaattccggcatccgaggttcgaatcctcgtaccccagcca

SEQ ID NO: 110 - RNA, 91 nt, Escherichia coli
  source 1..91: mol_type = tRNA
ggagagatgccggagcggctgaacggaccggtcttctaaaaccggagtaggggcaactctaccgggggttcaaatccccctctctccgcca

SEQ ID NO: 111 - RNA, 91 nt, Escherichia coli
  source 1..91: mol_type = tRNA
ggagagatgccggagcggctgaacggaccggtattctaacaccggagtaggggcaactctaccgggggttcaaatccccctctctccgcca

SEQ ID NO: 112 - RNA, 91 nt, Escherichia coli
  source 1..91: mol_type = tRNA
ggagagatgccggagcggctgaacggaccgctattctaacacccgactaggggtaactctaccgggggttcaaatccccctctctccgcca

SEQ ID NO: 113 - RNA, 91 nt, Escherichia coli
  source 1..91: mol_type = tRNA
ggagagatgccggagcggctgaacggaccgggattctaaccccggagtagggacaactctaccgggggttcaaatccccctctctccgcca

SEQ ID NO: 114 - RNA, 77 nt, Escherichia coli; identical to SEQ ID NO: 102
  source 1..77: mol_type = tRNA

SEQ ID NO: 115 - RNA, 77 nt, Escherichia coli; identical to SEQ ID NO: 103
  source 1..77: mol_type = tRNA

SEQ ID NO: 116 - RNA, 86 nt, Escherichia coli
  source 1..86: mol_type = tRNA
ggtggggttcccgagcggccaaagggagcagacttctaaatctgccgtcatcgacttcgaaggttcgaatccttcccccaccacca

SEQ ID NO: 117 - RNA, 86 nt, Escherichia coli
  source 1..86: mol_type = tRNA
ggtggggttcccgagcggccaaagggagcagaactctaaatctgccgtcatcgacttcgaaggttcgaatccttcccccaccacca

(19) United States
(12) Patent Application Publication
(10) Pub. No.: US 2024/0384267 A1
(43) Pub. Date: Nov. 21, 2024

(54) COMPOSITIONS AND METHODS FOR MULTIPLEX DECODING OF QUADRUPLET CODONS

(71) Applicant: The Broad Institute, Inc., Cambridge, MA (US)

(72) Inventors: Ahmed H. Badran, Cambridge, MA (US); Gavriela D. Carver, Cambridge, MA (US)

(21) Appl. No.: 18/693,195

(22) PCT Filed: Sep. 20, 2022
(86) PCT No.: PCT/US2022/076687

§ 371 Date: Mar. 19, 2024

Related U.S. Application Data
(60) Provisional application No. 63/246,005, filed on Sep. 20, 2021.

(51) Int. Cl. C12N 15/11; C12N 15/70

(52) U.S. Cl. CPC C12N 15/11; C12N 15/70

(57) ABSTRACT

The subject matter disclosed herein is generally directed to compositions and methods for multiplex decoding of quadruplet codons and methods for increasing the efficiency of quadruplet codon decoding using qtRNA evolution. Continuous evolution of qtRNAs is disclosed. Multiplex qtRNA constructs are disclosed.

### CROSS-REFERENCE TO RELATED APPLICATIONS

This application claims the benefit of U.S. Provisional Application No. 63/246,005, filed Sep. 20, 2021. The entire contents of the above-identified application are hereby fully incorporated herein by reference.

### STATEMENT REGARDING FEDERALLY SPONSORED RESEARCH

This invention was made with government support under Grant No. (s) OD024590 awarded by the National Institutes of Health and NNH17ZDA001N-EXO awarded by the NASA Exobiology Program. The government has certain rights in the invention.

### REFERENCE TO AN ELECTRONIC SEQUENCE LISTING

The contents of the electronic sequence listing (“BROD-5430WP.xml”; Size is 113,719 bytes and it was created on Sep. 13, 2022) is herein incorporated by reference in its entirety.

### TECHNICAL FIELD

The subject matter disclosed herein is generally directed to compositions and methods for multiplex decoding of quadruplet codons and methods for increasing the efficiency of quadruplet codon decoding using qtRNA evolution.

### BACKGROUND

Genetic code expansion (GCE) has enabled the programmed incorporation of non-canonical amino acids (ncAAs) in proteins in living cells. This has been achieved—in nature and in the laboratory—by recoding existing redundant stop or sense codons¹⁻⁶, increasing codon size⁷⁻¹², or by adding additional synthetic letters to the genetic language^{13, 14}. These codons enable the supplementation of the proteogenic amino acid repertoire with a variety of non-proteogenic monomeric units, including noncanonical α-amino acids^{15, 16}, β-amino acids¹⁷, and benzoic and malonyl acids¹⁸. Although >150 ncAAs have been incorporated into proteins in vivo and in vitro¹⁹, cellular incorporation remains limited to 3 unique ncAAs owing to difficulties in designating >3 readily assignable codons in a single gene²⁰.

Quadruplet codons, which may enable an expanded genetic code with up to 255 unique assignable amino acids (4⁴ permutations=255 quadruplet codons+1 stop codon), may address many of the limitations of current GCE methodologies. Briefly, tRNAs containing a point insertion in the anticodon loop can often induce a +1 frameshift during translation^{21, 22}. This frameshifting activity is maintained by correct Watson-Crick base pairing between bases in the tRNA anticodon and the mRNA codon^{10, 11, 23}, enabling the faithful decoding of a four-base codon^{8, 12, 24-27}. Recent insight into one +1 frameshifting mechanism of the SufB2 tRNAs suggests that the first three bases of a quadruplet codon are decoded followed by a frameshift during ribosome translocation on the mRNA²², although other mechanisms may exist for alternative tRNAs. Whereas this process can also depend on the sequence context within the mRNA¹², these observations suggest that it may be possible to engineer cellular protein synthesis, specifically engineering of ribosomal conformational dynamics²², toward sequence-specific quadruplet codon translation.

However, such broadly implemented systems have not been realized due to the unpredictable and often limited efficiency of quadruplet codon translation. Broadly, tRNA engineering may affect overall decoding efficiency through altered interactions with aminoacyl-tRNA synthetases^{10, 28} and elongation factors²⁹⁻³¹, perturbed ribosomal accommodation^{11, 32}, or limited mRNA decoding efficiency^{11, 32-34}. Compared to a triplet codon, translating a single quadruplet codon is substantially less efficient in unmodified, wild-type prokaryotic or eukaryotic cells; relative translational efficiencies (η, see Methods) of <3% are common. As translation efficiency for a protein of length N should scale with η^{N}, this makes the development of an exclusively four-base codon translation system challenging. Implementing such a system will therefore require efficient, well-characterized tRNAs capable of translating quadruplet codons into the corresponding canonical amino acids (cAAs), alongside diverse ncAAs for researcher-dictated functions.

Whereas many elements of the translational apparatus have been explored for improved ncAA incorporation at quadruplet codons in biological systems, few reports have investigated quadruplet-decoding tRNAs (qtRNAs) that faithfully incorporate canonical amino acids under multiplexing conditions.

Citation or identification of any document in this application is not an admission that such a document is available as prior art to the present invention.

### SUMMARY

In one aspect, the present invention provides for an engineered multicistronic expression construct comprising at least 3-4 quadruplet-decoding tRNA (qtRNA) encoding regions. In certain embodiments, the expression construct is derived from an endogenous multicistronic tRNA operon. In certain embodiments, the endogenous multicistronic tRNA is from a bacterium. In certain embodiments, the bacteria is E. coli. In certain embodiments, at least one of the qtRNAs are non-canonical amino acid (ncAA) qtRNAs. In certain embodiments, the engineered expression further comprises an RNA polymerase III promoter.

In another aspect, the present invention provides for a vector comprising the engineered expression construct of any embodiment herein. In certain embodiments, the vector is a prokaryotic expression vector. In certain embodiments, the vector is a eukaryotic expression vector. In certain embodiments, the eukaryotic expression vector is a mammalian, insect, or yeast expression vector.

In another aspect, the present invention provides a cell comprising the engineered expression construct of any of any embodiment herein, or the vector of any embodiment herein. In certain embodiments, the scaffold is integrated into the genome of the cell or capable of autonomous replication in the cell.

In another aspect, the present invention provides a method of decoding multiple orthogonal quadruplet codons in a cell comprising expressing in the cell the engineered expression construct of any of any embodiment herein, or the vector of any embodiment herein.

In another aspect, the present invention provides a method of increasing the efficiency of a quadruplet-decoding tRNA (qtRNA) comprising performing phage-assisted continuous evolution (PACE) on a qtRNA, wherein the PACE system comprises: i) an M13 selection phage (SP) encoding the qtRNA in place of gIII, ii) an accessory plasmid (AP) encoding gIII comprising one or more quadruplet codons that are required to be decoded by the qtRNA in order to produce functional pIII, and iii) an inducible mutagenesis plasmid (MP) encoding one or more mutagenic proteins capable of increasing the rate of evolution. In certain embodiments, the AP comprises 1 to 4 quadruplet codons. In certain embodiments, gIII comprises one or more quadruplet codons at a permissive residue. In certain embodiments, gIII comprises one or more quadruplet codons at a non-permissive residue. In certain embodiments, MP is the MP6 plasmid. In certain embodiments, the qtRNA is a non-canonical amino acid (ncAA) qtRNA and the PACE system further comprises an aminoacyl-tRNA synthetase for the ncAA. In certain embodiments, the starting qtRNA is selected based on decoding of a quadruplet codon in a reporter gene and inserting an amino acid required for translating a functional reporter gene. In certain embodiments, the starting qtRNA comprises a quadruplet anticodon and an amino acid specific tRNA scaffold. In certain embodiments, the method further comprises determining decoding activity of evolved qtRNAs by expressing an evolved qtRNA in a cellular system comprising a nucleotide sequence encoding a selection marker comprising one or more quadruplet codons, wherein the selection marker is functional only if the one or more quadruplet codons are decoded by the qtRNA; and comparing expression of the selection marker with a cellular system comprising a nucleotide sequence encoding the selection marker having a wild-type codon sequence. In certain embodiments. the selection marker comprises 1 to 6 quadruplet codons. In certain embodiments, the selection marker is a luminescent, fluorescent, or enzymatic selection marker. In certain embodiments, the selection marker comprises a luciferase (LuxAB), sfGFP, or β-galactosidase (LacZ) selection marker. In certain embodiments, the selection marker is a dual fluorescent selection marker comprising adjacent quadruplet codons in a linker sequence between the dual fluorescent markers. In certain embodiments, the selection marker comprises one or more quadruplet codons at a permissive residue. In certain embodiments, the selection marker comprises one or more quadruplet codons at a non-permissive residue. In certain embodiments, the cellular system is a prokaryotic or eukaryotic cellular system. In certain embodiments, the eukaryotic cellular system consists of yeast cells. In certain embodiments, the eukaryotic cellular system consists of insect cells. In certain embodiments, the eukaryotic cellular system consists of mammalian cells. In certain embodiments, the cellular system is modified to decrease or abolish expression of a cellular release factor.

In another aspect, the present invention provides a qtRNA selected from Table 4.

These and other aspects, objects, features, and advantages of the example embodiments will become apparent to those having ordinary skill in the art upon consideration of the following detailed description of example embodiments.

### BRIEF DESCRIPTION OF THE DRAWINGS

An understanding of the features and advantages of the present invention will be obtained by reference to the following detailed description that sets forth illustrative embodiments, in which the principles of the invention may be utilized, and the accompanying drawings of which:

FIG. 1A-1F—Discovery and quantification of selection derived qtRNAs. FIG. 1a) Schematic representation of cellular reporter assays. Suppressed positions in the reporter proteins are shown in parentheses. In all cases, reporter protein translation prematurely terminates in the absence of a functional qtRNA due to a stop codon generated downstream of the quadruplet codon. Conversely, a functional qtRNA affects quadruplet codon suppression, yielding a full-length protein. FIG. 1b) Validation of the luciferase reporter for quadruplet codon suppression. The previously described qtRNA^{Thr}_{ACCA}²⁵ selectively decodes the cognate quadruplet codon, whereas a codon/anticodon mismatch or native triplet tRNA yields no luminescence using the quadruplet codon at residue S357 in LuxAB. FIG. 1c) Decoding efficiency of previously described qtRNAs, qtRNA^{Gln}_{CAAA}²⁷ and qtRNA^{Gly}_{GGGG}⁸¹. FIG. 1d) The LacZ selection pipeline yielded novel, functional qtRNAs for aspartate, glutamate, histidine, and tyrosine, which were assayed using the luciferase reporter. FIG. 1e) Several LacZ selection-derived qtRNAs showed robust suppression of the cognate quadruplet codon at the permissive residue Y151 in sfGFP. FIG. 1f) The sfGFP reporter was used to quantify amino acid incorporation at position Y151 via mass spectrometry. Comprehensive peptide fragmentation spectra are reported in FIG. 8. In all cases, reporter data is normalized to an otherwise wild-type reporter protein and color coded by the used reporter. Data represent the mean and standard deviation of 3-12 biological replicates.

FIG. 2A-2G—Continuous directed evolution of qtRNAs improves quadruplet codon decoding. FIG. 2a) Engineered UAGA-qtRNAs using representative scaffolds for each of the 20 elongator tRNAs and initiator (fMet) tRNA validated using the LuxAB reporter assay with UAGA incorporated at residue S357; qtRNA^{fMet}_{UAGA} uses a reporter with UAGA at residue 1 of LuxAB. FIG. 2b) Schematic representation of qtRNA-PACE circuit. Selection phages (SPs) encoding a functional qtRNA enables the translation of a quadruplet codon in gIII (encoded on an accessory plasmid; AP), resulting in the production of full-length pIII and infectious progeny phage. FIG. 2c) SP-borne qtRNAs enable propagation as visualized by plaque assay. Mismatched qtRNA/AP pairs fail to generate phage and do not result in plaque formation. SP(−) indicates an SP lacking a qtRNA, and AP(−) indicates cells without an AP. FIG. 2d) UAGA qtRNAs with detectable activities using the LuxAB reporter were tested in SP propagation assays, showing that η≥0.5% are necessary to support phage enrichment above input (visualized as a dotted line). FIG. 2e) Quantification of SP titers and flow rates during qtRNA-PACE campaigns using AP_{1×UAGA}. Experiments were initiated with either a clonal (grey) or degenerate (black) qtRNA population, or the evolved variant Ser_{UAGA}-Evo1 (purple). The second leg of qtRNA-PACE with qtRNA^{Ser}_{UAGA} uses the more stringent AP_{3×UAGA}. MP6 was used in all qtRNA-PACE experiments. qtRNA activities prior to (starting) and following (Evo) qtRNA-PACE campaigns and their associated fold improvements as quantified using LuxAB (FIG. 2f) or sfGFP (FIG. 2g) reporters. Comprehensive sfGFP peptide fragmentation spectra for all engineered and evolved UAGA-decoding qtRNAs are reported in FIG. 12. In all cases, reporter data is normalized to an otherwise wild-type protein and color coded by the used reporter. Data represent the mean and standard deviation of 3-8 biological replicates.

FIG. 3A-3E—In vitro analysis of evolved qtRNAs. In vitro aminoacylation of qtRNAs using EcSerRS (FIG. 3a), EcArgRS (FIG. 3b), and EcTyrRS (FIG. 3c); qtRNA^{Trp}_{UAGA}was used as a negative control in all cases. FIG. 3d) Analysis of EcGlnRS identity elements as found in the engineered qtRNA^{Trp}_{UAGA} and qtRNA-PACE evolved qtRNA^{Trp}_{UAGA}-Evo1. FIG. 3e) Anticodon and adjacent nucleobase identities for known E. coli glutamine tRNAs and the qtRNA-PACE evolved qtRNA^{Trp}_{UAGA}-Evo1. Position of the modified nucleotide cmnm⁵s²U (yellow) in the co-crystal structure of tRNA^{Gln}_{CAA} and E. coli GlnRS (PDB 4JYZ). Data represent the mean and standard deviation of 3 biological replicates.

FIG. 4A-4D—Quantification of evolved qtRNA crosstalk and processivity. FIG. 4a) Evolved UAGA-qtRNAs show low crosstalk with codons bearing a different nucleotide at the fourth position but not the third position of the quadruplet codon. Evolved UAGA-qtRNAs can additionally crosstalk with amber (UAG) codons, but suppression is dependent on the base following the stop codon. FIG. 4b) Quadruplet-codon translation using a previously described orthogonal ribosome RiboQ1 (contains mutations U531G, U534A, A1196G, A1197G in the 16S rRNA)¹¹. FIG. 4c) Schematic representation of the dual sfGFP-mCherry reporter assay to investigate processivity of quadruplet codon translation. In all cases, sfGFP and mCherry are separated by a linker composed of adjacent UAGA quadruplet codons. The ratio of mCherry to sfGFP is a proxy for UAGA codon suppression efficiency and processivity. FIG. 4d) Evolved UAGA qtRNAs effectively translate linkers containing up to 5-6 adjacent UAGA quadruplet codons in the RF1+ strain S3489 (top) or the ΔRF1 strain JX33 (bottom). Comprehensive peptide fragmentation spectra are reported in FIG. 15. In all cases, reporter data is normalized to an otherwise wild-type protein and color coded by the used reporter. Data represents the mean and standard deviation of 2-8 biological replicates.

FIG. 5A-5K—Multiplex quadruplet codon suppression in sfGFP. FIG. 5a) Engineered and evolved qtRNAs showcase robust mutual orthogonality position Y151 of sfGFP. Expected codon-qtRNA interactions are outlined. FIG. 5b) Schematic representation of orthogonal qtRNA expression plasmid architecture, including qtRNA, plasmid origin, and antibiotic resistance markers. FIG. 5c) The sfGFP reporter previously described was first used to quantify amino acid incorporation at positions throughout sfGFP via mass spectrometry. Comprehensive peptide fragmentation spectra are reported in FIG. 16. Using a reporter with unique quadruplet codons in sfGFP alongside orthogonal qtRNA expression plasmids encoding qtRNA^{Gly}_{GGGG}, qtRNA^{Ser}_{UAGA-Evo}3, qtRNA^{Glu}_{CGGU}, and/or qtRNA^{His}_{AGGA} enables the translation of one (FIG. 5d), two (FIG. 5e), and three (FIG. 5f) unique quadruplet codons in a single reporter gene. FIG. 5g) Schematic representation of the engineered multicistronic qtRNA scaffold and reporter plasmid encoding multiple quadruplet codons in sfGFP. Production of full-length sfGFP is dependent on the translation of up to four quadruplet codons Expression of qtRNA^{Gly}_{GGGG}, qtRNA^{Ser}_{UAGA-Evo}3, qtRNA^{Glu}_{CGGU}, and qtRNA^{His}_{AGGA} from a single multicistronic qtRNA scaffold enables the translation of one (FIG. 5h), two (FIG. 5i), three (FIG. 5j) and four (FIG. 5k) quadruplet codons in sfGFP. Comprehensive peptide fragmentation spectra are reported in FIG. 19-22. In all cases, reporter data is normalized to an otherwise wild-type protein and color coded by the used reporter. Dotted lines on each plot represent strain autofluorescence. Data represents the mean and standard deviation of 5-20 biological replicates.

FIG. 6A-6B-Validation of LuxAB reporter and engineered qtRNAs. FIG. 6a) Constitutive LuxAB reporters bearing all twenty canonical amino acids show limited preference at positive S357, with the exception of arginine which shows a five-fold reduction in luminescence activity. S1 corresponds to the UCG serine codon and S2 corresponds to the ACG serine codon at position S357. FIG. 6b) Comparison of the engineered pProk-lacO promoter to the rhamnose operon-derived pRHA promoter. In all cases, reporter data is normalized to an otherwise wild-type protein. Data represents the mean and standard deviation of 3-16 biological replicates. AU: arbitrary units.

FIG. 7A-7C—Validation of lacZ library-cross-library selection and discovered hits. FIG. 7a) Degenerate (NNN) codon libraries were incorporated in lacZ at all the indicated positions and plated on minimal medium plates with either glucose (“Total”) or lactose+Bluo-Gal (“LacZ⁺”). Functional amino acid incorporation results in growth on minimal media plates supplemented with lactose as the sole carbon source. FIG. 7b) Comparison of glucose- and lactose-derived populations can be used to calculate the % LacZ (% LacZ=LacZ⁺/Total*100) and the % expected LacZ⁺ CFUs, assuming complete coverage of all 64 triplet codons. In cases where both values are comparable (underlined and bold), single clone Sanger sequencing confirmed that only the cognate amino acid was present in all blue (lactose catabolizing) colonies. FIG. 7c) Amino acid-specific positions were used as the basis of a library-cross-library selection, wherein each lacZ position was randomized to all possible quadruplet codons (NNNN) and each tRNA scaffold was concomitantly randomized at the anticodon loop (NNNN). Co-transformation of both libraries resulted in colony growth on minimal medium plates supplemented with lactose|Bluo-Gal in all cases except N461. Single clone sequencing at the codon (lacZ) and anticodon (qtRNA) showed the identical sequences in most cases. The reported sequences were discovered as anticodons (reverse complement), where red letters indicate mismatches found in the lacZ codon. CFU: colony forming unit.

FIG. 8A-8E—LC-MS/MS analysis of lacZ selection-derived hits. Mass spectra of sfGFP fragments resulting from qtRNA^{Gly}_{GGGG} (FIG. 8a), qtRNA^{His}_{AGGA} (FIG. 8b), qtRNA^{Thr}_{ACCA} (FIG. 8c), qtRNA^{Glu}_{CGGU} (FIG. 8d), and qtRNA^{Tyr}_{UAGA} (FIG. 8e) suppression of cognate quadruplet codon at Y151. Multiple peptides were observed in some cases and are shown for completeness.

FIG. 9A-9F—Benchmarking PACE-evolved qtRNA SPs using progressively stringent APs. FIG. 9a) Schematic representation of the accessory plasmid design, wherein either AP copy number was modified (L=wild-type RepA ˜4 copies/cell; H=RepA^{E93K} ˜27 copies/cell) or the number of quadruplet codons in pIII was progressively increased. In all cases, clonal SPs encoding the indicated engineered or evolved qtRNAs were challenged to form plaques in S3489 cells. For each SP, the threshold for plaque formation is visualized for serine (FIG. 9b), arginine (FIG. 9c), glutamine (FIG. 9d), tryptophan (FIG. 9e), and tyrosine (FIG. 9f).

FIG. 10A-10B—Analysis of engineered and evolved qtRNAs in bacterial RF1 knockout strains. FIG. 10a) Engineered and evolved UAGA-decoding qtRNAs assayed using an endpoint fluorescence reporter assay using two RF1 knockout strains (C321.ΔA⁵⁹ and JX33⁶⁹) with one RF1+strains (C321). In all cases, tRNAs were assayed alongside a reporter incorporating the quadruplet codon UAGA at sfGFP position Y151. FIG. 10b) Extension of the sfGFP reporter assay in JX33 and S3489 (control RF+) to all rationally engineered UAGA-decoding qtRNAs. In all cases, reporter data is normalized to an otherwise wild-type protein. Data represents the mean and standard deviation of 3-7 biological replicates.

FIG. 11A-11E—Models of engineered and evolved qtRNAs. Cloverleaf models of engineered UAGA qtRNAs and evolved variants: arginine (FIG. 11a), glutamine (FIG. 11b), serine (FIG. 11c), tryptophan (FIG. 11d), and tyrosine (FIG. 11e). In all cases, the engineered UAGA codon is highlighted in gray, and PACE-acquired mutations are highlighted in red. qtRNA^{Ser}_{UAGA}-Evo1 was used to initiate the experiment that produced qtRNA^{Ser}_{UAGA}-Evo2 and qtRNA^{Ser}_{UAGA}-Evo3.

FIG. 12A-12N—LC-MS/MS analysis of engineered and evolved qtRNAs. Mass spectra of the resultant sfGFP fragments from the suppression of UAGA quadruplet codon at sfGFP Y151 by the engineered and subsequently evolved qtRNAs: qtRNA^{Arg}_{UAGA} (FIG. 12a-c), qtRNA^{Gln}_{UAGA} (FIG. 12d-f), qtRNA^{Ser}_{UAGA} (FIG. 12g-j), qtRNA^{Trp}_{UAGA} (FIG. 12k,l), and qIRNA^{Tyr}_{UAGA} (FIG. 12m,n). Multiple peptides were observed in some cases and are shown for completeness.

FIG. 13A-13O—Analysis of qtRNA/codon specificity and crosstalk. Evolved UAGA-qtRNAs were tested using mismatched codon reporters to assess instances of decoding crosstalk. LuxAB reporters encoding quadruplet codons with modifications at the third position (FIG. 13a-e) or fourth position (FIG. 13f-j) showcase absolute requirement for guanine at the third position and preference for adenine at the fourth position. FIG. 13k-o) Evolved UAGA-qtRNAs continue to crosstalk with amber (UAG) stop codons, with a moderate preference for purines at the first position of the subsequent codon. In all cases, reporter data is normalized to an otherwise wild-type protein and color coded by the used reporter. Data represents the mean and standard deviation of 2-4 biological replicates.

FIG. 14A-14C—Translation using orthogonal ribosome. FIG. 14a) Translation of a reporter containing a UAGA codon at either residue 357 or residue 164, in comparison to translation of a luciferase containing UAGA codons at both locations. FIG. 14b) Using the H3 o-RBS/o-antiRBS pair (5′-AUAUGU/5′-AUGUUC), qtRNA^{Ser}_{UAGA}-Evo1 translates UAGA quadruplet codons at both S357 and S164 more efficiently than when using the host ribosome, especially for reporters with multiple frameshifts. FIG. 14c) Orthogonal ribosomes incorporating the described RiboQ1 mutations (U531G/U534A/A1196G/A1197G)¹¹ show comparable luminescence to the host wildtype ribosome for quadruplet codon translation. In all cases, the average wild-type (triplet) LuxAB reporter activity is shown as a dashed line. Data represent the mean and standard deviation of 2-4 biological replicates. OD optical density, AU arbitrary units.

FIG. 15A-15C—LC-MS/MS analysis of evolved qtRNA translating a linker containing adjacent UAGA quadruplet codons. Mass spectra of sfGFP-linked-mCherry fragments resulting from qtRNA^{Ser}_{UAGA}-Evo3 (FIG. 15a) and qtRNA^{Tyr}_{UAGA}-Evo1 (FIG. 15b) suppression of a linker containing six adjacent UAGA quadruplet codons, and qtRNA^{Gln}_{UAGA}-Evo2 (FIG. 15c) suppression of a linker containing five adjacent UAGA quadruplet codons. Mass spectra of the linker fragment resulting from qtRNA^{Arg}_{UAGA}-Evo1 and qtRNA^{Trp}_{UAGA}-Evo1 were unable to be identified, likely due to peptide hydrophobicity limiting chromatographic separation. Multiple peptides were observed in some cases and are shown for completeness.

FIG. 16A-16D—LC-MS/MS analysis of qtRNA translating cognate quadruplet codons at positions throughout sfGFP. Mass spectra of sfGFP fragments resulting from qtRNA^{His}_{AGGA} suppression of its cognate quadruplet codon at H148 (FIG. 16a), qtRNA^{Gly}_{GGGG} suppression of its cognate quadruplet codon at G174 (FIG. 16b), qtRNA^{Ser}_{UAGA}-Evo3 suppression of its cognate quadruplet codon at S202 (FIG. 16c), and qtRNA^{Glu}_{CGGU} suppression of its cognate quadruplet codon at E213 (FIG. 16d). Multiple peptides were observed in some cases and are shown for completeness.

FIG. 17—Influence of plasmid copy number on qtRNA decoding efficiencies. qtRNAs were tested alongside cognate quadruplet codons at positions in sfGFP to assess optimal plasmid copy number (in parentheses). In all cases, reporter data is normalized to an otherwise wild-type protein. Data represents the mean and standard deviation of 8 biological replicates.

FIG. 18—Quantification of multicistronic qtRNA scaffold-based suppression. All qtRNA scaffolds were assayed against quadruplet codons introduced at position Y151 of sfGFP. In all cases, reporter data is normalized to an otherwise wild-type protein. Data represents the mean and standard deviation of 5 biological replicates.

FIG. 19A-19D—LC-MS/MS analysis of qtRNA scaffold translating quadruplet codons at positions throughout sfGFP. Mass spectra of sfGFP fragments resulting from qtRNA scaffold #2 (composed of qtRNA^{Gly}_{GGGG}, qtRNA^{Ser}_{UAGA}-Evo3, qtRNA^{Glu}_{CGGU}, and qtRNA^{His}_{AGGA} stitched together) suppression of cognate quadruplet codons at H148, G174, and S202 (FIG. 19a), H148, G174, and E213 (FIG. 19b), H148, S202, and E213 (FIG. 19c), and G174, S202, and E213 (FIG. 19d). Multiple peptides were observed in some cases and are shown for completeness.

FIG. 20A-20D—Amino acid incorporation analysis corresponding to translation of three quadruplet codons in sfGFP. Amino acid composition analysis of qtRNA scaffold #2 (composed of qtRNA^{Gly}_{GGGG}, qtRNA^{Ser}_{UAGA}-Evo3, qtRNA^{Glu}_{CGGU}, and qtRNA^{His}_{AGGA} stitched together) suppression of cognate quadruplet codons at H148, G174, and S202 (FIG. 20a), H148, G174, and E213 (FIG. 20b), H148, S202, and E213 (FIG. 20c), and G174, S202, and E213 (FIG. 20d).

FIG. 21—LC-MS/MS analysis of qtRNA scaffold translating four quadruplet codons at positions throughout sfGFP. Mass spectra of sfGFP fragments resulting from qtRNA scaffold #2 (composed of qtRNA^{Gly}_{GGGG}, qtRNA^{Ser}_{UAGA}-Evo3, qtRNA^{Glu}_{CGGU}, and qtRNA^{His}_{AGGA} stitched together) suppression of cognate quadruplet codons at H148, G174, S202 and E213. Multiple peptides were observed in some cases and are shown for completeness.

FIG. 22A-22D—Amino acid incorporation analysis corresponding to translation of four quadruplet codons in sfGFP. Amino acid composition analysis of qtRNA scaffold #2 (composed of qtRNA^{Gly}_{GGGG}, qtRNA^{Ser}_{UAGA}-Evo3, qtRNA^{Glu}_{CGGU}, and qtRNA^{His}_{AGGA} stitched together) suppression of cognate quadruplet codons at H148 (FIG. 22a), G174 (FIG. 22b), S202 (FIG. 22c), and E213 (FIG. 22d).

The figures herein are for illustrative purposes only and are not necessarily drawn to scale.

### DETAILED DESCRIPTION OF THE EXAMPLE EMBODIMENTS

### General Definitions

Unless defined otherwise, technical and scientific terms used herein have the same meaning as commonly understood by one of ordinary skill in the art to which this disclosure pertains. Definitions of common terms and techniques in molecular biology may be found in Molecular Cloning: A Laboratory Manual, 2^{nd} edition (1989) (Sambrook, Fritsch, and Maniatis); Molecular Cloning: A Laboratory Manual, 4^{th} edition (2012) (Green and Sambrook); Current Protocols in Molecular Biology (1987) (F. M. Ausubel et al. eds.); the series Methods in Enzymology (Academic Press, Inc.): PCR 2: A Practical Approach (1995) (M. J. MacPherson, B. D. Hames, and G. R. Taylor eds.): Antibodies, A Laboratory Manual (1988) (Harlow and Lane, eds.): Antibodies A Laboratory Manual, 2^{nd} edition 2013 (E. A. Greenfield ed.); Animal Cell Culture (1987) (R. I. Freshney, ed.); Benjamin Lewin, Genes IX, published by Jones and Bartlet, 2008 (ISBN 0763752223); Kendrew et al. (eds.), The Encyclopedia of Molecular Biology, published by Blackwell Science Ltd., 1994 (ISBN 0632021829); Robert A. Meyers (ed.), Molecular Biology and Biotechnology: a Comprehensive Desk Reference, published by VCH Publishers, Inc., 1995 (ISBN 9780471185710); Singleton et al., Dictionary of Microbiology and Molecular Biology 2^{nd} ed., J. Wiley & Sons (New York, N.Y. 1994), March, Advanced Organic Chemistry Reactions, Mechanisms and Structure 4^{th} ed., John Wiley & Sons (New York, N.Y. 1992); and Marten H. Hofker and Jan van Deursen, Transgenic Mouse Methods and Protocols, 2^{nd} edition (2011).

As used herein, the singular forms “a”, “an”, and “the” include both singular and plural referents unless the context clearly dictates otherwise.

The term “optional” or “optionally” means that the subsequent described event, circumstance or substituent may or may not occur, and that the description includes instances where the event or circumstance occurs and instances where it does not.

The recitation of numerical ranges by endpoints includes all numbers and fractions subsumed within the respective ranges, as well as the recited endpoints.

The terms “about” or “approximately” as used herein when referring to a measurable value such as a parameter, an amount, a temporal duration, and the like, are meant to encompass variations of and from the specified value, such as variations of +/−10% or less, +/−5% or less, +/−1% or less, and +/−0.1% or less of and from the specified value, insofar such variations are appropriate to perform in the disclosed invention. It is to be understood that the value to which the modifier “about” or “approximately” refers is itself also specifically, and preferably, disclosed.

As used herein, a “biological sample” may contain whole cells and/or live cells and/or cell debris. The biological sample may contain (or be derived from) a “bodily fluid”. The present invention encompasses embodiments wherein the bodily fluid is selected from amniotic fluid, aqueous humour, vitreous humour, bile, blood serum, breast milk, cerebrospinal fluid, cerumen (earwax), chyle, chyme, endolymph, perilymph, exudates, feces, female ejaculate, gastric acid, gastric juice, lymph, mucus (including nasal drainage and phlegm), pericardial fluid, peritoneal fluid, pleural fluid, pus, rheum, saliva, sebum (skin oil), semen, sputum, synovial fluid, sweat, tears, urine, vaginal secretion, vomit and mixtures of one or more thereof. Biological samples include cell cultures, bodily fluids, cell cultures from bodily fluids. Bodily fluids may be obtained from a mammal organism, for example by puncture, or other collecting or sampling procedures.

The terms “subject,” “individual,” and “patient” are used interchangeably herein to refer to a vertebrate, preferably a mammal, more preferably a human. Mammals include, but are not limited to, murines, simians, humans, farm animals, sport animals, and pets. Tissues, cells and their progeny of a biological entity obtained in vivo or cultured in vitro are also encompassed.

Various embodiments are described hereinafter. It should be noted that the specific embodiments are not intended as an exhaustive description or as a limitation to the broader aspects discussed herein. One aspect described in conjunction with a particular embodiment is not necessarily limited to that embodiment and can be practiced with any other embodiment(s). Reference throughout this specification to “one embodiment”, “an embodiment,” “an example embodiment,” means that a particular feature, structure or characteristic described in connection with the embodiment is included in at least one embodiment of the present invention. Thus, appearances of the phrases “in one embodiment,” “in an embodiment,” or “an example embodiment” in various places throughout this specification are not necessarily all referring to the same embodiment, but may. Furthermore, the particular features, structures or characteristics may be combined in any suitable manner, as would be apparent to a person skilled in the art from this disclosure, in one or more embodiments. Furthermore, while some embodiments described herein include some but not other features included in other embodiments, combinations of features of different embodiments are meant to be within the scope of the invention. For example, in the appended claims, any of the claimed embodiments can be used in any combination.

All publications, published patent documents, and patent applications cited herein are hereby incorporated by reference to the same extent as though each individual publication, published patent document, or patent application was specifically and individually indicated as being incorporated by reference.

### Overview

Embodiments disclosed herein provide for multicistronic expression constructs to express more than one qtRNAs for multiplexing quadruplet codon decoding. Embodiments disclosed herein provide for methods of increasing the efficiency of quadruplet codon decoding using qtRNA evolution. Embodiments disclosed herein provide for novel qtRNAs.

Genetic code expansion technologies supplement the natural codon repertoire with assignable variants in vivo, but are often limited by heterologous translational components and low suppression efficiencies. Here, Applicants explore engineered Escherichia coli tRNAs supporting quadruplet codon translation by first developing a library-cross-library selection to nominate new quadruplet codon-anticodon pairs. Applicants extended the findings using a phage-assisted continuous evolution strategy for quadruplet-decoding tRNA evolution (qtRNA-PACE) that improved quadruplet codon translation efficiencies up to 80-fold. Evolved qtRNAs appear to maintain codon-anticodon base pairing, are typically aminoacylated by their cognate tRNA synthetases, and enable processive translation of adjacent quadruplet codons. Using these components, Applicants showcase the multiplexed decoding of up to four unique quadruplet codons by their corresponding qtRNAs in a single reporter. Cumulatively, the findings highlight how E. coli tRNAs can be engineered, evolved, and combined to decode quadruplet codons.

Applicants show for the first time that qtRNAs that are continuously evolved have greatly increased efficiency in decoding quadruplet codons, such that a quadruplet codon system can be used for incorporating any canonical or non-canonical amino acid. Furthermore, Applicants show for the first time the multiplexing of orthogonal qtRNAs using specific multicistronic constructs. The compositions and methods described herein can be used to generate proteins comprising non-canonical amino acids (e.g., recombinant proteins, and antibodies). In one example embodiment, a qtRNA can be generated that efficiently decodes a non-canonical amino acid using a quadruplet codon in vivo. Moreover, in another example embodiment, at least three or four orthogonal qtRNAs can be used to efficiently decode at least three or four non-canonical amino acids using orthogonal quadruplet codons in vivo.

### Non-Canonical Amino Acids and Aminoacyl-tRNA Synthetases

### Non-Canonical Amino Acids

Modifying proteins or polypeptides to include non-canonical amino acids (ncAAs) has great potential for use in human therapeutics, agriculture, biofuel, and other areas. As used herein, “non-canonical amino acid,” “amino acid analog,” “non-standard amino acid,” “non-natural amino acid,” “unnatural amino acid,” and the like may all be used interchangeably, and is meant to include all amino acid-like compounds that are similar in structure and/or overall shape to one or more of the twenty L-amino acids commonly found in naturally occurring proteins (Ala or A, Cys or C, Asp or D, Glu or E, Phe or F, Gly or G, H is or H, Ile or I, Lys or K, Leu or L, Met or M, Asn or N, Pro or P, Gln or Q, Arg or R, Ser or S, Thr or T, Val or V, Trp or W, Tyr or Y). Amino acid analog can also be natural amino acids with modified side chains or backbones. The methods and compositions utilizing qtRNAs described herein can be used for the efficient incorporation of one or more canonical or ncAAs for use in protein engineering. As used herein “protein engineering” refers to the modification of the structural, catalytic and/or binding properties of natural proteins and the de novo design of artificial proteins. Protein engineering relies on an efficient recognition mechanism for incorporating mutant amino acids in the desired protein sequences. Though this process has been very useful for designing new macromolecules with precise control of composition and architecture, a major limitation is that the mutagenesis is restricted to the 20 naturally occurring amino acids. However, the incorporation of non-canonical amino acids (ncAAs) can extend the scope and impact of protein engineering methods In one example, a qtRNA specific for an ncAA can be evolved according to the methods described further herein. In another example, a qtRNA specific for an ncAA can be included in a multiplex construct as described further herein.

Example ncAAs that can be incorporated in proteins have been described (see, e.g., U.S. Pat. No. 8,980,581B2). Further, single ncAAs encoded for by quadruplet codons have been incorporated into proteins in worms (see, e.g., Using a Quadruplet Codon to Expand the Genetic Code of an Animal, Zhiyan Xi, Lloyd Davis, Kieran Baxter, Ailish Tynan, Angeliki Goutou, Sebastian Greiss. bioRxiv 2021.07.17.452788).

Non-canonical amino acids amino acids carrying a wide variety of novel functional groups have been globally replaced for residue-specific replacement or incorporation into recombinant proteins. Biosynthetic assimilation of non-canonical amino acids into proteins has been achieved largely by exploiting the capacity of the wild type synthesis apparatus to utilize analogs of naturally occurring amino acids (Budisa 1995, Eur. J. Biochem 230:788-796; Deming 1997, J. Macromol. Sci. Pure Appl. Chem A34; 2143-2150; Duewel 1997, Biochemistry 36:3404-3416; van Hest and Tirrell 1998, FEBS Lett 428 (1-2): 68-70; Sharma et al., 2000, FEBS Lett 467 (1): 37-40).

A wide variety of non-canonical or non-natural amino acids can be used with evolved qtRNAs and multiplex constructs as disclosed herein. The ncAAs can be chosen based on desired characteristics of the ncAAs, e.g., function of the ncAAs, such as modifying protein biological properties such as toxicity, biodistribution, immunogenicity, or half-life, structural properties, spectroscopic properties, chemical and/or photochemical properties, catalytic properties, ability to react with other molecules (either covalently or noncovalently), or the like. Typically, the ncAAs utilized herein for certain embodiments may be selected or designed to provide additional characteristics unavailable in the twenty natural amino acids. For example, ncAAs are optionally designed or selected to modify the biological properties of a protein, e.g., into which they are incorporated. For example, the following properties are optionally modified by inclusion of an ncAA into a protein: toxicity, biodistribution, solubility, stability, e.g., thermal, hydrolytic, oxidative, resistance to enzymatic degradation, and the like, facility of purification and processing, structural properties, spectroscopic properties, chemical and/or photochemical properties, catalytic activity, redox potential, half-life, ability to react with other molecules, e.g., covalently or noncovalently, and the like. Non-canonical amino acids, once selected, can either be purchased from vendors, or chemically synthesized.

The generic structure of an alpha-amino acid contains an amino group and a carboxylic acid group that are separated by one carbon, called the α-carbon. The twenty standard amino acids differ in the makeup of the side chain (R group) attached to the α-carbon. The ncAAs disclosed herein typically differ from the natural amino acids in side chain only. Thus, the ncAAs form amide bonds with other amino acids, e.g., natural or unnatural, in the same manner in which they are formed in naturally occurring proteins. In example embodiments, the ncAAs have side chain groups that distinguish them from the natural amino acids. For example, R, optionally comprises an alkyl-, aryl-, aryl halide, vinyl halide, alkyl halide, acetyl, ketone, aziridine, nitrile, nitro, halide, acyl-, keto-, azido-, hydroxyl-, hydrazine, cyano-, halo-, hydrazide, alkenyl, alkynyl, ether, thioether, epoxide, sulfone, boronic acid, boronate ester, borane, phenylboronic acid, thiol, seleno-, sulfonyl-, borate, boronate, phospho, phosphono, phosphine, heterocyclic-, pyridyl, naphthyl, benzophenone, a constrained ring such as a cyclooctyne, thioester, enone, imine, aldehyde, ester, thioacid, hydroxylamine, amino, carboxylic acid, alpha-keto carboxylic acid, alpha or beta unsaturated acids and amides, glyoxylamide, or organosilane group, or the like or any combination thereof. Aryl substitutions may occur at various positions, e.g. ortho, meta, para, and with one or more functional groups placed on the aryl ring. Other ncAAs of interest include, but are not limited to, amino acids comprising a photoactivatable cross-linker, spin-labeled amino acids, dye-labeled amino acids, fluorescent amino acids, metal binding amino acids, metal-containing amino acids, radioactive amino acids, amino acids with novel functional groups, amino acids with altered hydrophilicity, hydrophobicity, polarity, or ability to hydrogen bond, amino acids that covalently or noncovalently interact with other molecules, photocaged and/or photoisomerizable amino acids, amino acids comprising biotin or a biotin analogue, glycosylated amino acids such as a sugar substituted serine, other carbohydrate modified amino acids, keto containing amino acids, amino acids comprising polyethylene glycol or a polyether, a polyalcohol, or a polysaccharide, amino acids that can undergo metathesis, amino acids that can undergo cycloadditions, heavy atom substituted amino acids, chemically cleavable and/or photocleavable amino acids, amino acids with an elongated side chains as compared to natural amino acids, e.g., polyethers or long chain hydrocarbons, e.g., greater than about 5 or greater than about 10 carbons, carbon-linked sugar-containing amino acids, redox-active amino acids, amino thioacid containing amino acids, amino acids containing a drug moiety, and amino acids comprising one or more toxic moieties.

In addition to unnatural amino acids that contain novel side chains, unnatural amino acids also optionally comprise modified backbone structures. Unnatural amino acids of this type include, but are not limited to, α-hydroxy acids, α-thioacids α-aminothiocarboxylates, or α-α-disubstituted amino acids, with side chains corresponding e.g. to the twenty natural amino acids or to unnatural side chains. They also include but are not limited to β-amino acids or γ-amino acids, such as substituted β-alanine and γ-amino butyric acid. In addition, substitutions or modifications at the α-carbon optionally include L or D isomers, such as D-glutamate, D-alanine, D-methyl-O-tyrosine, aminobutyric acid, and the like. Other structural alternatives include cyclic amino acids, such as proline analogs as well as 3-, 4-, 6-, 7-, 8-, and 9-membered ring proline analogs. Some non-natural amino acids, such as aryl halides (p-bromo-phenylalanine, p-iodophenylalanine, provide versatile palladium catalyzed cross-coupling reactions with ethyne or acetylene reactions that allow for formation of carbon-carbon, carbon-nitrogen and carbon-oxygen bonds between aryl halides and a wide variety of coupling partners.

In another example embodiment, many unnatural amino acids are based on natural amino acids, such as tyrosine, glutamine, phenylalanine, and the like. Tyrosine analogs include para-substituted tyrosines, ortho-substituted tyrosines, and meta substituted tyrosines, wherein the substituted tyrosine comprises an acetyl group, a benzoyl group, an amino group, a hydrazine, an hydroxyamine, a thiol group, a carboxy group, an isopropyl group, a methyl group, a C6-C20 straight chain or branched hydrocarbon, a saturated or unsaturated hydrocarbon, an O-methyl group, a polyether group, a nitro group, or the like. In addition, multiply substituted aryl rings are also contemplated. Glutamine analogs include, but are not limited to, α-hydroxy derivatives, β-substituted derivatives, cyclic derivatives, and amide substituted glutamine derivatives. Exemplary phenylalanine analogs include, but are not limited to, meta-substituted phenylalanines, wherein the substituent comprises a hydroxy group, a methoxy group, a methyl group, an allyl group, an acetyl group, or the like.

Specific examples of ncAAs include, but are not limited to, o, m and/or p forms of amino acids or amino acid analogs (non-natural amino acids), including homoallylglycine, cis- or trans-crotylglycine, 6,6,6-trifluoro-2-aminohexanoic acid, 2-aminopheptanoic acid, norvaline, norleucine, O-methyl-L-tyrosine, o-, m-, or p-methyl-phenylalanine, O-4-allyl-L-tyrosine, a 4-propyl-L-tyrosine, a tri-O-acetyl-GlcNAcβ-serine, an L-Dopa, a fluorinated phenylalanine, an isopropyl-L-phenylalanine, a p-azidophenylalanine, a p-acyl-L-phenylalanine, a p-benzoyl-L-phenylalanine, an L-phosphoserine, a phosphonoserine, a phosphonotyrosine, a p-iodo-phenylalanine, o-, m-, or p-bromophenylalanine, 2-, 3-, or 4-pyridylalanine, p-idiophenylalanine, diaminobutyric acid, aminobutyric acid, benzofuranylalanine, 3-bromo-tyrosine, 3-(6-chloroindolyl)alanine, 3-(6-bromoindolyl)alanine, 3-(5-bromonindolyl)alanine, p-chlorophenylalanine, p-ethynyl-phenylalanine, p-propargly-oxy-phenylalanine, m-ethynyl-phenylalanine, 6-ethynyl-tryptophan, 5-ethynyl-tryptophan, (R)-2-amino-3-(4-ethynyl-1H-pyrol-3-yl)propanoic acid, azidonorleucine, azidohomoalanine, p-acetylphenylalanine, p-amino-L-phenylalanine, homoproparglyglycine, p-ethyl-phenylalanine, p-ethynyl-phenylalanine, p-propargly-oxy-phenylalanine, isopropyl-L-phenylalanine, an 3-(2-naphthyl)alanine, 3-(1-naphthyl)alanine, 3-idio-tyrosine, O-propargyl-tyrosine, homoglutamine, an O-4-allyl-L-tyrosine, a 4-propyl-L-tyrosine, a 3-nitro-L-tyrosine, a tri-O-acetyl-GlcNAcβ-serine, an L-Dopa, a fluorinated phenylalanine, an isopropyl-L-phenylalanine, a p-azido-L-phenylalanine, a p-acyl-L-phenylalanine, a p-acetyl-L-phenylalanine, an m-acetyl-L-phenylalanine, selenomethionine, telluromethionine, selenocysteine, an alkyne phenylalanine, an O-allyl-L-tyrosine, an O-(2-propynyl)-L-tyrosine, a p-ethylthiocarbonyl-L-phenylalanine, a p-(3-oxobutanoyl)-L-phenylalanine, a p-benzoyl-L-phenylalanine, an L-phosphoserine, a phosphonoserine, a phosphonotyrosine, homoproparglyglycine, azidohomoalanine, a p-iodo-phenylalanine, a p-bromo-L-phenylalanine, dihydroxy-phenylalanine, dihydroxyl-L-phenylalanine, a p-nitro-L-phenylalanine, an m-methoxy-L-phenylalanine, a p-iodo-phenylalanine, a p-bromophenylalanine, a p-amino-L-phenylalanine, and an isopropyl-L-phenylalanine, trifluoroleucine, norleucine, 4-, 5-, or 6-fluoro-tryptophan, 4-aminotryptophan, 5-hydroxytryptophan, biocytin, aminooxyacetic acid, m-hydroxyphenylalanine, m-allyl phenylalanine, m-methoxyphenylalanine group, β-GlcNAc-serine, α-GalNAc-threonine, p-acetoacetylphenylalanine, para-halo-phenylalanine, seleno-methionine, ethionine, S-nitroso-homocysteine, thia-proline, 3-thienyl-alanine, homo-allyl-glycine, trifluoroisoleucine, trans and cis-2-amino-4-hexenoic acid, 2-butynyl-glycine, allyl-glycine, para-azido-phenylalanine, para-cyano-phenylalanine, para-ethynyl-phenylalanine, hexafluoroleucine, 1,2,4-triazole-3-alanine, 2-fluoro-histidine, L-methyl histidine, 3-methyl-L-histidine, β-2-thienyl-L-alanine, β-(2-thiazolyl)-DL-alanine, homoproparglyglycine (HPG) and azidohomoalanine (AHA) and the like. The structures of a variety of non-limiting ncAAs are provided in the figures, e.g., FIGS. 29, 30, and 31 of US2003/0108885A1, the entire content of which is incorporated herein by reference.

Tyrosine analogs include, but are not limited to, para-substituted tyrosines, ortho-substituted tyrosines, and meta substituted tyrosines, wherein the substituted tyrosine comprises an acetyl group, a benzoyl group, an amino group, a hydrazine, an hydroxyamine, a thiol group, a carboxy group, an isopropyl group, a methyl group, a C6-C20 straight chain or branched hydrocarbon, a saturated or unsaturated hydrocarbon, an O-methyl group, a polyether group, a nitro group, or the like. In addition, multiply substituted aryl rings are also contemplated. Glutamine analogs include, but are not limited to, α-hydroxy derivatives, β-substituted derivatives, cyclic derivatives, and amide substituted glutamine derivatives. Example phenylalanine analogs include, but are not limited to, meta-substituted phenylalanines, wherein the substituent comprises a hydroxy group, a methoxy group, a methyl group, an allyl group, an acetyl group, or the like.

Additionally, other examples optionally include (but are not limited to) an unnatural analog of a tyrosine amino acid; an unnatural analog of a glutamine amino acid; an unnatural analog of a phenylalanine amino acid; an unnatural analog of a serine amino acid; an unnatural analog of a threonine amino acid; an alkyl, aryl, acyl, azido, cyano, halo, hydrazine, hydrazide, hydroxyl, alkenyl, alkynl, ether, thiol, sulfonyl, seleno, ester, thioacid, borate, boronate, phospho, phosphono, phosphine, heterocyclic, enone, imine, aldehyde, hydroxylamine, keto, or amino substituted amino acid, or any combination thereof; an amino acid with a photoactivatable cross-linker; a spin-labeled amino acid; a fluorescent amino acid; an amino acid with a novel functional group; an amino acid that covalently or noncovalently interacts with another molecule; a metal binding amino acid; a metal-containing amino acid; a radioactive amino acid; a photocaged amino acid; a photoisomerizable amino acid; a biotin or biotin-analog containing amino acid; a glycosylated or carbohydrate modified amino acid; a keto containing amino acid; an amino acid comprising polyethylene glycol; an amino acid comprising polyether; a heavy atom substituted amino acid; a chemically cleavable or photocleavable amino acid; an amino acid with an elongated side chain; an amino acid containing a toxic group; a sugar substituted amino acid, e.g., a sugar substituted serine or the like; a carbon-linked sugar-containing amino acid; a redox-active amino acid; an α-hydroxy containing acid; an amino thio acid containing amino acid; an α,+ disubstituted amino acid; a β-amino acid; and a cyclic amino acid.

### Aminoacyl-tRNA Synthetases

Aminoacyl-tRNA synthetases (used interchangeably herein with AARS, RS or “synthetase”) catalyze the aminoacylation reaction for incorporation of amino acids into proteins via the corresponding transfer RNA molecules. Precise manipulation of synthetase activity can alter the aminoacylation specificity to stably attach ncAAs into the intended tRNA Then, through codon-anticodon interaction between message RNA (mRNA) and tRNA, the ncAAs can be delivered into a growing polypeptide chain. Thus, incorporation of ncAAs into proteins relies on the manipulation of amino acid specificity of aminoacyl tRNA synthetases. The aminoacyl-tRNA synthetase used in certain methods disclosed herein can be a naturally occurring synthetase derived from an organism, whether the same (homologous) or different (heterologous), a mutated or modified synthetase, or a designed synthetase.

Aminoacyl-tRNA synthetases must perform their tasks with high accuracy. Many of these enzymes recognize their tRNA molecules using the anticodon. These enzymes make about one mistake in 10,000. A crystal structure defines the orientation of the natural substrate amino acid in the binding pocket of a synthetase, as well as the relative position of the amino acid substrate to the synthetase residues, especially those residues in and around the binding pocket. To design the binding pocket for the ncAAs, it is preferred that these ncAAs bind to the synthetase in the same orientation as the natural substrate amino acid, since this orientation may be important for the adenylation step. The crystal structures of nearly all 20 different AARS enzymes are currently available in the Brookhaven Protein Data Bank (PDB, see Bernstein et al., J. Mol. Biol. 112: 535-542, 1977). In addition, a database of known aminoacyl tRNA synthetases has been published by Maciej Szymanski, Marzanna A. Deniziak and Jan Barciszewski, in Nucleic Acids Res. 29:288-290, 2001 (titled “Aminoacyl-tRNA synthetases database”).

In example embodiments, the synthetase used can recognize the desired ncAA selectively over related amino acids available. For example, when the ncAA to be used is structurally related to a naturally occurring amino acid, the synthetase should charge the exogenous qtRNA molecule with the desired ncAA with an efficiency at least substantially equivalent to that of, and more preferably at least about twice, 3 times, 4 times, 5 times or more than that of the naturally occurring amino acid. However, in cases in which a well-defined protein product is not necessary, the synthetase can have relaxed specificity for charging amino acids.

A synthetase can be obtained by a variety of techniques known to one of skill in the art, including combinations of such techniques as, for example, computational methods, selection methods, and incorporation of synthetases from other organisms (see, e.g., U.S. Pat. No. 8,980,581B2).

In certain embodiments, synthetases can be used or developed that efficiently charge tRNA molecules that are not charged by synthetases of the host cell. For example, suitable pairs may be generally developed through modification of synthetases from organisms distinct from the host cell. In certain embodiments, the synthetase can be developed by selection procedures. In certain embodiments, the synthetase can be designed using computational techniques such as those described in Datta et al., J. Am. Chem. Soc. 124: 5652-5653, 2002, and in U.S. Pat. No. 7,139,665, hereby incorporated by reference.

There are a variety of computational methods that can be readily adapted for identifying the structure of ncAAs that would have appropriate steric and electronic properties to interact with the substrate binding site of a modified AARS (See, e.g., Cohen et al. (1990) J. Med. Cam. 33: 883-894; Kuntz et al. (1982) J. Mol. Biol 161: 269-288; DesJarlais (1988) J. Med. Cam. 31: 722-729; Bartlett et al. (1989) (Spec. Publ., Roy. Soc. Chem.) 78: 182-196; Goodford et al. (1985) J. Med. Cam. 28: 849-857; DesJarlais et al. J. Med. Cam. 29: 2149-2153).

Another example strategy used to generate a modified tRNA/RS pair involves importing a tRNA and/or synthetase from another organism into the translation system of interest, such as Escherichia coli. In this particular example, the heterologous synthetase candidate does not charge Escherichia coli tRNA reasonably well or not at all, and the heterologous tRNA is not acylated by Escherichia coli synthetase to a reasonable extent or not at all. Schimmel et al. reported that Escherichia coli GlnRS (EcGlnRS) does not acylate Saccharomyces cerevisiae tRNAGln (See, E. F. Whelihan and P. Schimmel, EMBO J., 16:2968 (1997)). Additionally, the Saccharomyces cerevisiae amber suppressor tRNAGIn (SctRNAGlnCUA) was analyzed to determine whether it is also not a substrate for EcGlnRS. In vitro aminoacylation assays showed this to be the case; and in vitro suppression studies show that the SctRNAGlnCUA is competent in translation (see, e.g., Liu and Schultz, PNAS. USA, 96:4780 (1999)). RajBhandary and coworkers found that an amber mutant of human initiator tRNA^{fMet} is acylated by Escherichia coli GlnRS and acts as an amber suppressor in yeast cells only when EcGlnRS is coexpressed (see, Kowal, et al., PNAS USA, 98:2268 (2001)).

In an example embodiment, the starting qtRNA framework can be obtained from a different organism than the intended host cell and an AARS from the same organism can be used to charge the qtRNA with a ncAA. Further, if a qtRNA is developed with a bacterial tRNA, the qtRNA can be used in another cell type, such as yeast, but the AARS from the bacteria may need to be expressed in the yeast cells. Additionally, if a qtRNA is developed in bacterial cells with a yeast tRNA, a yeast AARS may need to be expressed in the bacterial cells. Evolution of qtRNAs is described further herein.

The practice of using orthogonal translation systems that are suitable for making proteins that comprise one or more unnatural amino acid is generally known in the art, as are the general methods for producing orthogonal translation systems. For example, see International Publication Numbers WO 2002/086075, entitled “METHODS AND COMPOSITION FOR THE PRODUCTION OF ORTHOGONAL tRNA-AMINOACYL-tRNA SYNTHETASE PAIRS;” WO 2002/085923, entitled “IN VIVO INCORPORATION OF UNNATURAL AMINO ACIDS;” WO 2004/094593, entitled “EXPANDING THE EUKARYOTIC GENETIC CODE;” WO 2005/019415, filed Jul. 7, 2004; WO 2005/007870, filed Jul. 7, 2004; WO 2005/007624, filed Jul. 7, 2004 and WO 2006/110182, filed Oct. 27, 2005, entitled “ORTHOGONAL TRANSLATION COMPONENTS FOR THE VIVO INCORPORATION OF UNNATURAL AMINO ACIDS.” Each of these applications is incorporated herein by reference in its entirety. For additional discussion of orthogonal translation systems that incorporate unnatural amino acids, and methods for their production and use, see also, Wang and Schultz, “Expanding the Genetic Code,” Chem. Commun. (Carob.) 1:1-11 (2002); Wang and Schultz “Expanding the Genetic Code,” Angewandte Chemie Int. Ed., 44(1):34-66 (2005); Xie and Schultz, “An Expanding Genetic Code,” Methods36(3):227-238 (2005); Xie and Schultz, “Adding Amino Acids to the Genetic Repertoire,” Curr. Opinion in Chemical Biology 9 (6): 548-554 (2005); Wang et al., “Expanding the Genetic Code,” Annu. Rev. Biophys. Biomol. Struct., 35:225-249 (2006); and Xie and Schultz, “A Chemical Toolkit for Proteins-an Expanded Genetic Code,” Nat. Rev. Mol. Cell. Biol., 7(10):775-782 (2006). Orthogonal AARSs that can attach a non-canonical amino acid (ncAA) to its cognate tRNA are known (see, e.g., U.S. Pat. No. 9,102,932B2; Cervettini D, Tang S, Fried S D, et al. Rapid discovery and evolution of orthogonal aminoacyl-tRNA synthetase-tRNA pairs. Nat Biotechnol. 2020; 38(8):989-999, Ding W, Zhao H, Chen Y, et al. Chimeric design of pyrrolysyl-tRNA synthetase/tRNA pairs and canonical synthetase/tRNA pairs for genetic code expansion. Nat Commun. 2020; 11(1):3154. Published 2020 Jun. 22; Melnikov S V, Söll D. Aminoacyl-tRNA Synthetases and tRNAs for an Expanded Genetic Code: What Makes them Orthogonal? Int J Mol Sci. 2019; 20(8):1929. Published 2019 Apr. 19; Chatterjee A, Xiao H, Schultz P G. Evolution of multiple, mutually orthogonal prolyl-tRNA synthetase/tRNA pairs for unnatural amino acid mutagenesis in Escherichia coli. Proc Natl Acad Sci USA. 2012; 109(37):14841-14846; Thibodeaux GN, Liang X, Moncivais K, et al. Transforming a pair of orthogonal tRNA-aminoacyl-tRNA synthetase from Archaea to function in mammalian cells. PLOS One. 2010; 5(6):e11263. Published 2010 Jun. 22; and Using a Quadruplet Codon to Expand the Genetic Code of an Animal, Zhiyan Xi, Lloyd Davis, Kieran Baxter, Ailish Tynan, Angeliki Goutou, Sebastian Greiss. bioRxiv 2021.07.17.452788).

### Multicistronic Expression Constructs

In example embodiments, at least three qtRNAs can be expressed from a single expression construct. Applicants unexpectedly discovered that multiplex qtRNA decoding is greatly diminished when more than one qtRNA is expressed on separate plasmids and expression from a multicistronic construct greatly improves multiplex decoding. In one example embodiment, at least three orthogonal qtRNAs are expressed from a single expression construct. In certain embodiments, 3, 4, 5 or 6 qtRNAs are expressed from a single expression construct. In certain embodiments, the expression construct is a multicistronic construct. As used herein a “multicistronic construct” or “polycistronic construct” refers to a construct that simultaneously expresses two or more genes (e.g., tRNAs) using a single promoter.

### Multicistronic Templates

A multicistronic construct can be generated de novo or can be derived from an endogenous multicistronic sequence. In certain embodiments, the endogenous multicistronic construct is derived from a multicistronic tRNA operon from an organism (e.g., bacteria). Genes encoding for tRNA are found in polycistronic transcription units in bacteria (see, e.g., Nakajima N, Ozeki H, Shimura Y. Organization and structure of an E. coli tRNA operon containing seven tRNA genes. Cell. 1981; 23(1):239-249). In preferred embodiments, the tRNA operon is derived from a prokaryotic organism. In preferred embodiments, the endogenous operon contains at least three, more preferably, at least 4 tRNAs. Example multicistronic scaffolds are shown in Tables 6 and 7.

Eukaryotic tRNA genes can be arranged in clusters and are not normally found in an operon, however, eukaryotic operons have been found (see, e.g., Blumenthal T. Operons in eukaryotes. Brief Funct Genomic Proteomic 2004 November; 3(3):199-211). Furthermore, polycistronic transcription units are occasionally found in eukaryotes, such as in plants and protozoa (see, e.g., Kruszka K, Barneche F, Guyot R, et al. Plant dicistronic tRNA-snoRNA genes: a new mode of expression of the small nucleolar RNAs processed by RNase Z. EMBO J. 2003; 22(3):621-632; and Nakaar V, Dare A O, Hong D, Ullu E, Tschudi C. Upstream tRNA genes are essential for expression of small nuclear and cytoplasmic RNA genes in trypanosomes. Mol Cell Biol. 1994; 14(10):6736-6742). In example embodiments, a qtRNA operon is generated to be expressed in a eukaryotic cell (e.g., yeast, insect, mammalian, or plant). In one example embodiment, a prokaryotic promoter is replaced with a eukaryotic RNA Pol III promoter. In one example embodiment, a construct is generated that is under control of a Pol III promoter and the at least three qtRNAs are separated by internal ribosome entry sites (IRES), or any sequence capable of re-initiating translational following the stop codon. Additionally, the initial transcript can include sequences that are processed by cleavage and trans-splicing to create monocistronic qtRNAs. For example, the expression of Csy4 endoribonuclease (endo-RNase) to process a transcript containing qtRNAs fused with Csy4-cleavable RNA (see, e.g., Nissim L, Perli S D, Fridkin A, Perez-Pinera P, Lu T K. Multiplexed and programmable regulation of gene networks with an integrated RNA and CRISPR/Cas toolkit in human cells. Mol Cell. 2014; 54(4):698-710). In another example embodiment, tRNA polycistronic operons are used to generate a construct. In one embodiment, a plant or protozoa polycistronic tRNA operon is used in eukaryotic cells other than plants, such as yeast, insect cells or mammalian cells. In one embodiment, a multicistronic construct derived from bacteria is used in eukaryotic cells (e.g., mammalian, insect, or yeast). In one embodiment, any polycistronic operon in plants is used for the construct by replacing any of the polycistronic genes (e.g., snoRNAs) with a qtRNA gene. The tRNA-processing system including RNase P and RNase Z is universal in all living organisms, thus, allowing use of any tRNA polycistronic construct in any cell type. For example, it has been demonstrated that an artificial polycistronic-tRNA-gRNA (PTG) gene derived from plants can be used for multiplex genome editing in human and animal systems based on recognition and cleavage of the tRNAs by RNases P and Z to release the tRNAs and gRNAs (Dong F, Xie K, Chen Y, Yang Y, Mao Y. Polycistronic tRNA and CRISPR guide-RNA enables highly efficient multiplexed genome engineering in human cells. Biochem Biophys Res Commun. 2017; 482(4):889-895; see, also, Xie K, Minkenberg B, Yang Y. Boosting CRISPR/Cas9 multiplex editing capability with the endogenous tRNA-processing system. Proc Natl Acad Sci USA. 2015; 112(11):3570-3575).

In one example embodiment, a multicistronic construct is generated to express qtRNAs in a specific cell type. In an embodiment, the multicistronic construct is derived from a tRNA operon from the organism of the specific cell type. In one example embodiment, the qtRNAs are heterologous to the cell type. In one example embodiment, the qtRNAs are derived from tRNAs specific to the cell type.

### Vectors

In example embodiments, a vector is used to express the multicistronic construct in a cell of interest. In general, and throughout this specification, the term “vector” refers to a nucleic acid molecule capable of transporting another nucleic acid to which it has been linked. Vectors include, but are not limited to, nucleic acid molecules that are single-stranded, double-stranded, or partially double-stranded; nucleic acid molecules that comprise one or more free ends, no free ends (e.g., circular); nucleic acid molecules that comprise DNA, RNA, or both; and other varieties of polynucleotides known in the art. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively-linked. Such vectors are referred to herein as “expression vectors.” Vectors for and that result in expression in a eukaryotic cell can be referred to herein as “eukaryotic expression vectors.” Common expression vectors of utility in recombinant DNA techniques are often in the form of plasmids. There are no limitations regarding the type of vector that can be used. The vector can be a cloning vector, suitable for propagation and for obtaining polynucleotides, gene constructs or expression vectors incorporated to several heterologous organisms. Suitable vectors include prokaryotic expression vectors for use in generating recombinant protein, such as plasmids. Suitable vectors also include eukaryotic expression vectors based on viral vectors (e.g. adenoviruses, adeno-associated viruses as well as retroviruses and lentiviruses), as well as non-viral vectors such as plasmids.

Recombinant expression vectors can comprise a nucleic acid of the invention in a form suitable for expression of the nucleic acid in a host cell, which means that the recombinant expression vectors include one or more regulatory elements, which may be selected on the basis of the host cells to be used for expression, that is operably linked to the nucleic acid sequence to be expressed. Within a recombinant expression vector, “operably linked” is intended to mean that the nucleotide sequence of interest is linked to the regulatory element(s) in a manner that allows for expression of the nucleotide sequence (e.g., in an in vitro transcription/translation system or in a host cell when the vector is introduced into the host cell).

The term “regulatory element” is intended to include promoters, enhancers, internal ribosomal entry sites (IRES), and other expression control elements (e.g., transcription termination signals, such as polyadenylation signals and poly-U sequences). Such regulatory elements are described, for example, in Goeddel, GENE EXPRESSION TECHNOLOGY: METHODS IN ENZYMOLOGY 185, Academic Press, San Diego, Calif. (1990). Regulatory elements include those that direct constitutive or inducible expression of a nucleotide sequence in many types of host cell and those that direct expression of the nucleotide sequence only in certain host cells (e.g., tissue-specific regulatory sequences). Regulatory elements may also direct expression in a temporal-dependent manner, such as in a cell-cycle dependent or developmental stage-dependent manner, which may or may not also be tissue or cell-type specific. In some embodiments, a vector comprises one or more prokaryotic promoters, one or more pol III promoter (e.g., 1, 2, 3, 4, 5, or more pol III promoters), one or more pol II promoters (e.g., 1, 2, 3, 4, 5, or more pol II promoters), one or more pol I promoters (e.g., 1, 2, 3, 4, 5, or more pol I promoters), or combinations thereof. Examples of pol III promoters include, but are not limited to, U6 and H1 promoters. Examples of pol II promoters include, but are not limited to, the retroviral Rous sarcoma virus (RSV) LTR promoter (optionally with the RSV enhancer), the cytomegalovirus (CMV) promoter (optionally with the CMV enhancer) [see, e.g., Boshart et al, Cell, 41:521-530 (1985)], the SV40 promoter, the dihydrofolate reductase promoter, the β-actin promoter, the phosphoglycerol kinase (PGK) promoter, and the EF1α promoter. Also encompassed by the term “regulatory element” are enhancer elements, such as WPRE; CMV enhancers; the R-U5′ segment in LTR of HTLV-I (Mol. Cell. Biol., Vol. 8(1), p. 466-472, 1988); SV40 enhancer; and the intron sequence between exons 2 and 3 of rabbit β-globin (Proc. Natl. Acad. Sci. USA., Vol. 78(3), p. 1527-31, 1981). It will be appreciated by those skilled in the art that the design of the expression vector can depend on such factors as the choice of the host cell to be transformed, the level of expression desired, etc. A vector can be introduced into host cells to thereby produce transcripts, proteins, or peptides, including fusion proteins or peptides, encoded by nucleic acids as described herein (e.g., qtRNAs and/or AARSs). In one example embodiment, two or more of the elements of a system are expressed from the same or different regulatory elements, may be combined in a single vector, with one or more additional vectors providing any components of the system not included in the first vector.

In one example embodiment, the vector is a “plasmid,” which refers to a circular double stranded DNA loop into which additional DNA segments can be inserted, such as by standard molecular cloning techniques.

In one example embodiment, the vector is a viral vector, wherein virally-derived DNA or RNA sequences are present in the vector for packaging into a virus (e.g., retroviruses, replication defective retroviruses, adenoviruses, replication defective adenoviruses, and adeno-associated viruses). Viral vectors also include polynucleotides carried by a virus for transfection into a host cell. Certain vectors are capable of autonomous replication in a host cell into which they are introduced (e.g., episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operably-linked. Such vectors are referred to herein as “expression vectors.” Vectors for and that result in expression in a eukaryotic cell can be referred to herein as “eukaryotic expression vectors.” In another example embodiment, the vector integrates the gene into the cell genome or is maintained episomally.

In one example embodiment, the vector uses a promoter specific to the cell of interest. For example, a Pol III promoter derived from the eukaryotic cell of interest or a bacterial promoter.

In example embodiments, vectors drive expression recombinant protein (e.g., recombinant proteins encoding for one or more quadruplet codons). Expression of proteins in prokaryotes is most often carried out in Escherichia coli with vectors containing constitutive or inducible promoters directing the expression of either fusion or non-fusion proteins. Fusion vectors add a number of amino acids to a protein encoded therein, such as to the amino terminus of the recombinant protein. Such fusion vectors may serve one or more purposes, such as: (i) to increase expression of recombinant protein; (ii) to increase the solubility of the recombinant protein; and (iii) to aid in the purification of the recombinant protein by acting as a ligand in affinity purification. Often, in fusion expression vectors, a proteolytic cleavage site is introduced at the junction of the fusion moiety and the recombinant protein to enable separation of the recombinant protein from the fusion moiety subsequent to purification of the fusion protein. Such enzymes, and their cognate recognition sequences, include Factor Xa, thrombin and enterokinase. Example fusion expression vectors include pGEX (Pharmacia Biotech Inc; Smith and Johnson, 1988. Gene 67: 31-40), pMAL (New England Biolabs, Beverly, Mass.) and pRIT5 (Pharmacia, Piscataway, N.J.) that fuse glutathione S-transferase (GST), maltose E binding protein, or protein A, respectively, to the target recombinant protein. Examples of suitable inducible non-fusion E. coli expression vectors include pTrc (Amrann et al., (1988) Gene 69:301-315) and pET 11d (Studier et al., GENE EXPRESSION TECHNOLOGY: METHODS IN ENZYMOLOGY 185, Academic Press, San Diego, Calif. (1990) 60-89). In some embodiments, a vector is a yeast expression vector. Examples of vectors for expression in yeast Saccharomyces cerivisae include pYepSec1 (Baldari, et al., 1987. EMBO J. 6: 229-234), pMFa (Kuijan and Herskowitz, 1982. Cell 30: 933-943), pJRY88 (Schultz et al., 1987. Gene 54: 113-123), pYES2 (Invitrogen Corporation, San Diego, Calif.), and picZ (InVitrogen Corp, San Diego, Calif.). In some embodiments, a vector drives protein expression in insect cells using baculovirus expression vectors. Baculovirus vectors available for expression of proteins in cultured insect cells (e.g., SF9 cells) include the pAc series (Smith, et al., 1983. Mol. Cell. Biol. 3: 2156-2165) and the pVI, series (Lucklow and Summers, 1989. Virology 170: 31-39). In some embodiments, a vector is capable of driving expression of one or more sequences in mammalian cells using a mammalian expression vector. Examples of mammalian expression vectors include pCDM8 (Seed, 1987. Nature 329: 840) and pMT2PC (Kaufman, et al., 1987. EMBO J. 6: 187-195). When used in mammalian cells, the expression vector's control functions are typically provided by one or more regulatory elements. For example, commonly used promoters are derived from polyoma, adenovirus 2, cytomegalovirus, simian virus 40, and others disclosed herein and known in the art.

In practicing any of the methods disclosed herein, a suitable vector can be introduced to a cell via one or more methods known in the art, including without limitation, microinjection, electroporation, sonoporation, biolistics, calcium phosphate-mediated transfection, cationic transfection, liposome transfection, dendrimer transfection, heat shock transfection, nucleofection transfection, magnetofection, lipofection, impalefection, optical transfection, proprietary agent-enhanced uptake of nucleic acids, and delivery via liposomes, immunoliposomes, virosomes, or artificial virions.

Methods of Increasing qtRNA Decoding Efficiency

Applicants have shown that qtRNA efficiency can be increased such that quadruplet codon decoding is commercially viable or feasible for any quadruplet codon and amino acid (e.g., ncAA). The following methods and schemes can be applied to any qtRNA, including qtRNAs specific for ncAAs.

### Selecting qtRNAs

In example embodiments, a qtRNA is selected for enhancement of efficiency. In certain embodiments, any qtRNA is chosen. In one example embodiment, the qtRNA includes a quadruplet anti-codon loop specific for a desired quadruplet codon. qtRNAs may be selected from qtRNAs developed using natural tRNA frameworks. For example, a quadruplet anti-codon is inserted into a natural tRNA framework. The ability of a qtRNAs to decode a quadruplet codon may be determined by a functional assay. The functional assay may be, or be similar to, assays described for validation further herein.

In one example embodiment, qtRNAs are selected from a library of qtRNAs. In one example embodiment, qtRNAs specific for quadruplet codons are selected using a library-cross-library screen for expression of a quadruplet containing reporter or selectable marker gene. In one embodiment, the first library contains degenerate quadruplet codon libraries at amino acid-specific positions in the reporter gene or selectable marker and the second library contains a degenerate quadruplet anticodon tRNA library to nominate codon-anticodon pairs. In one example embodiment, qtRNA/AARS pairs are selected using a library-cross-library screen for expression of a quadruplet containing reporter or selectable marker gene. In one embodiment, the first library contains degenerate quadruplet codon libraries at amino acid-specific positions in the reporter gene or selectable marker and the second library contains AARSs to nominate qtRNA AARS pairs.

### Evolution of qtRNAs

In example embodiments, qtRNAs with low efficiency (e.g., 6% efficiency), or no detectable decoding activity, are evolved in a process that selects for efficient quadruplet codon decoding. Applicants show for the first time that modification of nucleotides in the entire qtRNA sequence and not just the anticodon loop can increase efficiency of decoding activity, such that qtRNAs are operable in quadruplet codon systems, preferably, multiplex systems having more than one quadruplet codon. In example embodiments, the entire sequence of qtRNAs are evolved in a continuous evolution system or a noncontinuous evolution system that does not require introduction of mutations by a user. In certain embodiments, qtRNAs are evolved based on a M13 bacteriophage system (see, e.g., Bryson D I, Fan C, Guo L T, Miller C, Söll D, Liu D R. Continuous directed evolution of aminoacyl-tRNA synthetases Nat Chem Biol. 2017; 13(12):1253-1260; and Roth T B, Woolston B M, Stephanopoulos G, Liu D R. Phage-Assisted Evolution of Bacillus methanolicus Methanol Dehydrogenase 2. ACS Synth Biol. 2019; 8(4):796-806). Thus, the system for evolution is completely unbiased as to mutations in the entire sequence of the qtRNA. Previous studies randomized bases in anticodon stem-loop to evolve qtRNAs (Wang N, Shang X, Cerny R, Niu W, Guo J. Systematic Evolution and Study of UAGN Decoding tRNAs in a Genomically Recoded Bacteria. Sci Rep. 2016; 6:21898).

### Continuous Evolution

In example embodiments, continuous evolution is used to generate qtRNAs. As used herein “continuous evolution” refers to any method in which evolving genes are subjected to continuous, seamless cycles of mutagenesis and selection. This is in contrast to directed evolution in which mutagenesis and selection are performed in discrete steps that require mutations to be introduced by scientists at every iteration of evolution. One example method of continuous evolution that can be used to generate qtRNAs is phage-assisted continuous evolution (PACE) (see, e.g., Esvelt, K. M., Carlson, J. C. & Liu, D. R. A system for the continuous directed evolution of biomolecules. Nature 472, 499-U550 (2011)). PACE has previously used for the continuous directed evolution of aminoacyl-tRNA synthetases, which is applicable to embodiments relating to generating an AARS specific to an amino acid or ncAA as described herein (see, e.g., Bryson D I, Fan C, Guo L T, Miller C, Söll D, Liu D R. Continuous directed evolution of aminoacyl-tRNA synthetases Nat Chem Biol. 2017; 13(12):1253-1260).

PACE utilizes the continuous infection of E. coli host cells by a modified version of the M13 bacteriophage (Popa S C, Inamoto I, Thuronyi B W, Shin J A. Phage-Assisted Continuous Evolution (PACE): A Guide Focused on Evolving Protein-DNA Interactions. ACS Omega. 2020; 5(42):26957-26966). The mature M13 bacteriophage particle features a rod-shaped protein shell carrying a circular single-stranded phage DNA. The protein shell contains five different phage coat proteins. The majority of the coat is built from more than 2000 copies of phage protein pVIII, while smaller numbers of proteins pIII, pVI, pVII, and pIX are found at the ends of the rod-shaped shell. All coat proteins are essential for the maturation of the M13 phage. Phage protein pIII, which is encoded by phage gene glIl, is essential for phage maturation and infectivity. The infectivity of M13 phage scales with increasing levels of pIII over a range of 2 orders of magnitude. PACE utilizes a mutant M13 bacteriophage whose gIII gene is replaced by that for the protein of interest. Id. The mutant phage is called Selection Phage, SP. Id. Thus, the SP expresses the protein instead of pIII in host E. coli; the SP cannot produce mature phage particles by itself. Id. To complement the SP, gIII is supplied on a separate plasmid in the host E. coli (Accessory Plasmid, AP) as part of a selection system that activates pIII production (the “gIII selection system”) in response to the activity of the protein of interest. Id. SP can only propagate by expressing the protein from phage DNA, followed by expression of gIII that is mediated by the protein's activity. Id. Thus, successful SP propagation is linked to the activity of the protein of interest. Id. SP carrying a mutant protein with enhanced activity will have a fitness advantage over other SP particles, because the enhanced protein activity allows for increased pIII production, thereby increasing offspring production. Id. Over time, SP harboring the coding sequences expressing improved proteins will outcompete others in the population. Id. It is estimated to take one to two years for a lab to complete this workflow from scratch, although the timeline will vary depending on the lab's experience with PACE, the complexities of the protein of interest, and the existence of selection assays that are valid for the protein of interest. Id. Designing and constructing the selection could take from 0.5 to 1.5 years depending on how much troubleshooting is required. Id. Evolving the protein of interest in PACE once the system is set up could take up to a year to perform the actual experiment, interpret the results, and examine fitness-improving mutations. Id.

The present application utilizes a PACE assay developed to evolve qtRNAs and not a protein. In example embodiments, the SP encodes for a qtRNA in place of gIII and the accessory plasmid (AP) encodes gIII comprising one or more quadruplet codons that are required to be decoded by the qtRNA in order to produce functional pIII. The AP can include, 1, 2, 3, 4, 5, or more quadruplet codons in gIII in order to increase stringency of the assay. The quadruplet codons can encode for a permissive or non-permissive amino acid in order to increase stringency of the assay. As used herein, permissive refers to an amino acid site in a protein that can tolerate more than one amino acid at the site and still produce a functional protein. As used herein, non-permissive refers to an amino acid site in a protein that requires the correct amino acid at the site to produce a functional protein. Amino acid sites can be permissive or non-permissive for a ncAA. The ncAA may be based on the correct amino acid and allow a functional protein in a permissive amino acid site. Thus, the site may be permissive to the ncAA and non-permissive to any other amino acid. In example embodiments where a qtRNA specific for a ncAA is evolved the system encodes for an AARS specific for the ncAA and qtRNA. Further, the system includes the ncAA.

In other example embodiments, mutations are enhanced in the evolution system (continuous or noncontinuous) by including one or more agents capable of enhancing the mutation rate. Mutagens may be of physical, chemical or biological origin. In one example embodiment, one or more mutator proteins are expressed in the evolution system. In preferred embodiments, the mutator proteins are small-molecule inducible. In another preferred embodiment. the mutator proteins are expressed from mutagenesis plasmids (MPs). Non-limiting examples of mutator proteins include dnaQ926, a dominant-negative variant of the E. coli DNA Pol III proofreading domain; umuD′, umuC and recA730, which together enable in vivo translesion mutagenesis employing ultraviolet light or chemical mutagens; DNA methylation (dam); the hemimethylated GATC-binding domain SeqA (seqA); dominant-negative variants of mutS, mutL, or mutH; natural protein inhibitors of Ung, ugi and p56; cytidine deaminase cdal; and the emrR transcriptional repressor (see, e.g., Badran A H, Liu D R. Development of potent in vivo mutagenesis plasmids with broad mutational spectra. Nat Commun. 2015; 6:8425). In certain embodiments, the MP is any of MP1, MP2, MP3, MP4, MP5, or MP6, preferably, MP6 (Id.). Other non-limiting agents include nucleobase analogues, ionizing radiation, such as X-rays, gamma rays and alpha particles, ultraviolet radiation, reactive oxygen species (ROS), deaminating agents, alkylating agents, and intercalating agents, such as ethidium bromide and proflavine.

### Noncontinuous Evolution

In example embodiments, noncontinuous evolution is used to generate qtRNAs. The noncontinuous evolution scheme utilizes the same components as for continuous evolution above, however, the system uses subculture into fresh media instead of media continuously flowing from a Chemostat into a Lagoon (Popa S C, Inamoto I, Thuronyi B W, Shin J A. Phage-Assisted Continuous Evolution (PACE): A Guide Focused on Evolving Protein-DNA Interactions. ACS Omega. 2020; 5(42):26957-26966). A non-limiting example of noncontinuous evolution is phage-assisted noncontinuous evolution (PANCE) (see, e.g., Roth TB, Woolston BM, Stephanopoulos G, Liu D R. Phage-Assisted Evolution of Bacillus methanolicus Methanol Dehydrogenase 2. ACS Synth Biol. 2019; 8(4):796-806).

### Validation of qtRNA

In example embodiments, qtRNAs that have been selected through evolution are further validated in a cellular system or translation system. As used herein, validation refers to assaying for qtRNA function in a non-phage system or any other assay than used in continuous or noncontinuous evolution. The validation can utilize an IVT system or a cellular system. The cellular or IVT system may be provided ncAAs and an AARS capable of charging the qtRNA.

The validation system may utilize a reporter gene or selectable marker to determine qtRNA activity. The reporter gene or selectable marker gene may include one or more quadruplet codons at one or more permissive or nonpermissive amino acid sites, wherein the reporter gene or selectable marker is functional only if the one or more quadruplet codons are decoded by the qtRNA. In one example embodiment, processivity is determined by decoding two or more, 2, 3, 4, 5, or 6 or more, quadruplet codons in a row (i.e., adjacent). In one example embodiment, two markers are separated by a linker where the linker comprises one or more adjacent quadruplet codons to test for expression of both markers.

Nonlimiting selectable markers or reporter genes are green fluorescent protein (e.g., sfGFP), enhanced green fluorescent protein (EGFP), red fluorescent protein (RFP), blue fluorescent protein (BFP), cyan fluorescent protein (CFP), yellow fluorescent protein (YFP), miRFP (e.g., miRFP670, see, e.g., Shcherbakova, et al., Nat Commun. 2016; 7: 12405), mCherry, tdTomato, DsRed-Monomer, DsRed-Express, DSRed-Express2, DsRed2, AsRed2, mStrawberry, mPlum, mRaspberry, HcRed, E2-Crimson, mOrange, mOrange2, mBanana, ZsYellow1, TagBFP, mTagBFP2, Azurite, EBFP2, mKalama1, Sirius, Sapphire, T-Sapphire, ECFP, Cerulean, SCFP3A, m Turquoise, mTurquoise2, monomelic Midoriishi-Cyan, TagCFP, niTFP1, Emerald, Superfolder GFP, Monomeric Azami Green, TagGFP2, mUKG, mWasabi, Clover, mNeonGreen, Citrine, Venus, SYFP2, TagYFP, Monomeric Kusabira-Orange, mKOk, mK02, mTangerine, mApple, mRuby, mRuby2, HcRed-Tandem, mKate2, m. Neptune, NiFP, mkeima Red, LSS-mKate1, LSS-mKate2, mBeRFP, PA-GFP, PAmCherryl, PATagRFP, TagRFP6457, IFP1.2, iRFP, Kaede (green), Kaede (red), KikGR1 (green), KikGR1 (red), PS-CFP2, mEos2 (green), mEos2 (red), mEos3.2 (green), mEos3.2 (red), PSmOrange, Dronpa, Dendra2, Timer, AmCyan1, glutathione-S-transferase (GST), horseradish peroxidase (HRP), chloramphenicol acetyltransferase (CAT), beta-galactosidase (LacZ), beta-glucuronidase, luciferase (e.g., LuxAB), or a combination thereof. In certain embodiments, the detectable marker is a cell surface marker. In other instances, the cell surface marker is a marker not normally expressed on the cells, such as a truncated nerve growth factor receptor (tNGFR), a truncated epidermal growth factor receptor (tEGFR), CD8, truncated CD8, CD19, truncated CD19, a variant thereof, a fragment thereof, a derivative thereof, or a combination thereof. Additional nonlimiting selectable markers or reporter genes include drug resistance genes. In preferred embodiments, selectable markers or reporter genes are detected by a fluorescence or luminescence reader. Efficiency of decoding can be determined by comparing expression of the selectable markers or reporter genes to the expression of the selectable markers or reporter genes in a construct having a wildtype triplet codon.

In example embodiments, the cellular system used for validation is mutated to have decreased or abolished expression of a translation release factor. A release factor is a protein that allows for the termination of translation by recognizing the termination codon or stop codon in an mRNA sequence. There are two classes of release factors. Class 1 release factors recognize stop codons; they bind to the A site of the ribosome in a way mimicking that of tRNA, releasing the new polypeptide as it disassembles the ribosome. Class 2 release factors are GTPases that enhance the activity of class 1 release factors. Class 2 release factors help the class 1 RF to dissociate from the ribosome. Bacterial release factors include RF1, RF2, and RF3 (or PrfA, PrfB, PrfC in the “peptide release factor” gene nomenclature). RF1 and RF2 are class 1 RFs: RF1 recognizes UAA and UAG while RF2 recognizes UAA and UGA. RF3 is the class 2 release factor. Eukaryotic and archaeal release factors are similar, with the naming changed to “eRF” for “eukaryotic release factor” and vice versa. a/eRF1 can recognize all three stop codons, while eRF3 (archaea use EF-1α instead) works just like RF3. In preferred embodiments, a class I release factor is mutated, more preferably, RF1.

In example embodiments, the cellular system is an eukaryotic or prokaryotic systems, such as bacteria, insect cells, yeast, mammalian cells or plant cells.

Use of Evolved qtRNAS or Multicistronic Constructs

### Cells

In an example embodiment, the present invention provides for cells that express a quadruplet encoding system (e.g., one or more qtRNAs and, optionally, one or more AARS). Cells engineered to express qtRNAs can be used to generate protein in many applications. In certain embodiments, cells expressing qtRNAs are also engineered to express aminoacyl-qtRNA synthetases that load the correct amino acid or ncAA on the qtRNA. A variety of cells can be used in certain methods, including, for example, a bacterial cell, a yeast cell, an insect cell, a mammalian cell (e.g., a human cell or a non-human mammal cell), and a plant cell. In one embodiment, the cell is an E. coli cell. In certain embodiments, the ncAA can be provided by directly contacting the cell with the ncAA, for example, by applying a solution of the ncAA to the cell in culture. The ncAA can also be provided by introducing one or more additional nucleic acid construct(s) into the cell, wherein the additional nucleic acid construct(s) encodes one or more ncAA synthesis proteins that are necessary for synthesis of the desired ncAA.

### In Vitro Translation Systems

In an example embodiment, the present invention provides for in vitro translation system (IVT) comprising a quadruplet encoding system. In vitro translation systems facilitate the biosynthesis of recombinant proteins without using intact cells. Example IVT platforms have been described, including purified translation systems and the extract-based systems (see, e.g., Hammerling M J, Krüger A, Jewett M C. Strategies for in vitro engineering of the translation machinery. Nucleic Acids Res. 2020; 48(3):1068-1083). Each system can incorporate qtRNAs of the present invention. In certain embodiments, the IVT system includes aminoacyl-qtRNA synthetase(s) that charges the qtRNA(s) with the correct amino acid or ncAA. A variety of cell lysates can be used in IVT, including, for example, a bacterial cell lysate, a yeast cell lysate, an insect cell lysate, a mammalian cell lysate (e.g., a human cell or a non-human mammal cell), and a plant cell lysate. In one embodiment, the cell is an E. coli cell lysate. In certain embodiments, the ncAA can be provided by directly contacting the IVT system with the ncAA, for example, by directly adding the ncAA to the IVT system.

### Producing Recombinant Proteins

In example embodiments, recombinant proteins can be generated using an expanded genetic code (e.g., by incorporating ncAAs, qtRNAs and, optionally, AARSs). Commonly used recombinant protein production systems include those derived from bacteria (see, e g., Baneyx F (October 1999). “Recombinant protein expression in Escherichia coli”. Current Opinion in Biotechnology. 10 (5): 411-21; and Jia B, Jeon CO. High-throughput recombinant protein expression in Escherichia coli: current status and future perspectives. Open Biol. 2016; 6(8):160196. doi:10.1098/rsob.160196), yeast (see, e.g., Malys N, Wishart J A, Oliver S G, McCarthy J E (2011). “Protein production in Saccharomyces cerevisiae for systems biology studies”. Methods in Systems Biology. Methods in Enzymology. 500. pp. 197-212), baculovirus/insect (see, e.g., Kost T A, Condreay J P, Jarvis D L (May 2005). “Baculovirus as versatile vectors for protein expression in insect and mammalian cells”. Nature Biotechnology. 23 (5): 567 75; and Chambers A C, Aksular M, Graves L P, Irons S L, Possee R D, King LA. Overview of the Baculovirus Expression System. Curr Protoc Protein Sci. 2018; 91:5.4.1-5.4.6), mammalian cells (see, e.g., Dyson M R. Fundamentals of Expression in Mammalian Cells. Adv Exp Med Biol. 2016; 896:217-224; and Hunter M, Yuan P, Vavilala D, Fox M. Optimization of Protein Expression in Mammalian Cells. Curr Protoc Protein Sci. 2019; 95(1): e77), and filamentous fungi such as Myceliophthora thermophila (Visser H, Joosten V, Punt P J, Gusakov A V, Olson P T, Joosten R, et al. (June 2011). “Development of a mature fungal technology and production platform for industrial enzymes based on a Myceliophthora thermophila isolate, previously known as Chrysosporium lucknowense C1”. Industrial Biotechnology. 7 (3): 214-223). Baculovirus-infected insect cells (e.g., Sf9, Sf21, High Five strains) or mammalian cells (e.g., HeLa, HEK 293) allow production of glycosylated or membrane proteins that cannot be produced using fungal or bacterial systems.

### Generating Antibodies

In certain embodiments, qtRNAs can be incorporated into antibodies to modify and enhance the function of the antibodies. For example, the sequences encoding antibodies can include one or more quadruplet codons for the site specific incorporation of ncAAs. Methods of producing recombinant antibodies is known in the art. Mammalian cell lines such as Chinese hamster ovary cells are commonly used as hosts for mAb production, but the process is relatively expensive (Farid SS. Established bioprocesses for producing antibodies as a basis for future planning. Adv Biochem Eng Biotechnol. 2006; 101:1-42). Alternative platforms use plant and microbial expression systems (Potgieter T I, Cukan M, Drummond J E, et al. Production of monoclonal antibodies by glycoengineered Pichia pastoris. J Biotechnol. 2009; 139(4):318-325; Li H, Sethuraman N, Stadheim T A, et al. Optimization of humanized IgGs in glycoengineered Pichia pastoris. Nat Biotechnol. 2006; 24(2):210-215; and Cox K M, Sterling J D, Regan J T, et al. Glycan optimization of a human monoclonal antibody in the aquatic plant Lemna minor. Nat Biotechnol. 2006; 24(12):1591-1597). Of these alternative platforms, yeast-based approaches are regarded as a compelling alternative to mammalian cell culture because of their possibly higher titers, low-cost and scalable fermentation process, and low risk for human pathogenic virus contamination (see, e.g., Liu C P, Tsai T I, Cheng T, et al. Glycoengineering of antibody (Herceptin) through yeast expression and in vitro enzymatic glycosylation. Proc Natl Acad Sci USA. 2018; 115(4):720-725). In example embodiments, any production system known in the art can be modified to express qtRNAs and, optionally, AARSs capable of charging the qtRNAs.

### Directed or Continuous Evolution

In certain embodiments, qtRNAs can be incorporated into a directed, noncontinuous or continuous evolution system, such as phage display, yeast display, ribosome display, PACE, or PANCE. In an example embodiment, the evolution of proteins can be increased using an expanded genetic code. In an example embodiment, quadruplet codons are incorporated into a mRNA sequence at specific residues that are important for a function to be evolved. In certain embodiments, multicistronic constructs are used to express multiple qtRNAs capable of decoding multiple quadruplet codons.

In certain embodiments, phage display systems can utilize qtRNAs to expand the genetic code. Phage display is a widely used method for in vitro protein evolution and can be used for finding new ligands (enzyme inhibitors, receptor agonists and antagonists) to target proteins, in particular, phage display of antibody libraries has become a powerful method for both studying the immune response as well as a method to rapidly select and evolve human antibodies for therapy (see, e.g., Lunder M, Bratkovic T, Doljak B, Kreft S, Urleb U, Strukelj B, Plazar N (November 2005). “Comparison of bacterial and phage display peptide libraries in search of target-binding motif”. Appl. Biochem. Biotechnol. 127 (2): 125-31; Bratkovic T, Lunder M, Popovic T, Kreft S, Turk B, Strukelj B, Urleb U (July 2005). “Affinity selection to papain yields potent peptide inhibitors of cathepsins L, B, H, and K”. Biochem. Biophys. Res. Commun. 332 (3): 897-903; Hufton S E, Moerkerk P T, Meulemans E V, de Bruine A, Arends J W, Hoogenboom H R (December 1999). “Phage display of cDNA repertoires: the pVI display system and its applications for the selection of immunogenic ligands”. J. Immunol. Methods. 231 (1-2): 39-51; and Løset GÅ, Berntzen G, Frigstad T, Pollmann S, Gunnarsen K S, Sandlie I (12 Jan. 2015). “Phage Display Engineered T Cell Receptors as Tools for the Study of Tumor Peptide-MHC Interactions”. Frontiers in Oncology. 4 (378): 378).

In certain embodiments, yeast display systems can utilize qtRNAs to expand the genetic code (see, e.g., McMahon, C. et al. Yeast surface display platform for rapid discovery of conformationally selective nanobodies. Nat. Struct. Mol. Biol. 25, 289-296 (2018); Boder, E. T. & Wittrup, K. D. Yeast surface display for screening combinatorial polypeptide libraries. Nat. Biotechnol. 15, 553-557 (1997).

In certain embodiments, cell free ribosome display systems can utilize qtRNAs to expand the genetic code (see, e.g., Hanes, J. & Pluckthun, A. In vitro selection and evolution of functional proteins by using ribosome display. Proc. Natl. Acad. Sci. U.S.A. 94, 4937-4942 (1997); Hanes, J., Schaffitzel, C., Knappik, A. & Plückthun, A. Picomolar affinity antibodies from a fully synthetic naive library selected and evolved by ribosome display. Nat. Biotechnol. 18, 1287-1292 (2000); and He, M. & Taussig, M. J. Ribosome display: Cell-free protein display technology Briefings Funct. Genomics Proteomics 1, 204-212 (2002))

Further embodiments are illustrated in the following Examples which are given for illustrative purposes only and are not intended to limit the scope of the invention.

### EXAMPLES

### Example 1—Multiplex Suppression of Quadruplet Codons via tRNA Directed Evolution

Applicants developed frameworks for the discovery and characterization of amino-acid specific qtRNAs, finding that anticodon replacement in many Escherichia coli (E. coli) tRNA scaffolds can yield functional, but often inefficient, qtRNAs. To do this, Applicants first created a system to probe quadruplet codon-anticodon interactions by nominating amino acid-specific positions in β-galactosidase (lacZ), yielding functional qtRNAs for many of the canonical amino acids with low suppression efficiencies (up to η=6%). To improve qtRNA efficiency, Applicants leveraged phage-assisted continuous evolution (qtRNA-PACE) to rapidly evolve variants with suppression efficiencies up to 40% under kinetic conditions and without genome modifications. The most efficient qtRNAs perform on par with traditional amber suppression in cellular translation assays, can effectively compete with cellular release factors for codon decoding, show amino-acid specific aminoacylation profiles in most cases, and allow for processive translation of up to six adjacent quadruplet codons. Using these variants, Applicants showcase the selective translation of a model fluorescent reporter protein using four mutually orthogonal qtRNA-quadruplet codon pairs. Collectively, the findings establish orthogonal qtRNAs for 4 of 20 canonical amino acids that can work in concert for the first time, and support the possibility of generating comprehensive, quadruplet-codon decoding reagents for efficient use in living cells.

### Results

A General Framework for qtRNA Discovery and Characterization

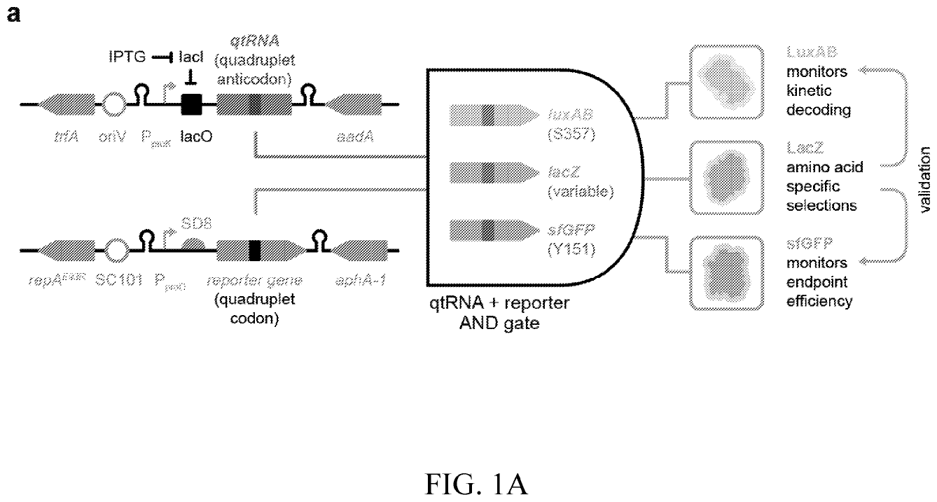

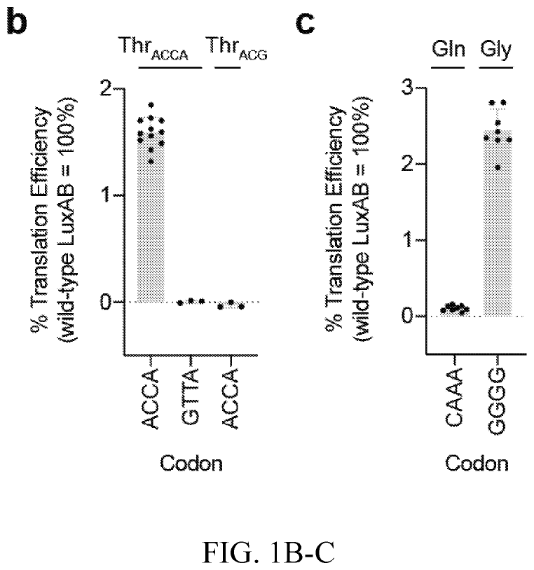

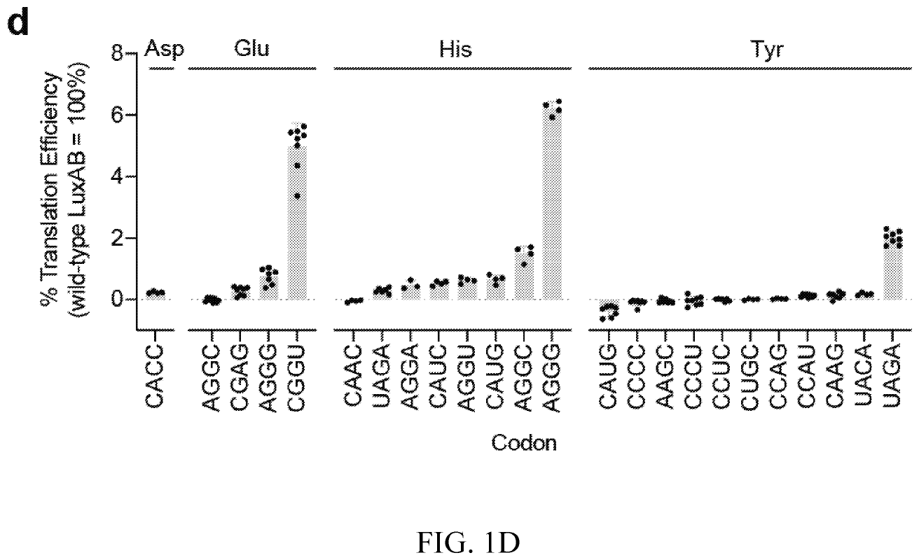

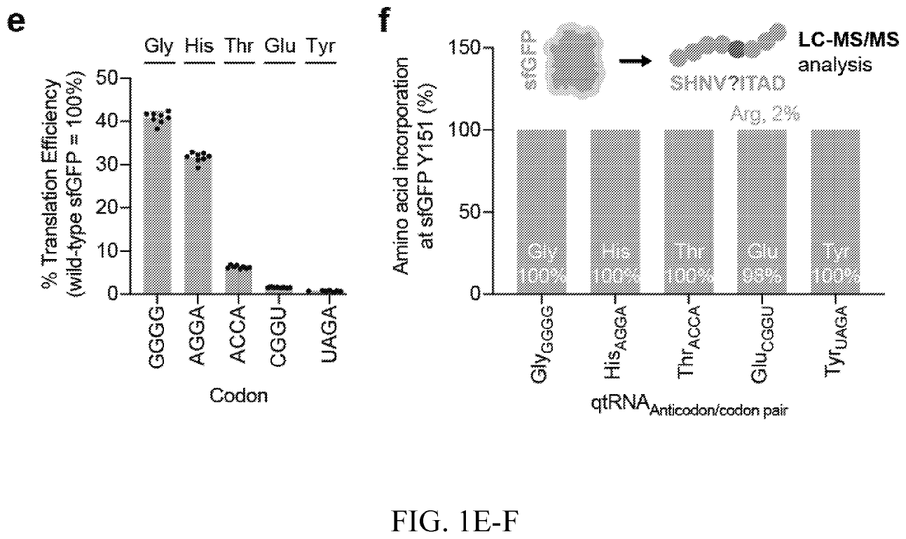

To effectively monitor quadruplet decoding for future engineering efforts, Applicants first created a frameshift reporter by introducing a quadruplet codon into the covalently-linked bacterial luciferase LuxAB^{35, 36} gene (FIG. 1a). Compared to the canonically used superfolder GFP (sfGFP), LuxAB shows a greater dynamic range which may sensitize the detection capabilities and enable the quantification of poorly active qtRNAs³⁵. In addition, Applicants monitored luminescence signals kinetically to assess qtRNA-dependent toxicity, and quantified activity using luminescence at a standard density (OD₆₀₀) to account for differential growth rates.

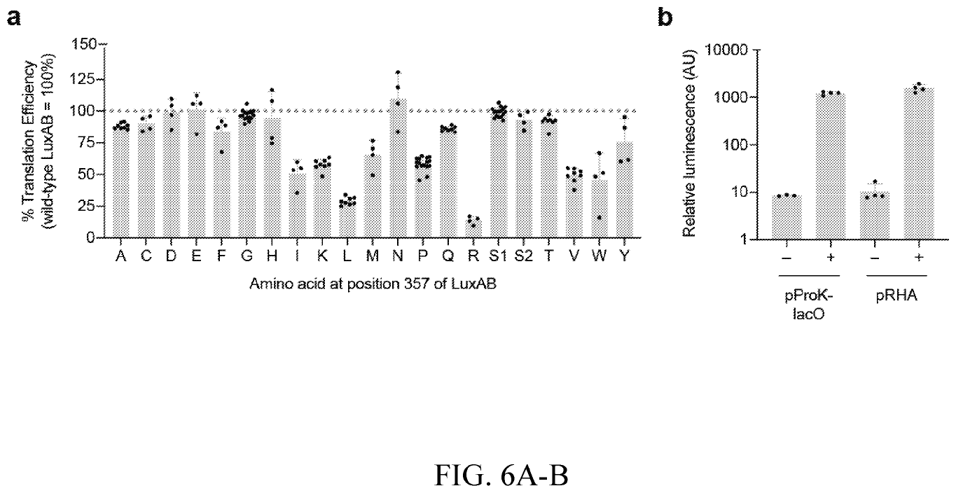

Failure to decode a quadruplet codon in this reporter would lead to premature translation termination, whereas successful decoding yields full-length LuxAB and a corresponding luminescence increase. Applicants confirmed that position 357 of LuxAB is tolerant to most amino acid substitutions (FIG. 6a), and used it as the basis for all cellular reporter assays. To control qtRNA abundance, Applicants designed an expression plasmid that integrates the commonly used proK promoter³⁷ alongside a lacO operator to afford IPTG dependence. This yielded a comparable dynamic range to established inducible promoters (FIG. 6b). Using this sensor, Applicants confirmed that luminescence relies on the presence of a qtRNA and correct codon-anticodon interaction (FIG. 1b), and validated it using three previously reported qtRNAs (FIG. 1b-c, Table 1). In all data, Applicants unify the nomenclature by referring to qtRNAs as qtRNA scaffold codon; e.g., qtRNA^{Tyr}_{UAGA} is a tyrosine tRNA scaffold containing a 5′-UCUA-3′ anticodon that should decode the cognate UAGA quadruplet codon in an mRNA.

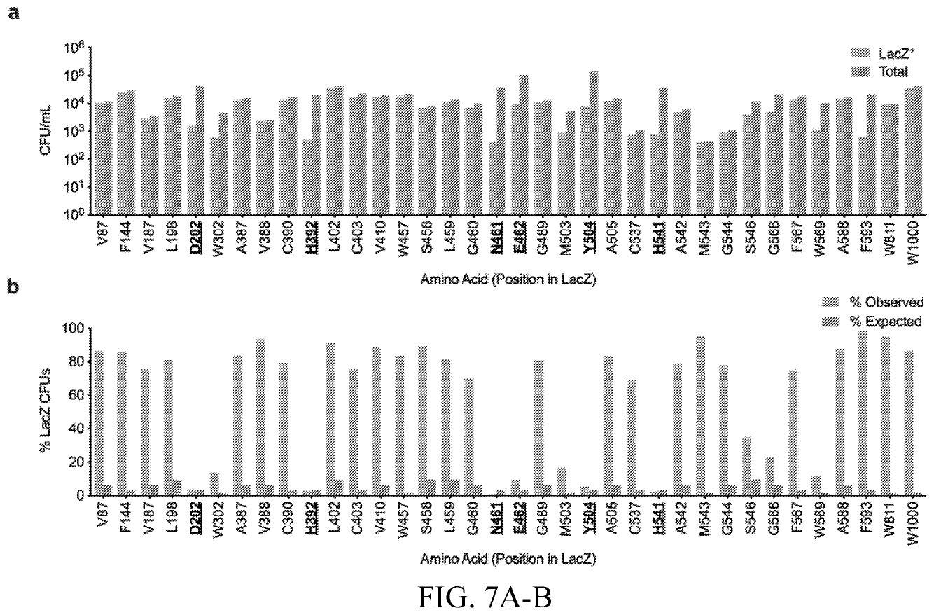

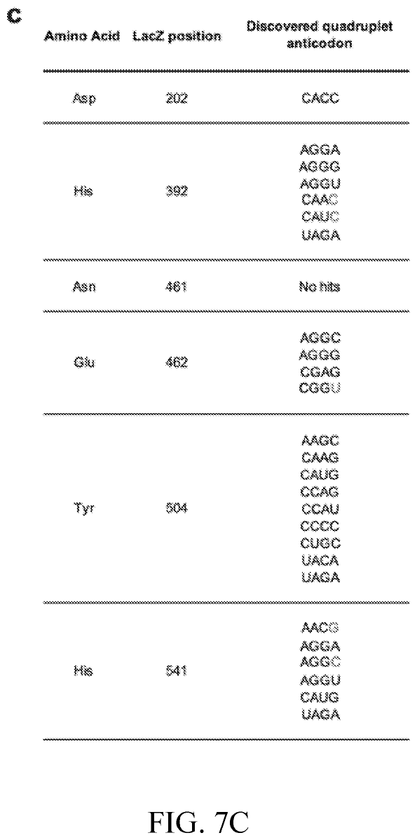

Applicants note that single base changes to tRNAs are known to affect their suppression activities³⁸ and aminoacylation spectra^{28, 39-42}. In particular, tRNA anticodons often serve as identity elements for cognate aminoacyl-tRNA synthetases^{39. 43-46}, suggesting that anticodon engineering may alter charging fidelity. To explore novel quadruplet codon/anticodon interactions without inadvertently altering amino acid identity, Applicants sought to generate a cellular reporter that may serve as the basis for selection in an amino acid-specific manner (FIG. 1a). Using E. coli β-galactosidase (lacZ), Applicants used a degenerate library approach to first confirm that amino acids occupying several positions (D202, H392, N461, E462, Y504, and H541) are absolutely necessary for enzymatic activity (FIG. 7a,b)⁴⁷⁻⁵¹. Next, Applicants carried out library-cross-library selections to identify functional and putatively amino-acid specific qtRNAs. Degenerate quadruplet codon libraries at amino acid-specific positions in lacZ were co-transformed with a degenerate quadruplet anticodon tRNA library to nominate codon-anticodon pairs for future investigation (FIG. 7c). In all cases, representative natural tRNA scaffolds were chosen from the E. coli genome for analysis in these selections (see Methods, Table 2).

Applicants identified qtRNAs that rely on a combination of previously described codon-anticodon interactions, as well as combinations that have not yet been reported (FIG. 1d, FIG. 7c). Notably, codon-anticodon pairs derived from lacZ selections did not always yield measurable luminescence activity upon supplementary validation in the LuxAB reporter system. This may suggest that at least some selection-derived qtRNAs can suffer from context-dependent effects⁵²⁻⁵⁵. Nonetheless, many qtRNAs showed robust decoding efficiencies when assayed in the LuxAB reporter assay, reaching up to η=6% (FIG. 1d).

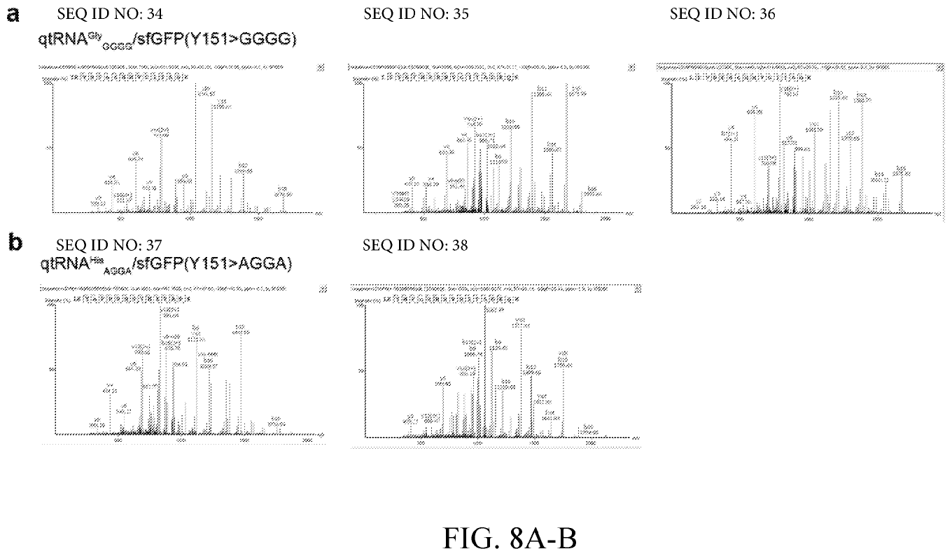

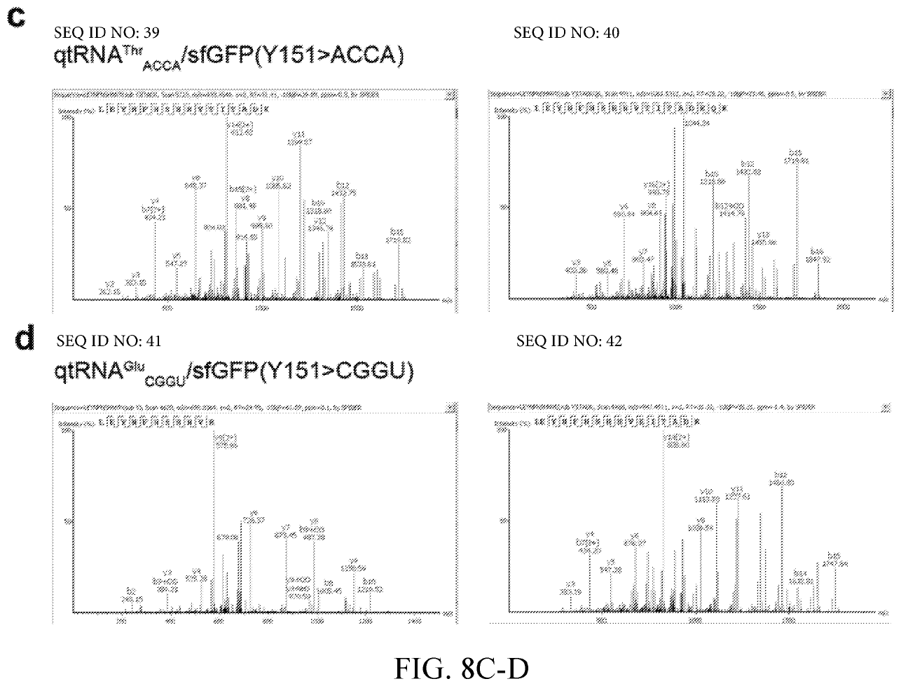

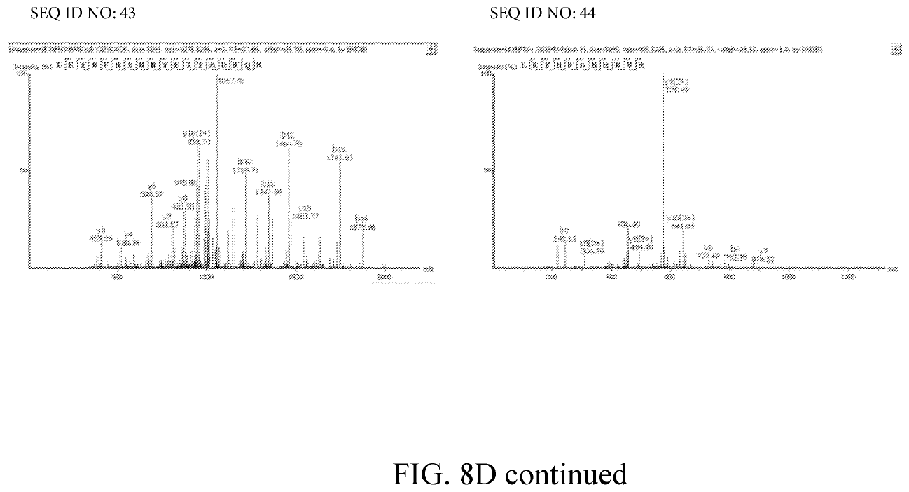

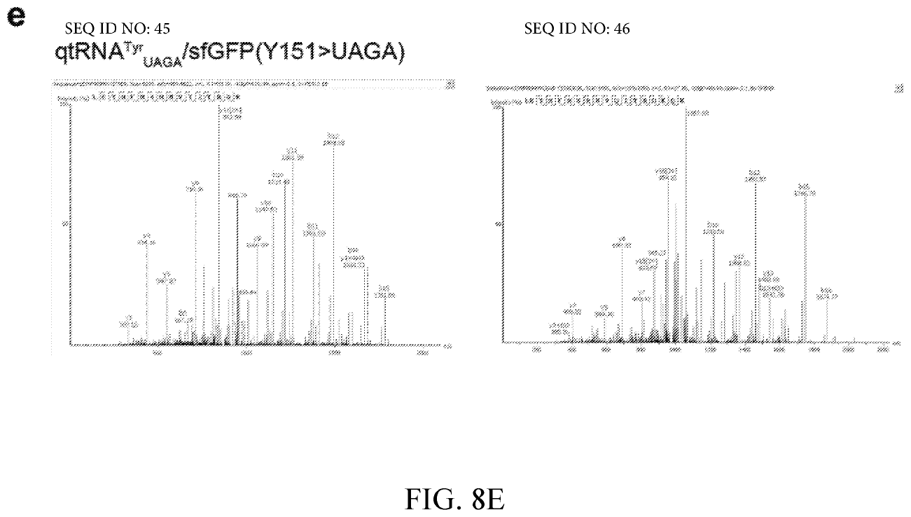

In agreement with these data, the most functional qtRNAs show robust suppression of the cognate quadruplet codon at permissive residue Y151 in sfGFP⁵⁶ (FIG. 1e). Mass spectrometry of sfGFP confirmed the expected amino acid profile at that position following qtRNA-dependent protein translation in the majority of cases (FIG. 1f, FIG. 8). Cumulatively, this approach independently identified 24 total qtRNAs (FIG. 7c), of which all are novel based on prior reports, highlighting that amino acid-specific selections can be used to nominate qtRNAs for effective quadruplet codon translation.

Continuous Directed Evolution of UAGA-Decoding qtRNAs

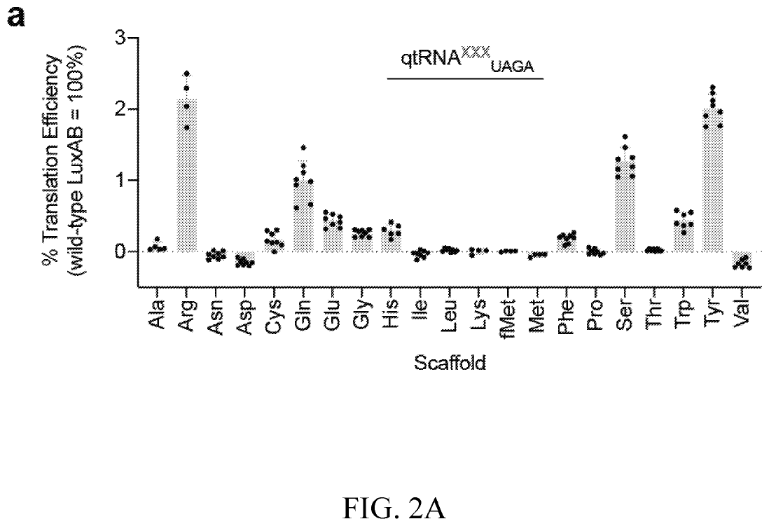

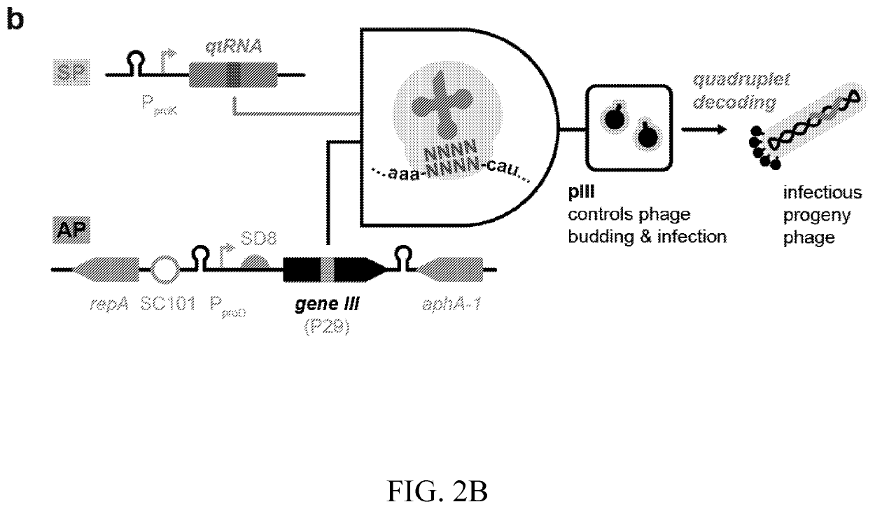

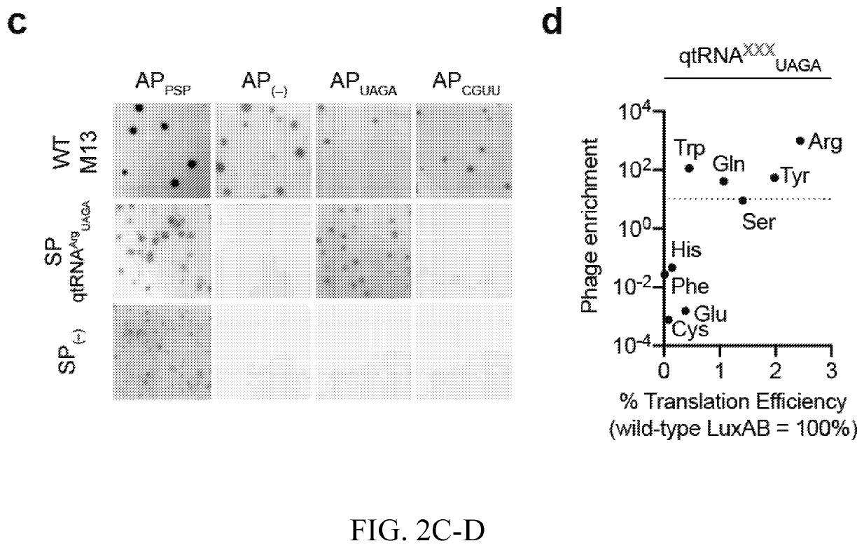

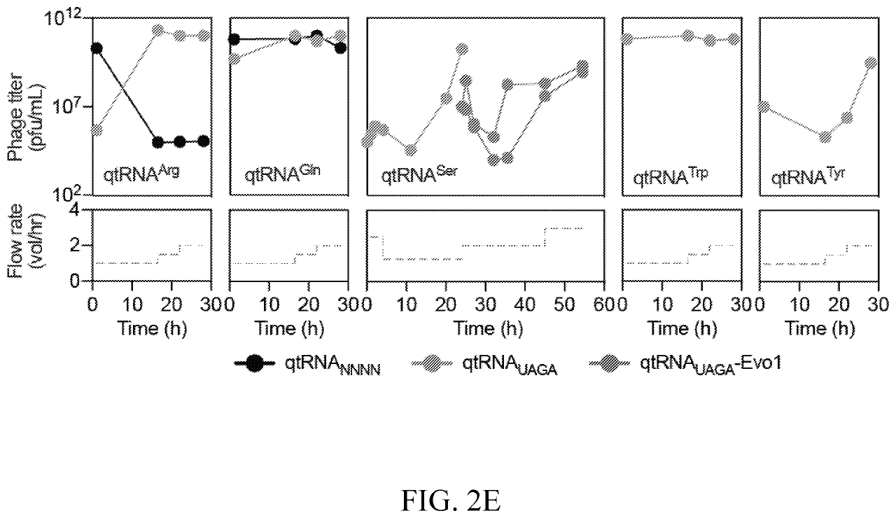

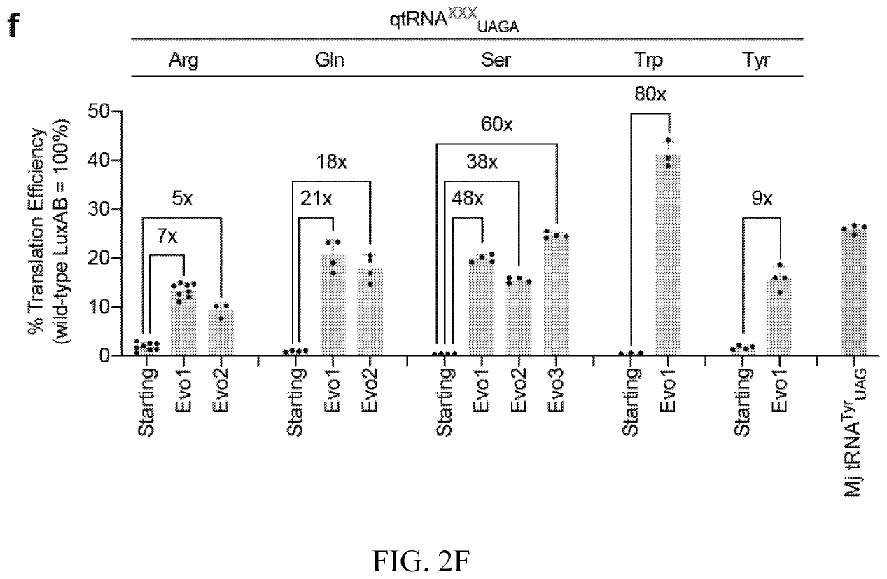

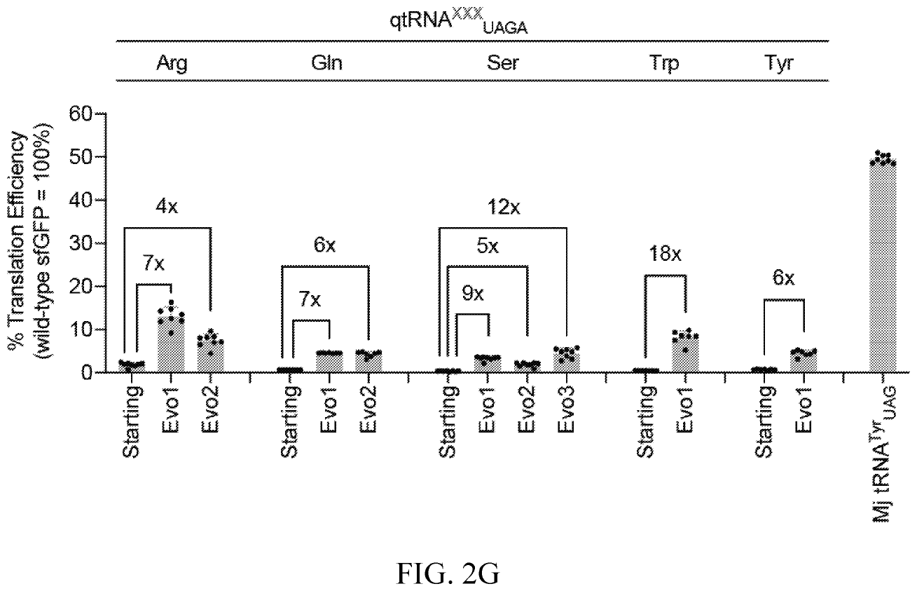

Among the most functional qtRNAs identified through the lacZ library-cross-library selection, Applicants noted that the qtRNA^{Tyr}_{UAGA} and qtRNA^{His}_{UAGA} showed particularly low activity (FIG. 1d). As UAGA-decoding qtRNAs repurpose the low-usage UAG stop codon⁵⁷, they may experience competition with release factor 1 (RF1) for efficient suppression^{33, 58, 59}. To further explore the efficiency of UAGA-decoding, Applicants chose a unique E. coli elongator tRNA for each of the 20 canonical amino acids alongside an initiator tRNA Met (see Methods, Table 2) to serve as scaffolds for qtRNA engineering. In each scaffold, Applicants substituted the anticodon with the sequence 5′-UCUA-3′ to enable UAGA quadruplet codon decoding. Applicants characterized the quadruplet translation efficiency of the 21 engineered UAGA-decoding qtRNAs, finding that nearly half of these qtRNAs (10/21) demonstrated modest UAGA decoding activity compared to triplet decoding (η≤2.5%; FIG. 2a). Universally, the newly engineered UAGA qtRNAs did not cause appreciable host fitness defects (Table 3).

Whereas RF1 deletion can improve UAGA decoding, the resultant strains can show significant fitness defects in rich media⁵⁹, spontaneous reversions to correct genomic instabilities⁶⁰, and low amino acid incorporation fidelity at targeted UAG codons⁶¹. To circumvent these limitations, Applicants hypothesized that supplementary qtRNA modifications may improve UAGA quadruplet codon translation efficiency. Indeed, tRNA scaffold mutations, with a particular focus on stem engineering, can play a key role in the development of host-tolerated and efficient orthogonal tRNAs⁶². These may occur via optimization of scaffold-anticodon compatibility^{63, 64}, by improving qtRNA interactions that were affected by anticodon engineering⁶⁵, or through enhanced competition with RF1.

To improve qtRNA activities in an unbiased manner, Applicants developed a directed evolution platform based on phage-assisted continuous evolution (PACE)⁶⁶ (FIG. 2b). Briefly, a qtRNA is encoded on a selection phage (SP) in place of the M13 bacteriophage minor coat gene gIII (translated to pIII). Following infection, the qtRNA may suppress a quadruplet codon in gIII encoded by an accessory plasmid (AP), resulting in complementation of the gIII-deficient SP and robust progeny propagation. To enhance qtRNA genetic diversity, cellular mutation rates are enhanced by overexpression of mutagenesis plasmid (MP6)-borne mutator proteins⁶⁷.

Applicants introduced a single quadruplet codon at the permissive residue P29 of pIII³⁵ in the AP, thereby generating an amino acid-independent selection for all qtRNAs. Applicants validated this system by challenging E. coli cells bearing APs encoding a CGUU or UAGA quadruplet codon in pIII with SPs encoding or lacking the corresponding qtRNA^{Arg}_{UAGA}, the most active UAGA variant. SP-qtRNA^{Arg}_{UAGA}showed robust translation of pIII (visualized as viral plaques) using AP_{UAGA} but not AP_{CGUU}, and SPs lacking a qtRNA did not show any visible plaques (FIG. 2c). Applicants extended this analysis to additional UAGA qtRNAs, finding a strong correlation between luciferase output and SP propagation in liquid culture (FIG. 2d).

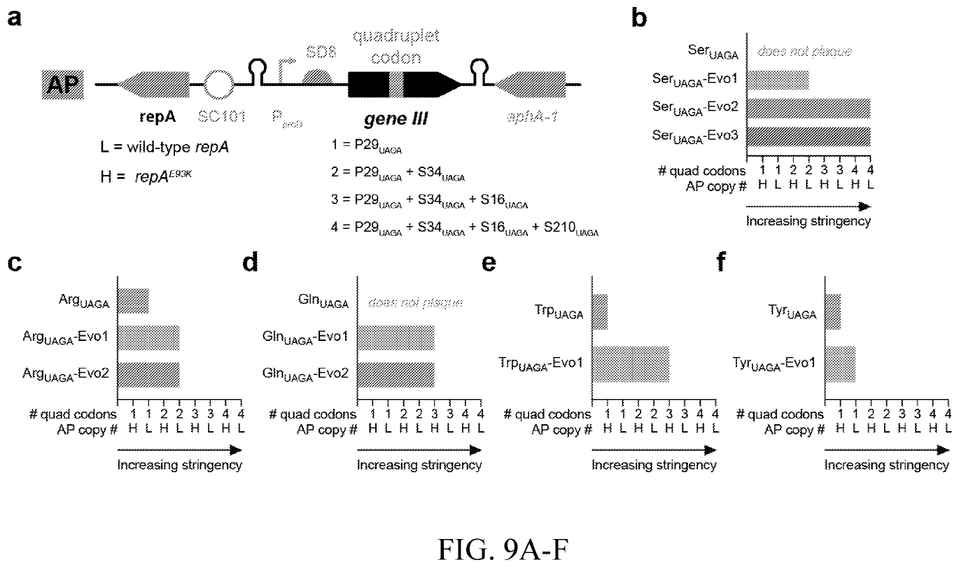

Using this system, Applicants evolved the top five engineered qtRNAs (qtRNA^{Gln}_{UAGA}, qtRNA^{Arg}_{UAGA}, qtRNA^{Ser}_{UAGA}, qtRNA^{Trp}_{UAGA}, and qtRNA^{Tyr}_{UAGA}) and two degenerate qtRNA libraries (qtRNA^{Gln}_{NNNN} and qtRNA^{Arg}_{NNNN}) for 28 hours of qtRNA-PACE using AP_{1×UAGA} and MP6⁶⁷ (FIG. 2e). By the end of this short campaign, all qtRNAs retained or discovered the expected 5′ UCUA 3′ anticodon, and evolved qtRNAs showed improved SP propagation activities compared to their starting counterparts (FIG. 9).

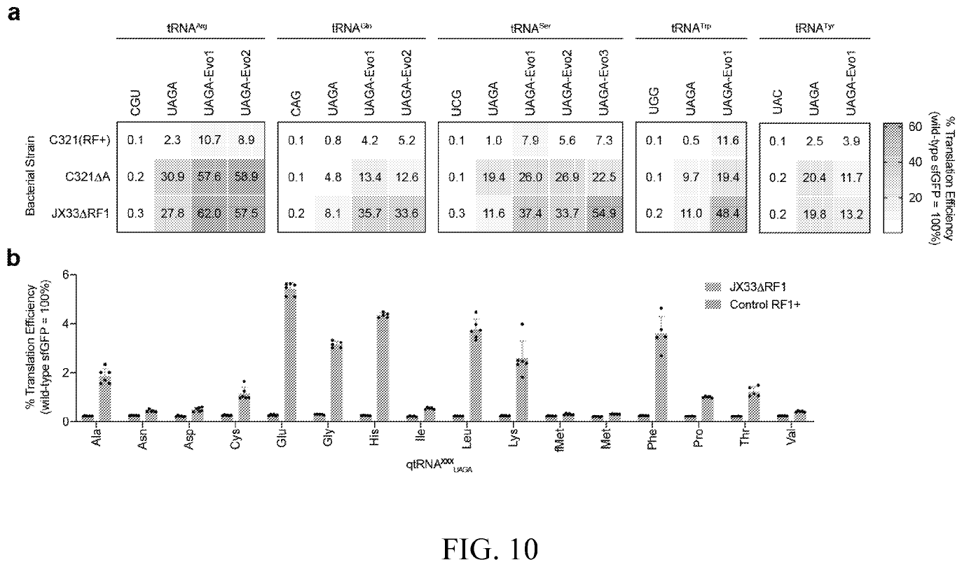

Applicants subcloned each unique evolved qtRNAs into expression plasmids and assayed quadruplet codon translation efficiencies using the LuxAB reporter, finding that all qtRNAs had improved by nearly an order of magnitude in quadruplet codon decoding efficiency (10%<η<40%) without corresponding increases in toxicity (FIG. 2f,g; Table 3). Applicants note qtRNA^{Ser}_{UAGA}evolution yielded a previously characterized double mutant (qtRNA^{Ser}_{UAGA}-Evo1; C32A, A38C) with improved quadruplet codon suppression activity⁸. Evolution of qtRNA^{Ser}_{UAGA}-Evo1 SP for an additional 30 h of qtRNA-PACE (FIG. 2e) using the more stringent AP_{3×UAGA} and MP6 gave rise to qtRNA^{Ser}_{UAGA}-Evo2 and qtRNA^{Ser}_{UAGA}-Evo3. Both variants supported SP propagation using APs that encode up to four UAGA codons (FIG. 9) and moderately improved activities in the LuxAB reporter (FIG. 2f,g).

Whereas qtRNA-dependent translation has historically been less efficient than amber (UAG) suppression strategies, the newly evolved variants showed efficiencies comparable to the commonly used wild-type Methanocaldococcus jannaschii TyrRS-tRNA^{Tyr}_{UAG} pair⁶⁸ when validated in the luciferase reporter (FIG. 2f), and a slight reduction in efficiency when compared using the sfGFP reporter (FIG. 2g). Further assaying engineered and evolved UAGA qtRNAs against an sfGFP reporter incorporating a UAGA codon at residue Y151⁵⁶ in two different RF1 knockout strains (C321.ΔA⁵⁹ and JX33⁶⁹) showed robust decoding activity (η≤75%; FIG. 10a,b). Collectively, these findings agree with prior work that RF1 competition can limit the efficiency of UAGA-qtRNAs³³, and highlight the ability of qtRNA-PACE to rapidly evolve qtRNAs with greatly improved quadruplet codon decoding capabilities in vivo.

Analysis of qtRNA Aminoacylation Fidelity

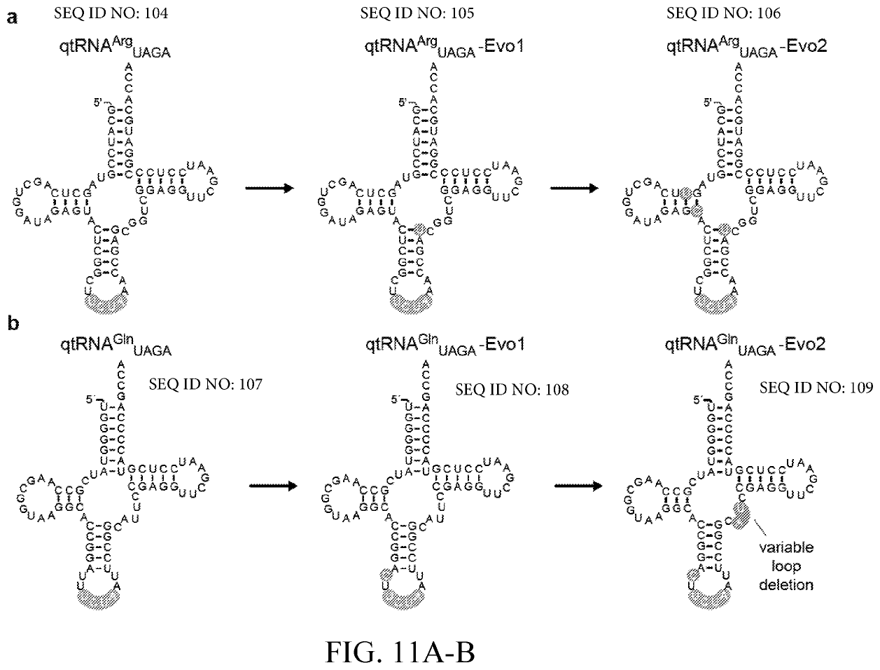

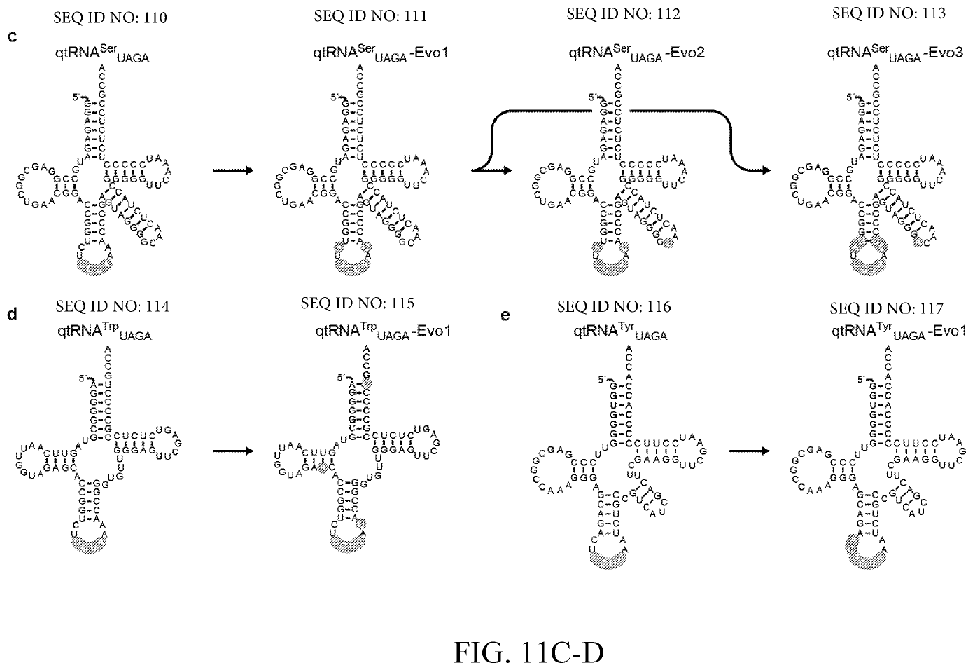

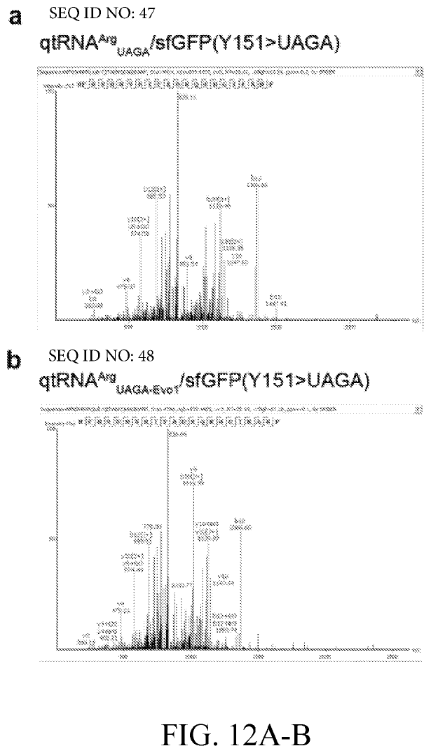

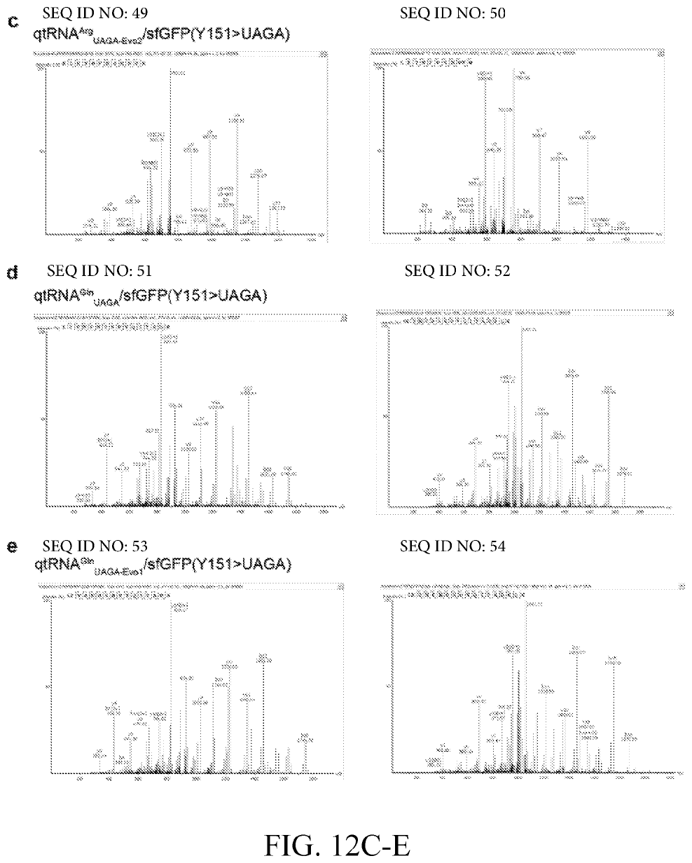

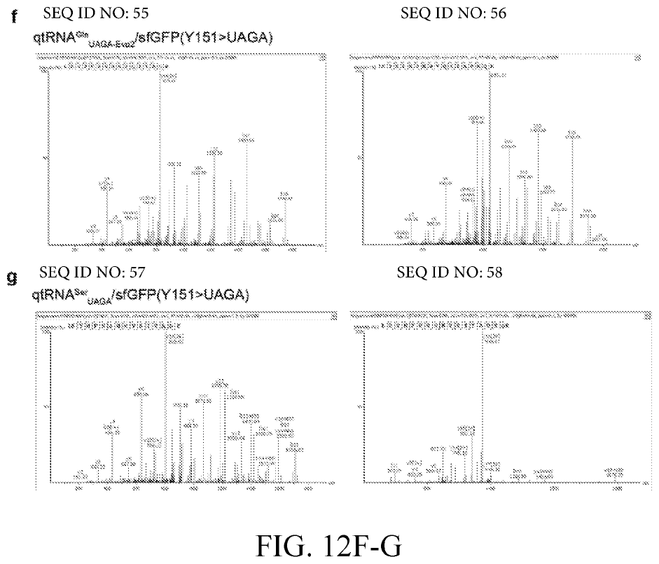

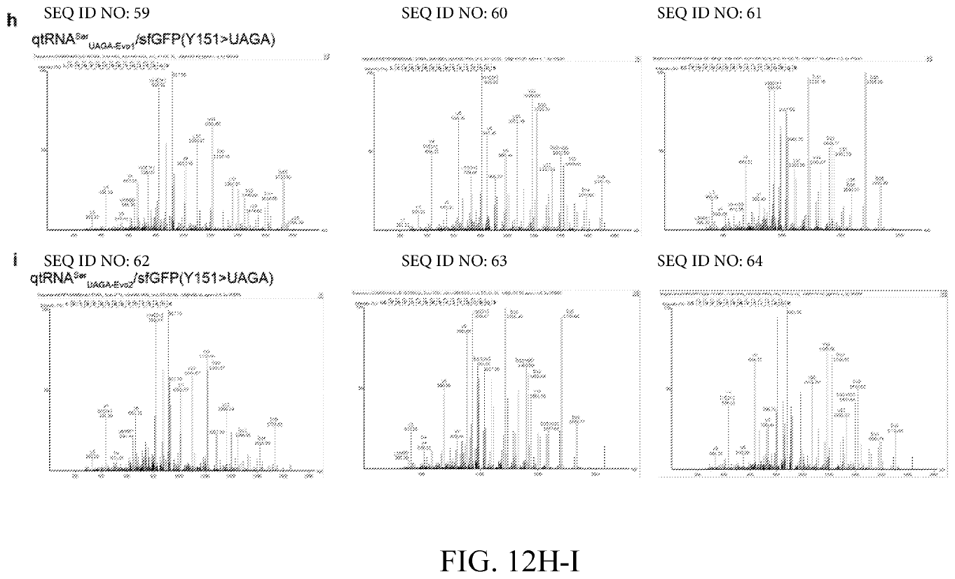

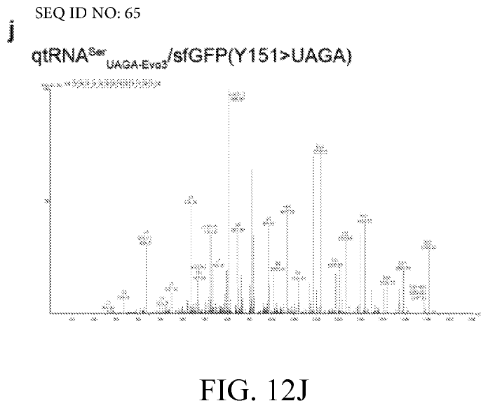

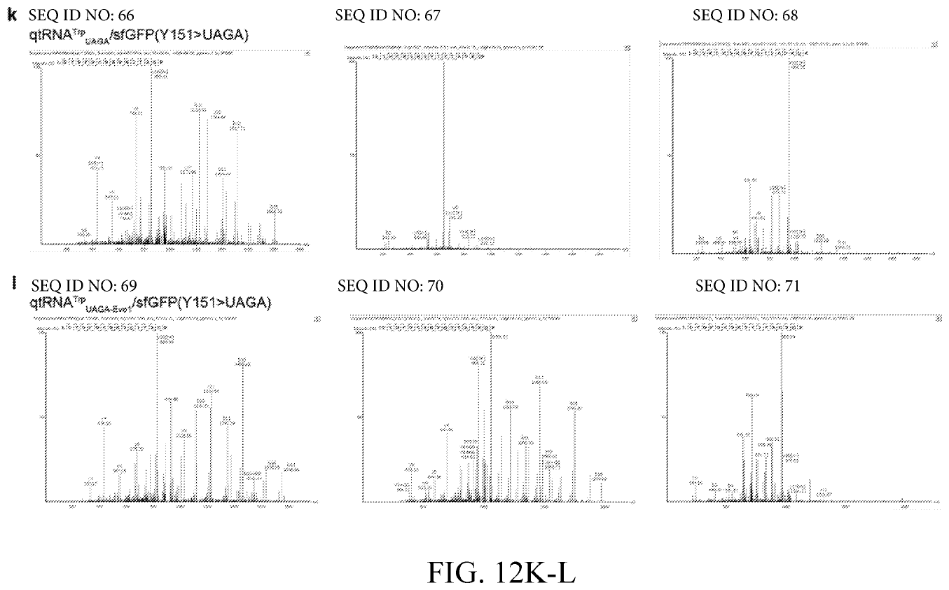

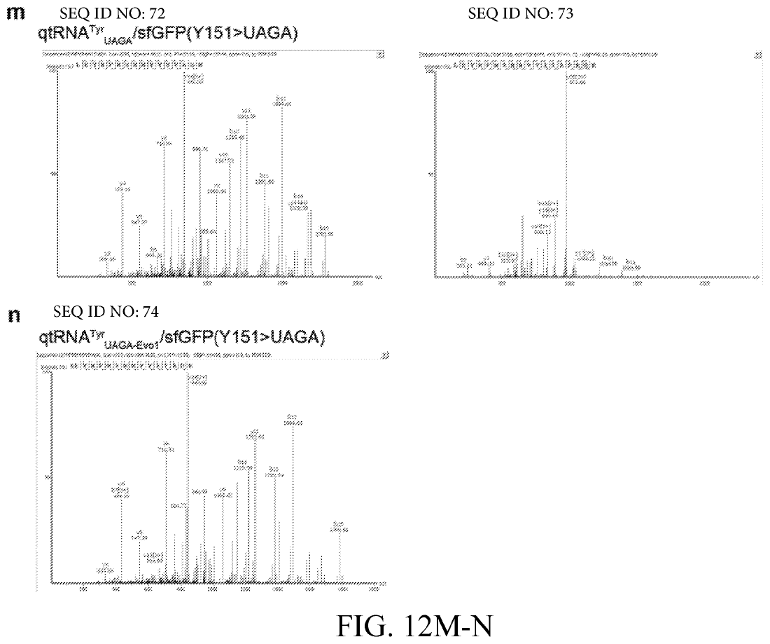

Mutations acquired through qtRNA-PACE campaigns occurred in several regions of the qtRNA (FIG. 11), including interaction interfaces with cognate aminoacyl-tRNA synthetases (aaRSs)⁷⁰. Applicants hypothesized that these mutations may improve quadruplet codon translation by affecting aminoacylation efficiency by the cognate aaRS, or through recruitment of a non-cognate aaRS following adoption of key identity elements. To investigate this possibility, Applicants leveraged the aforementioned sfGFP reporter by incorporating a UAGA codon at residue Y151⁵⁶ and quantified amino acid occupancy via mass spectrometry (FIG. 1f). Applicants find that the expected amino acid was incorporated in both pre- and post-evolution qtRNA^{Gln}_{UAGA}, qtRNA^{Arg}_{UAGA}, qtRNA^{Scr}_{UAGA}, and qtRNA^{Tyr}_{UAGA}variants, with <0.4% contaminating amino acid (Table 4, Supplementary FIG. 12).

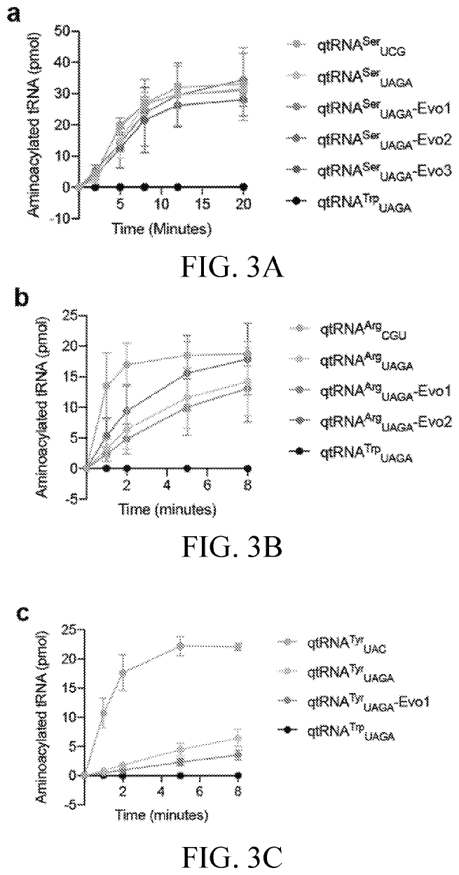

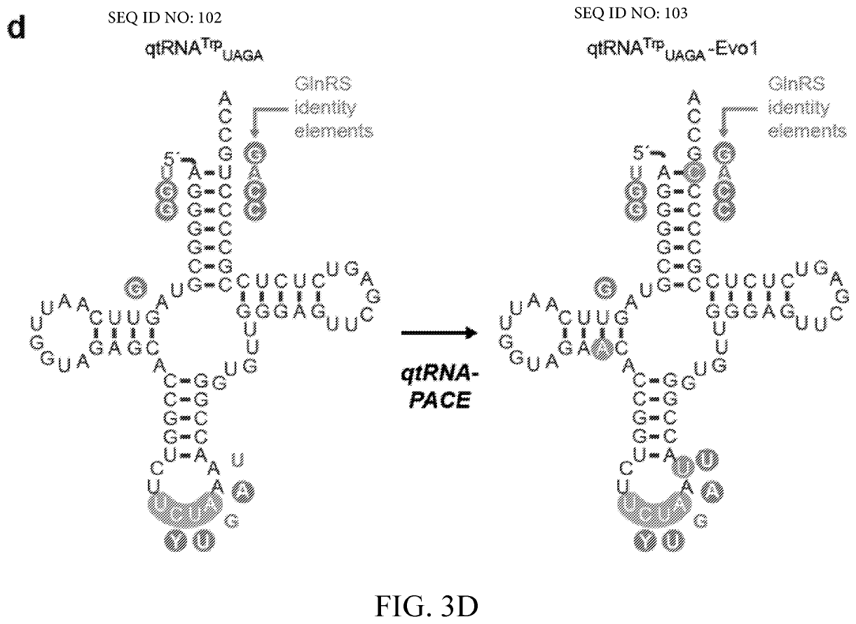

Since the mutations did not affect misacylation by non-cognate aaRSs in vivo, Applicants hypothesized that they may improve the catalytic efficiency of aminoacylation by the cognate aaRS in some cases. Applicants accordingly measured the aminoacylation kinetics of qtRNA^{Arg}_{UAGA}, qtRNA^{Ser}_{UAGA}, and qtRNA^{Tyr}_{UAGA} variants by their cognate E. coli aminoacyl-tRNA synthetases in vitro³⁶ (FIG. 3a-c). Whereas all serine qtRNAs are aminoacylated by EcSerRS with comparable efficiencies, qtRNA^{Arg}_{UAGA} and qtRNA^{Tyr}_{UAGA} variants show moderately abrogated aminacylation kinetics by their cognate aaRSs (EcArgRS and EcTyrRS) as compared to cognate wild-type triplet-decoding tRNAs. As all evolved variants show comparable activities in vivo, these findings may suggest that aminoacylation kinetics are not limiting for quadruplet codon translation in E. coli under the tested conditions.

In contrast, anticodon engineering of qtRNA^{Trp}_{UAGA}resulted in a qtRNA predominantly misacylated with glutamine (81.7% Gln, 5.9% Trp, 12.4% Tyr) (Table 4). The qtRNA-PACE campaign gave rise to three additional base changes: the loss of base pairing at both U12⋅G24 (G24A) in the D-loop and A1⋅U72 (U72C) in the acceptor stem, and A38U in the anticodon loop (FIG. 3d). These changes, in particular A38U which is a known EcGlnRS identity element⁷¹, ensure that qtRNA^{Trp}_{UAGA}-Evo1 is nearly exclusively misacylated with glutamine (99.7% Gln, 0.03% Tyr).

Furthermore, tRNA^{Trp} and tRNA^{Gln} scaffolds are known to switch identity by a C35U substitution in the anticodon⁷². Through examination of the EcGlnRS-tRNA co-crystal structure (PDB: 1GSG⁷³), Applicants noted that tRNA^{Gln}_{CAA} contains the hypermodified nucleotide 5-carboxyaminomethyl-2-thiouridine (cmnm⁵s²U) at anticodon position 34⁷⁴ (FIG. 3e). Given that qtRNA^{Trp}_{UAGA}-Evo1 is an EcGlnRS substrate, it is possible that first two bases of the quadruplet anticodon 5′ UCUA 3′ are accommodated in the space allotted to cmnm⁵s²U34, allowing the third base of the quadruplet anticodon (U) to occupy position 35. These results suggest that qtRNAs will often evolve along trajectories that maintain the aminoacylation profiles of the engineered variants. Future engineering efforts must therefore ensure specific aminoacylation by the cognate synthetase prior to evolution using qtRNA-PACE.

Characterization of Context-Dependence and Processivity of Evolved qtRNAs

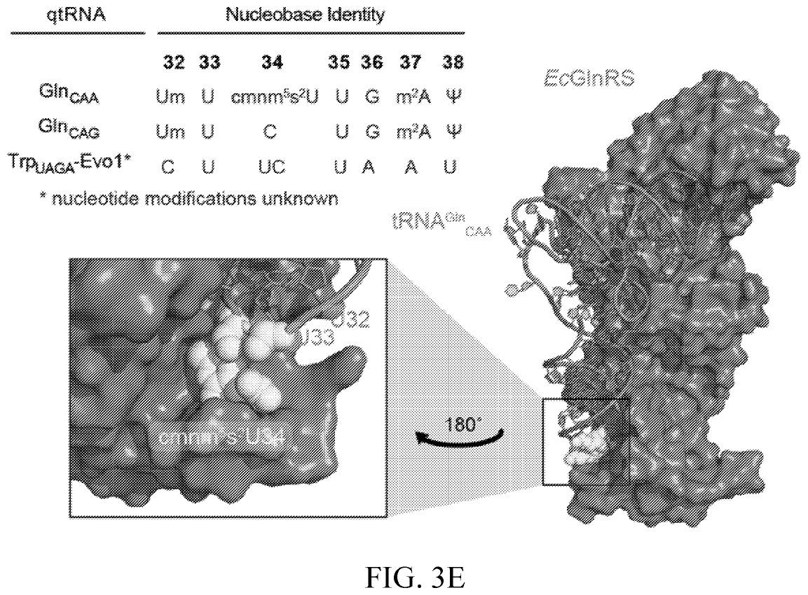

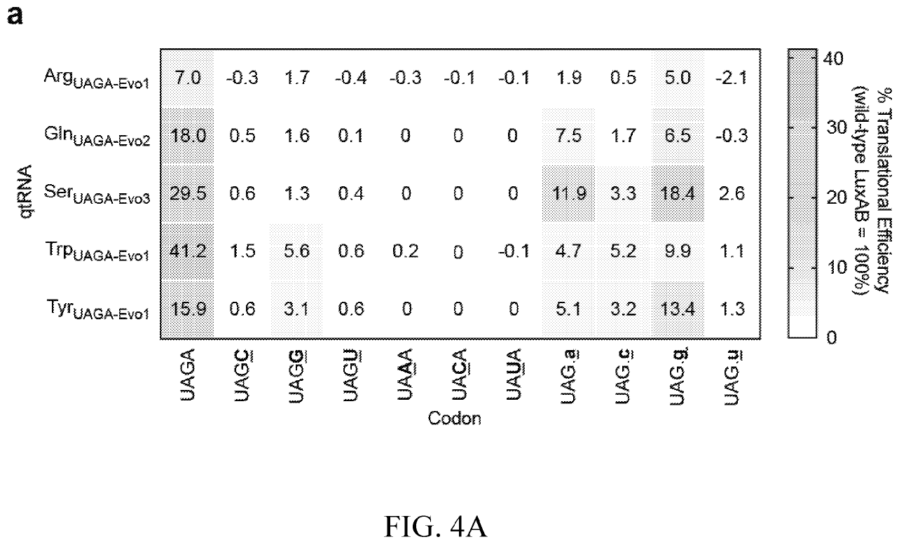

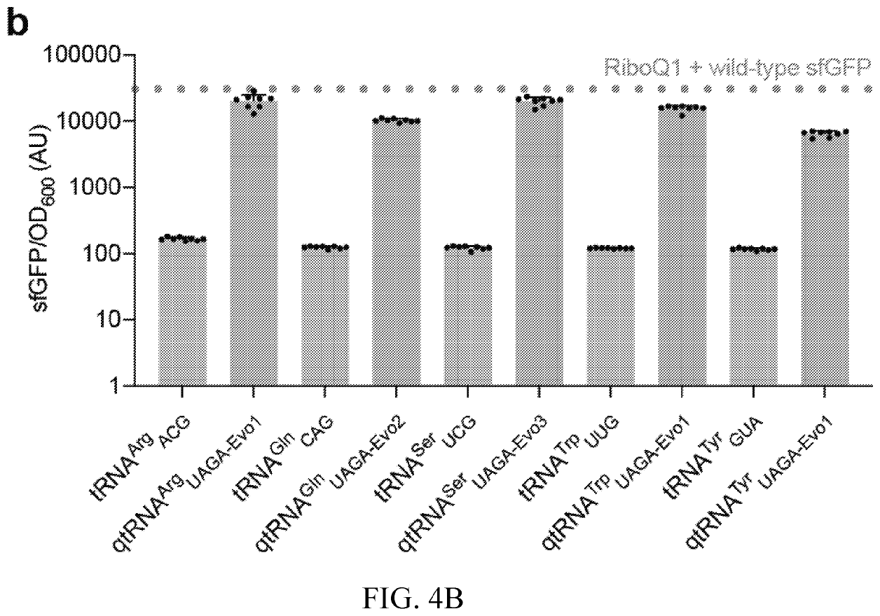

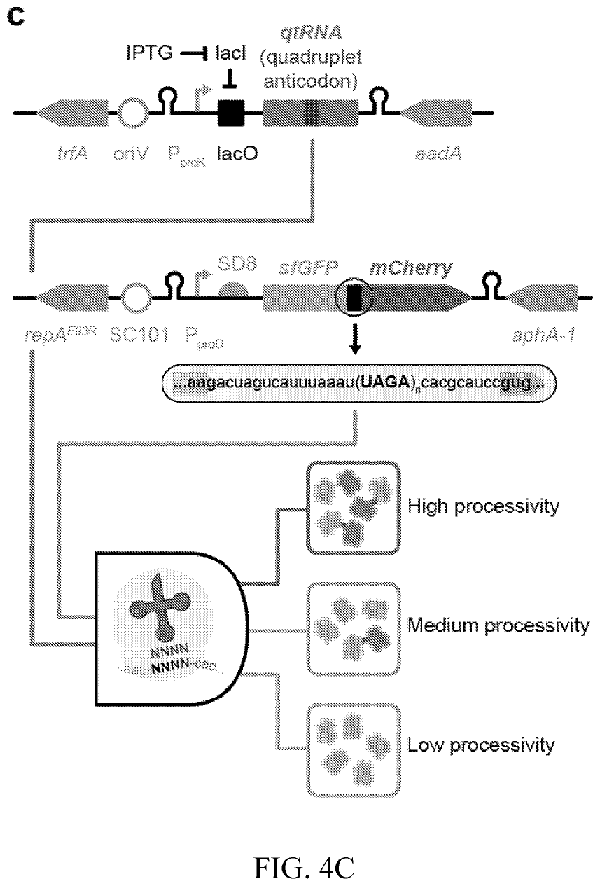

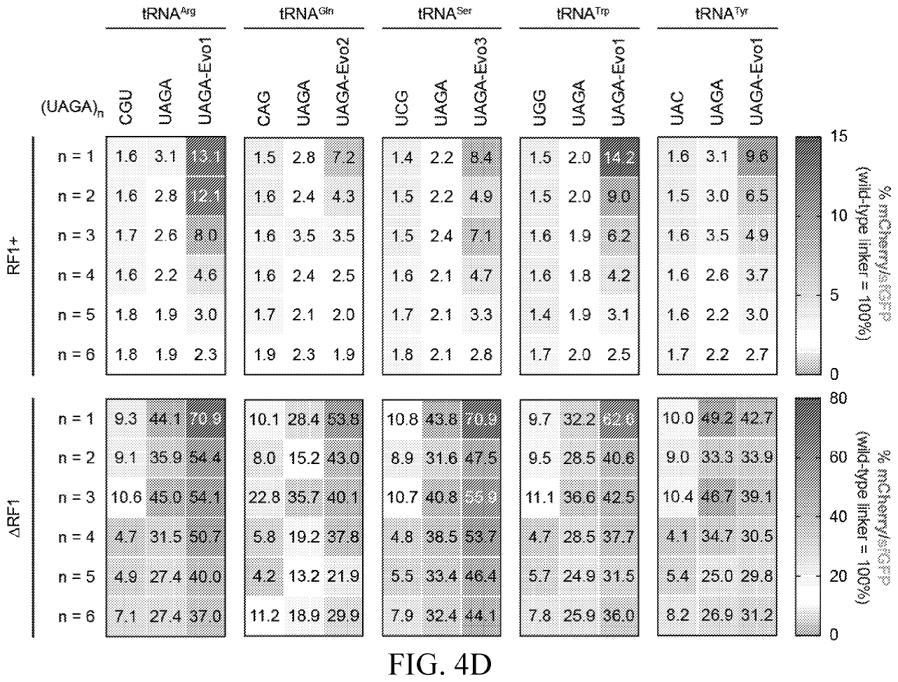

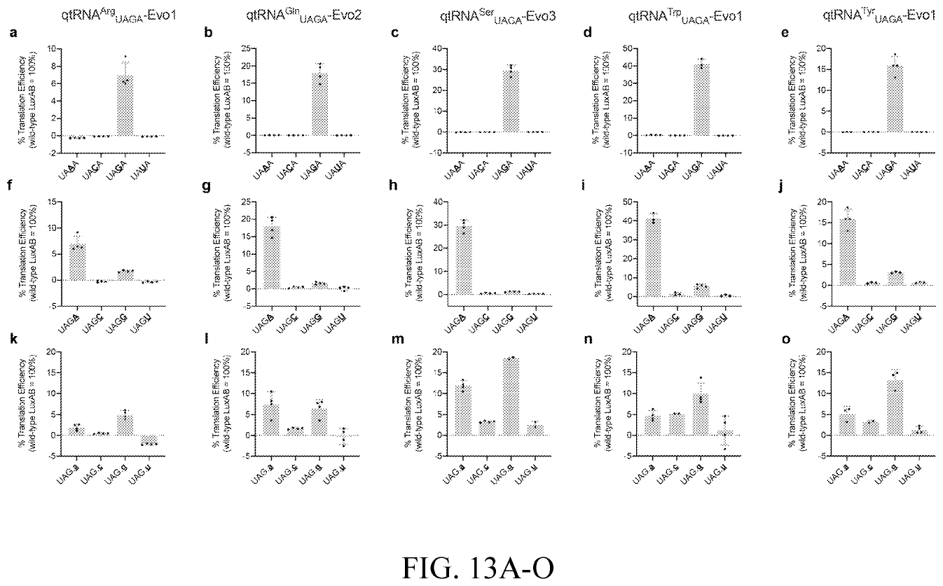

Applicants next explored codon-anticodon crosstalk of PACE-evolved qtRNAs, as canonical tRNAs often decode both cognate and wobble codons. To investigate whether a comparable relationship exists for evolved qtRNAs, Applicants characterized their decoding specificity using the sensitized LuxAB reporter with variations at the third or fourth position of the quadruplet codon (FIG. 4a). Whereas third position variations resulted in ablated luminescence, codons containing a mismatched fourth base were moderately translated, in agreement with prior findings^{23, 26, 75} (FIG. 13). During stringent qtRNA-PACE selections, Applicants noticed the SP genome of qtRNA^{Ser}_{UAGA}evolved UAG codons within the highly expressed phage gene gVIII, suggesting that qtRNAs may crosstalk triplet codons in vivo. Indeed, Applicants find that crosstalk with UAG triplet codons is more prevalent than crosstalk with other near-cognate quadruplet codons (FIG. 4a, FIG. 13). Interestingly, all tested qtRNAs exhibited similar crosstalk trends, suggesting a unifying mechanism to decoding UAGA codons.

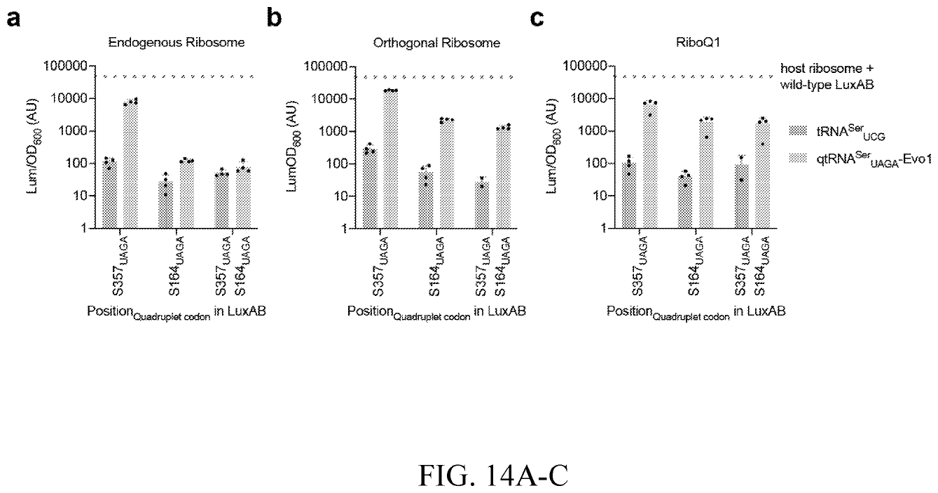

Furthermore, all evolved qtRNAs can decode UAGA codons during translation using an orthogonal ribosome (FIG. 4b). Interestingly, incorporation of rRNA mutations that improve quadruplet codon translation (RiboQ1: U531G/U534A/A1196G/A1197G)¹¹ showed similar efficiency as wild-type host ribosomes (FIG. 14), suggesting that further ribosomal evolution may be necessary to globally enhance quadruplet codon decoding. Applicants hypothesize that future evolution campaigns may integrate an orthogonal ribosome to improve cellular decoding efficiencies

Another factor limiting broad implementation of quadruplet codon translation has been poor processive translation of multiple quadruplet codons, largely due to low qtRNA translation efficiency at single quadruplet codons. To minimize any context dependence and further investigate processivity of quadruplet codon translation, Applicants constructed a dual reporter protein encoding sfGFP and mCherry separated by a linker composed of adjacent UAGA quadruplet codons (FIG. 4c). The efficiency of quadruplet codon translation and processivity can be easily quantified by comparing the relative signal intensities of both fluorescent proteins (FIG. 4d). This strategy has previously been employed to quantify UAG readthrough in eukaryotic cells⁷⁶ and a similar dual fluorescence reporter system has been used to study translational errors and stop codon readthrough in E. coli⁷⁷.

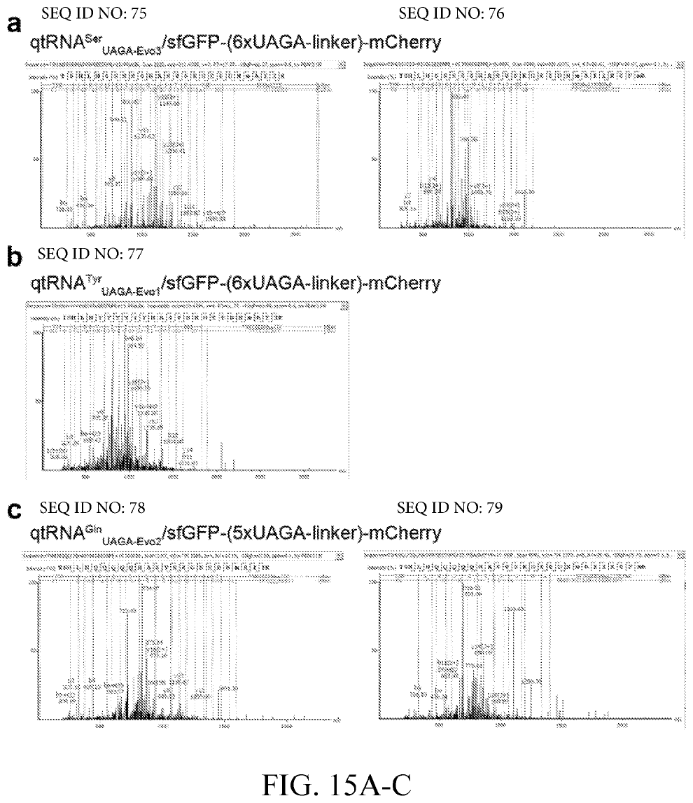

Applicants found that optimized variants qtRNA^{Ser}_{UAGA}-Evo3, qtRNA^{Arg}_{UAGA}-Evo1, qtRNA^{Tyr}_{UAGA}-Evo1, qtRNA^{Trp}_{UAGA}-Evo1, and qtRNA^{Gln}_{UAGA}-Evo2 were able to translate a linker containing up to six adjacent UAGA quadruplet codons (FIG. 4d, FIG. 15). Analysis of the same qtRNA set using the linked sfGFP-mCherry reporters assayed in the JX33 (ΔRF1) strain revealed a greater increase in quadruplet codon decoding processivity, but showed a higher level of readthrough in the absence of the requisite qtRNAs (FIG. 4d). Similar background translation has been noted in ΔRF1 strains in part due to incorporation of triplet codon-encoded canonical amino acids³³.

Suppression of Four Unique, Orthogonal Quadruplet Codons in sfGFP

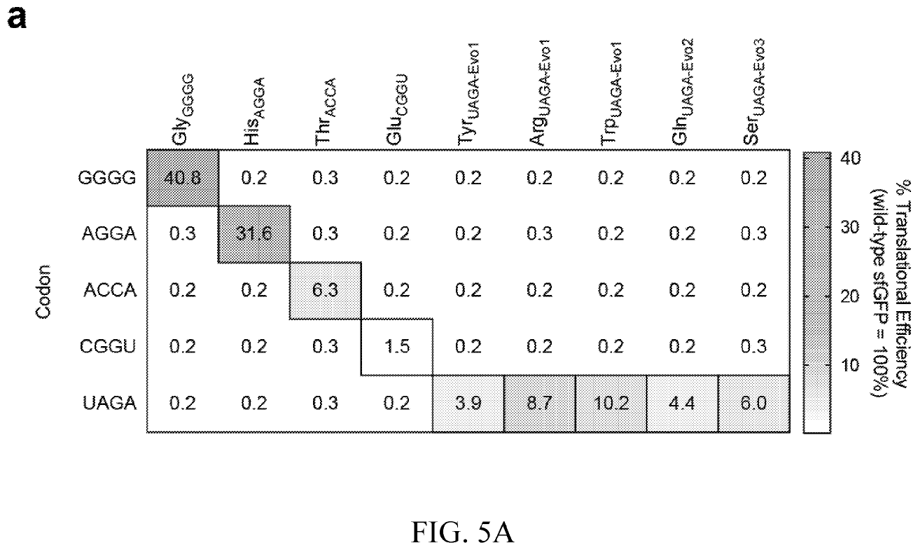

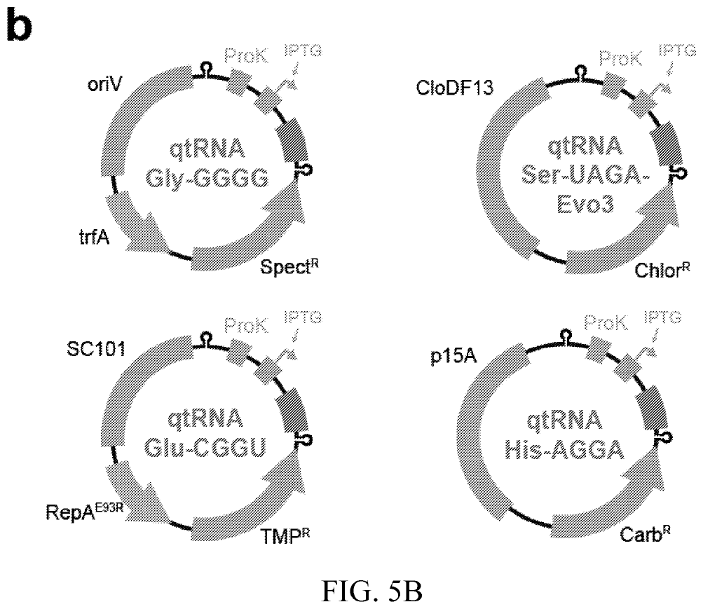

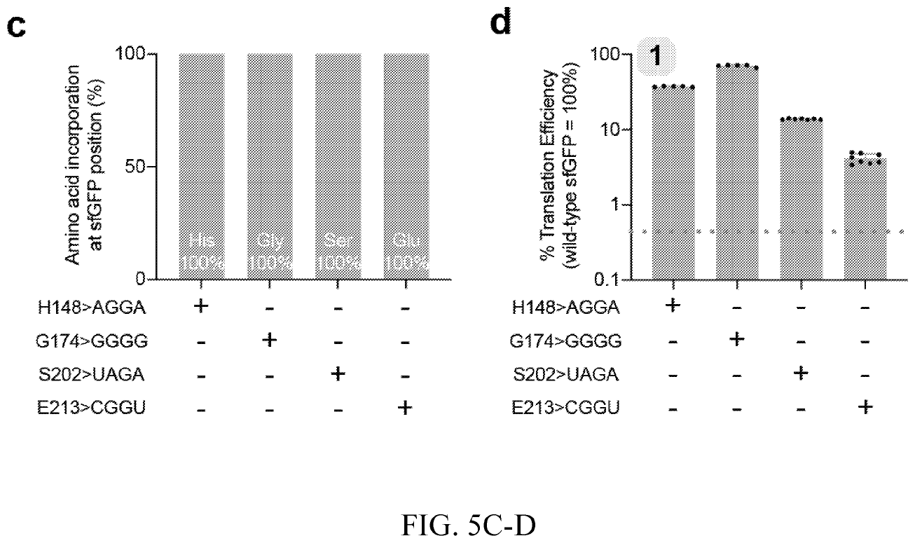

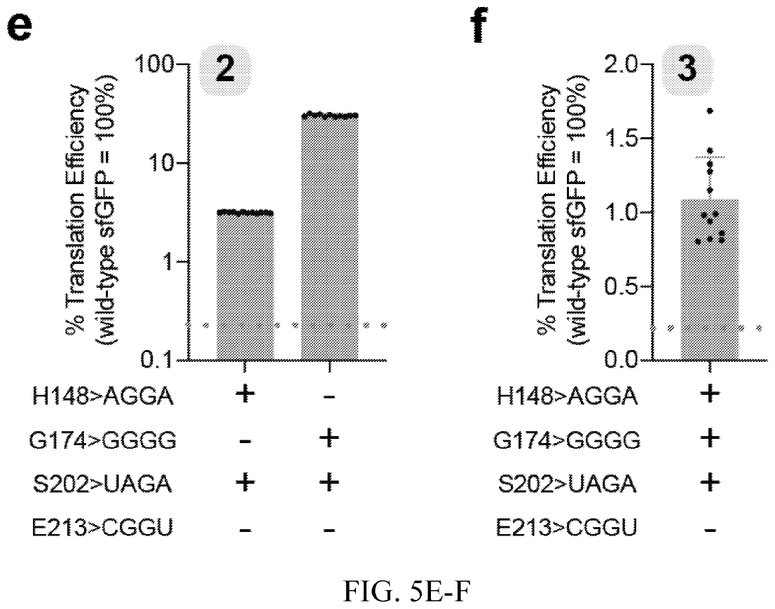

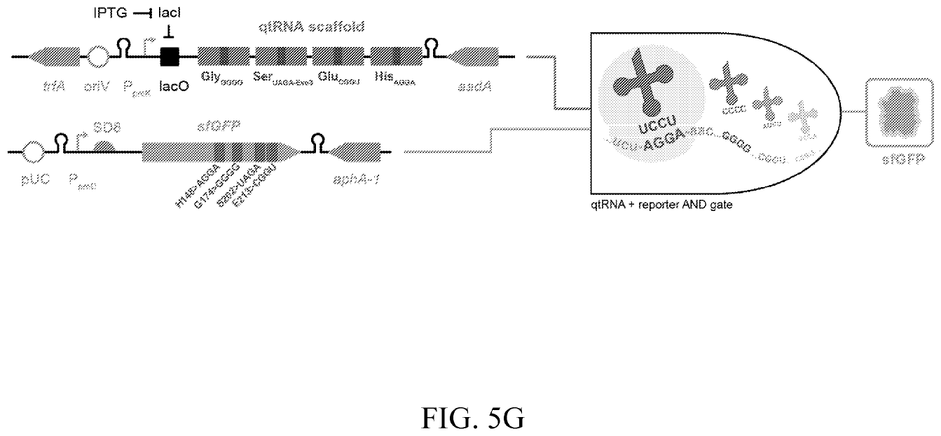

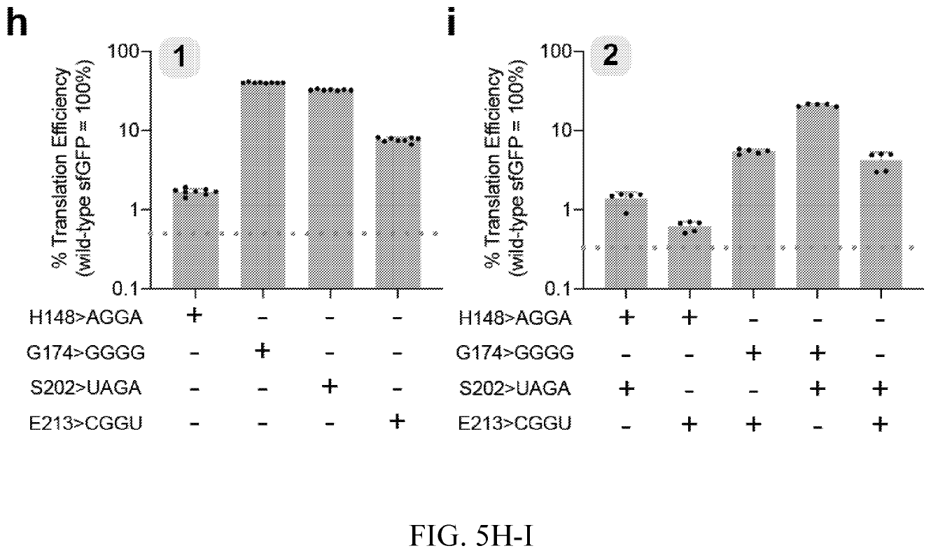

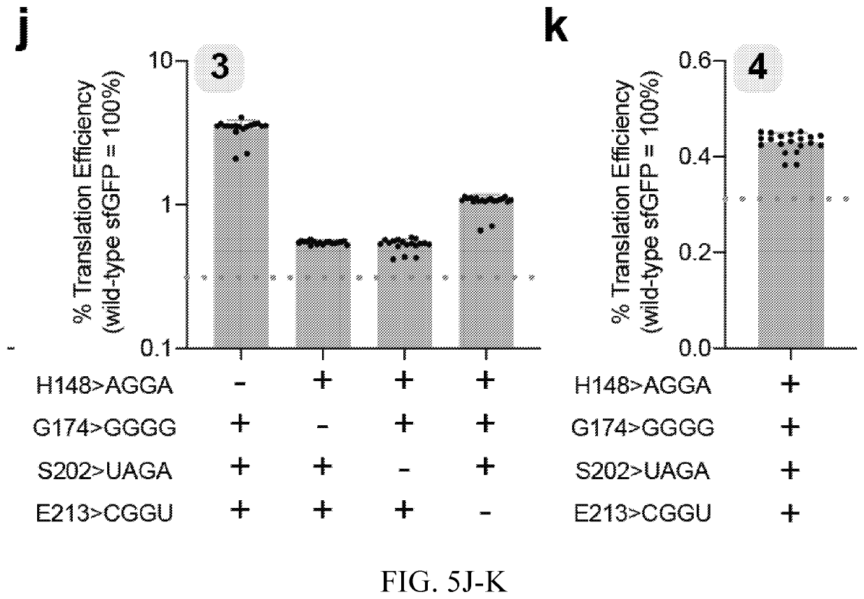

To enable extensive quadruplet codon translation, qtRNAs with efficient and mutually orthogonal decoding capabilities are needed. Applicants therefore leveraged a previously described qtRNA (qtRNA^{Gly}_{GGGG}), variants discovered through lacZ selections (qtRNA^{His}_{AGGA}, qtRNA^{Glu}_{CGGU}), and the most active UAGA-qtRNAs evolved in PACE (qtRNA^{Tyr}_{UAGA}-Evo1, qtRNA^{Arg}_{UAGA}-Evo1, qtRNA^{Trp}_{UAGA}-Evo1, qtRNA^{Gln}_{UAGA}-Evo2, and qtRNA^{Ser}_{UAGA}-Evo3) to investigate their mutual orthogonality. Using sfGFP with the corresponding quadruplet codons at position Y151, Applicants find exceptional degrees of orthogonality between all tested qtRNAs (FIG. 5a).

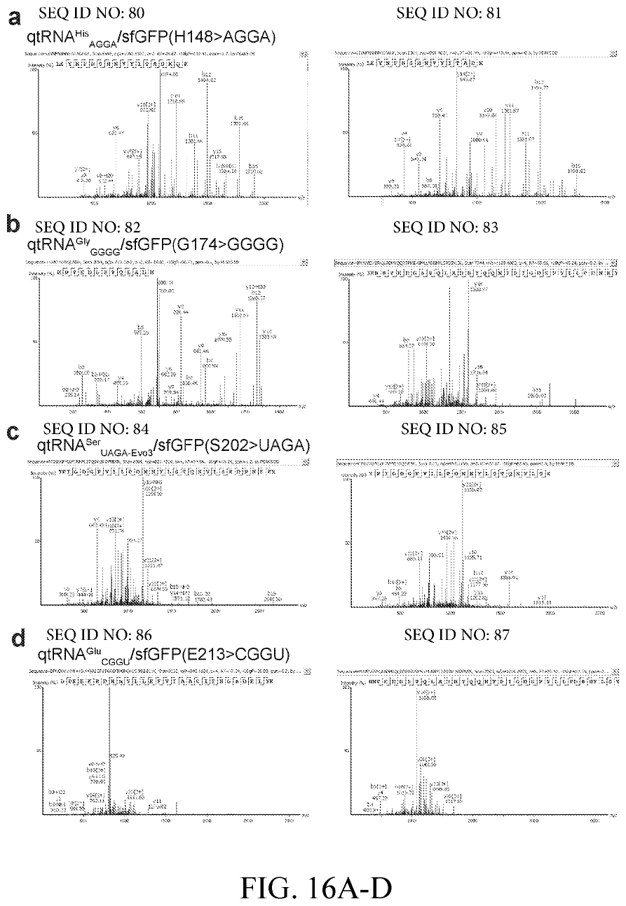

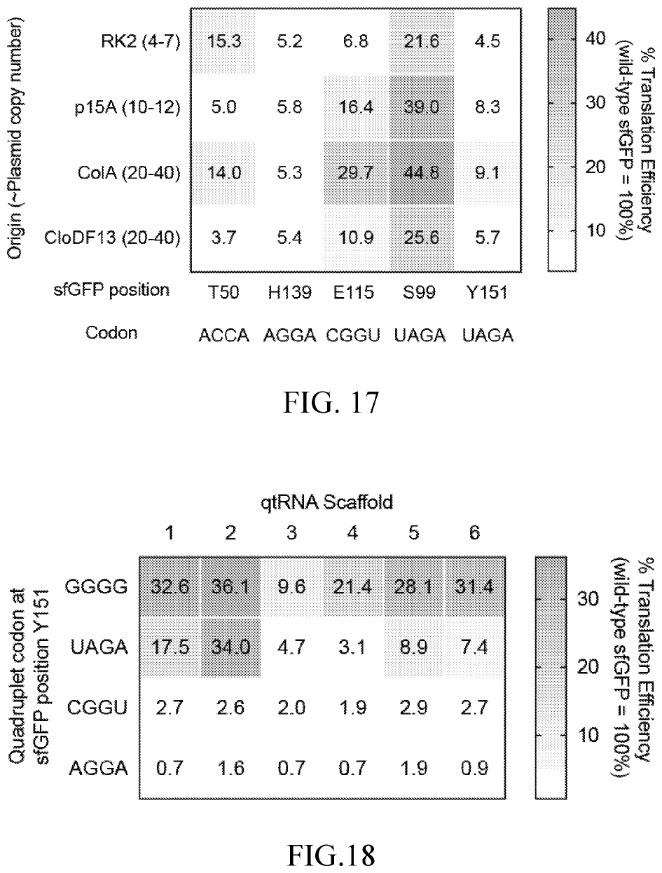

Emboldened by these findings, Applicants explored the ability of four qtRNAs to function alongside one another and translate sfGFP through multiplexed decoding of cognate quadruplet codons. To date, only two mutually orthogonal qRNAs have been reported to work in concert in living cells and decode their cognate quadruplet codons^{34, 78}. To permit the exploration of >2 decoding events in a single reporter, Applicants developed a series of qtRNA expression plasmids encoding mutually orthogonal origins of replication and resistance markers, enabling their concurrent use in a single E. coli cell (FIG. 5b). For all assays, Applicants used the most active UAGA-qtRNAs based on faithful amino acid incorporation (Table 2). Applicants first confirmed exclusive incorporation of the expected amino acids at various positions throughout sfGFP (FIG. 5c, FIG. 16). Using these orthogonal plasmids, Applicants were able to translate one (η−4-37%; FIG. 5d), two (η−3-30%; FIG. 5e), and three (η−1%; FIG. 5f) unique quadruplet codons in sfGFP. On average, trends in the observed qtRNA decoding efficiencies aligned moderately with plasmid copy number (FIG. 17), suggesting that they may be constrained by qtRNA abundance upon induction as previously noted⁷⁹.

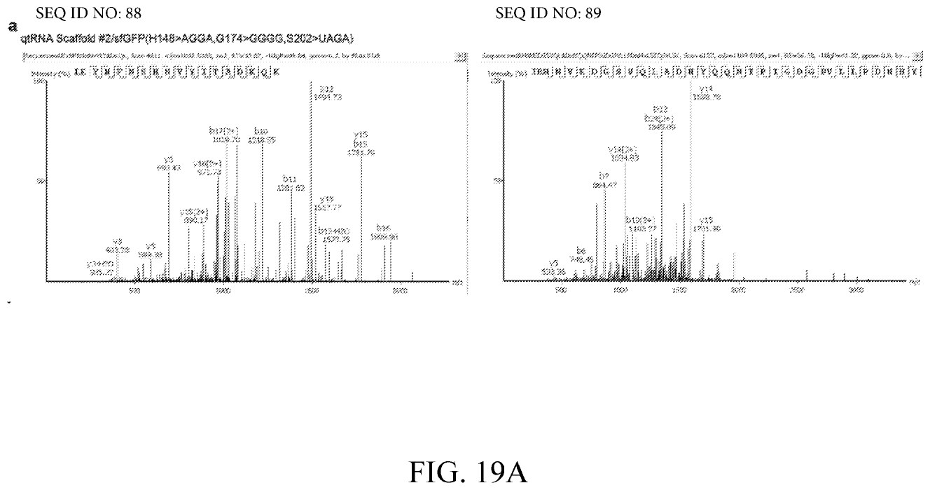

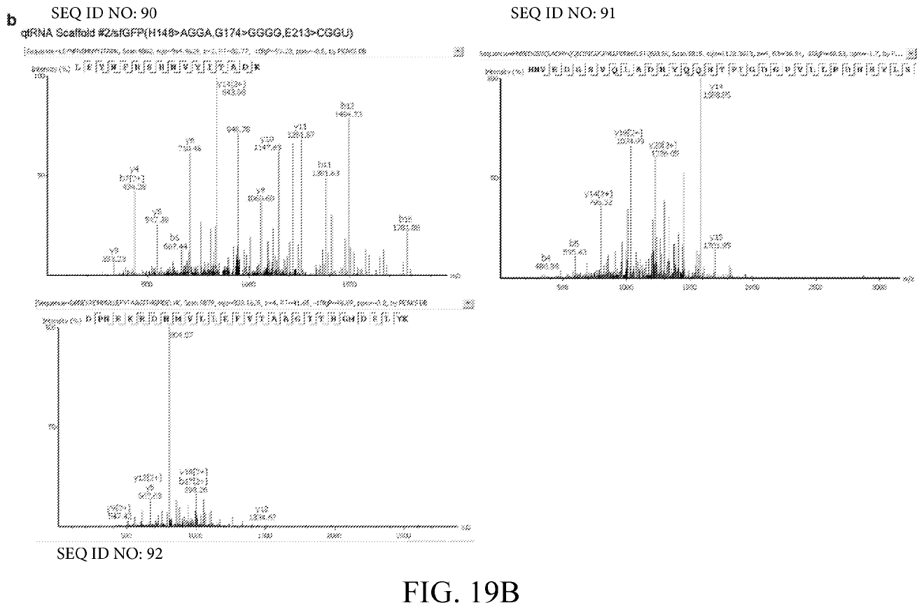

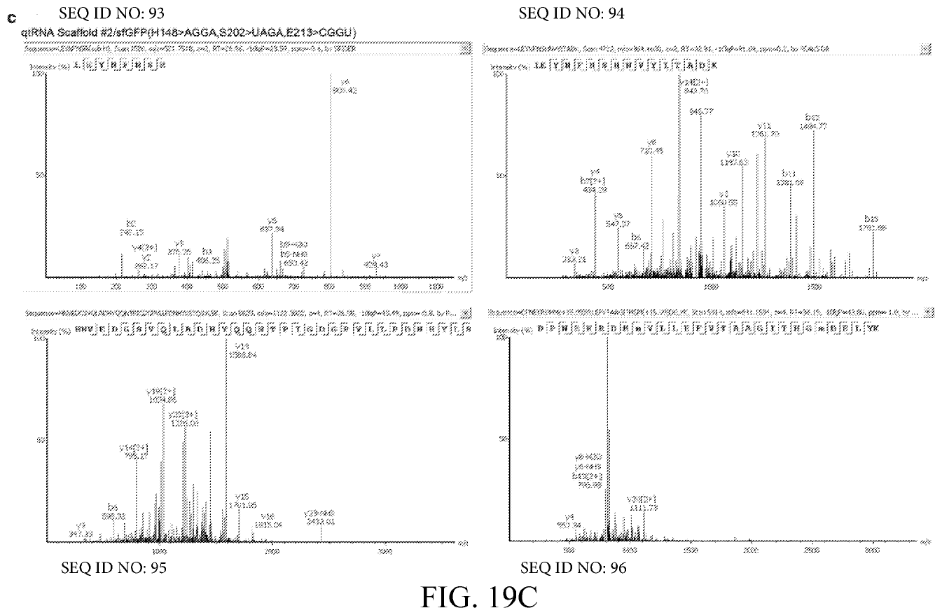

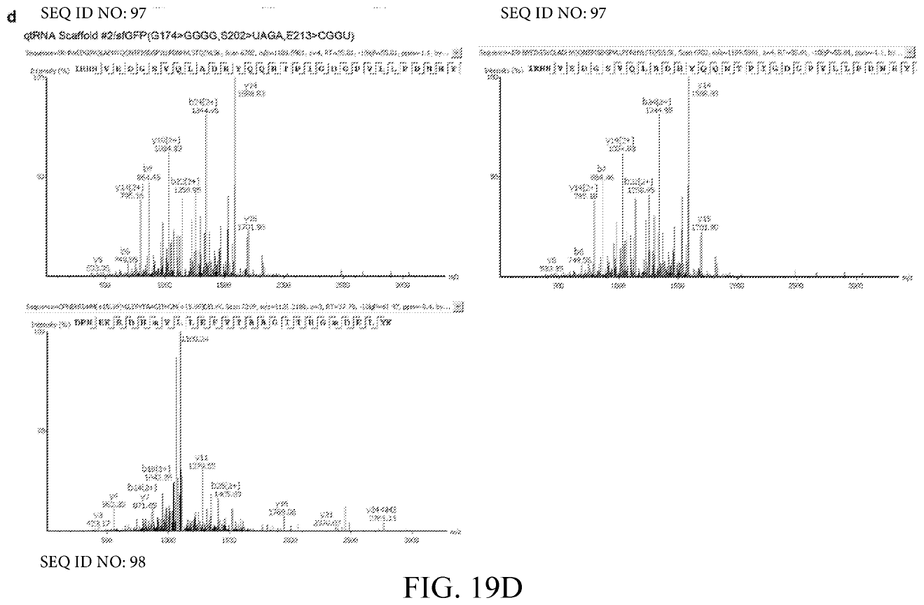

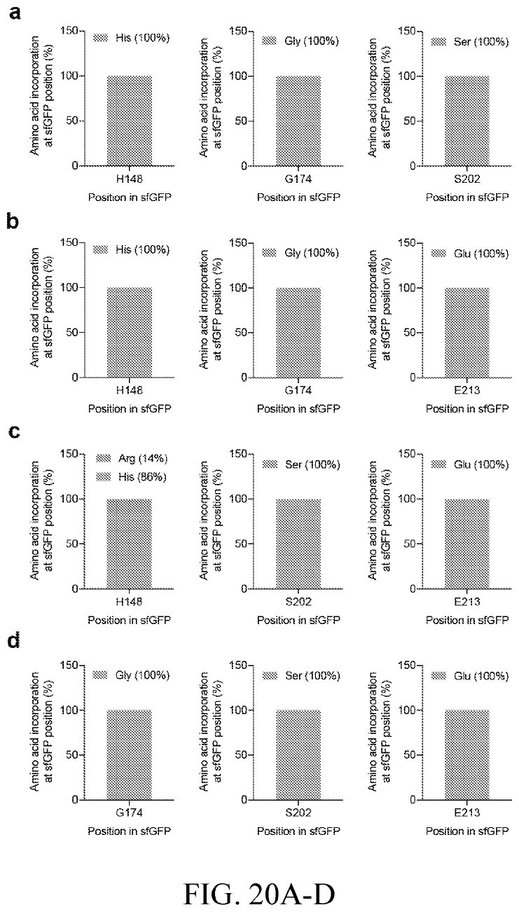

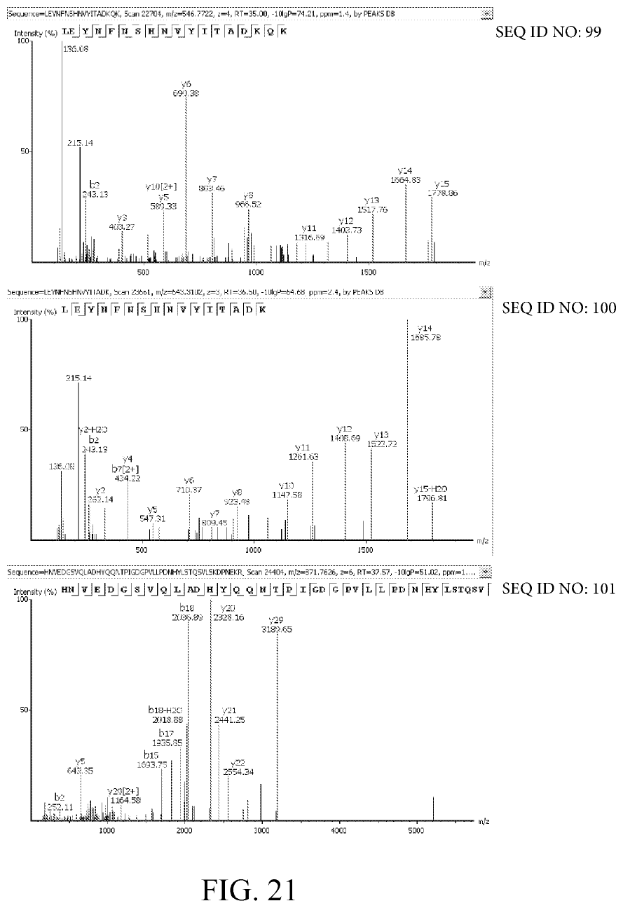

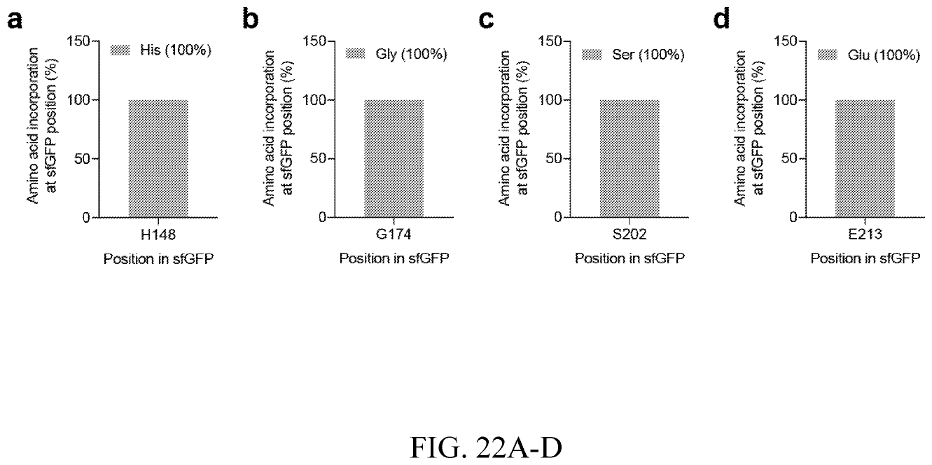

Attempts to extend these results to four unique qtRNA expression plasmids showed fitness defects in the resultant strains, likely due to plasmid load on the cell (Table 5). To minimize the number of orthogonal plasmids necessary to decode more than three events in a single reporter, Applicants hypothesized that Applicants could create single plasmids capable of expressing multiple unique qtRNAs via multicistronic cassettes. To obviate the creation of highly repetitive synthetic qtRNA operons, Applicants instead mined endogenous multicistronic tRNA operons from E. coli and chose six unique scaffolds that contain at least four tRNAs each as the basis for the synthetic constructs (Table 6). Guided by qtRNA decoding efficiency in LuxAB and sfGFP assays, Applicants repurposed these six scaffolds to include qtRNAs in descending order of activity: qtRNA^{Gln}_{GGGG}, qtRNA^{Ser}_{UAGA}-Evo3, qtRNA^{Glu}_{CGGU}, then qtRNA^{His}_{AGGA} (FIG. 5g, Table 7). Of the six qtRNA scaffold assayed, scaffold #2 showed the highest overall percent translational efficiency (FIG. 18). Further interrogation of qtRNA scaffold #2 showed efficient decoding of one (η=1.5-40%; FIG. 5h), two (η=0.6-22%; FIG. 5i), and three (n=0.5-4%; FIG. 5j) quadruplet codons in sfGFP. Excitingly, Applicants find that qtRNA scaffold #2 also enables translation of four unique quadruplet codons (η=0.45%) in sfGFP (H148>AGGA, G174>GGGG, S202>UAGA, and E213>CGGU) in a qtRNA-dependent manner (FIG. 5k). Specific amino acid incorporation corresponding to translation of three (FIGS. 19, 20) and four quadruplet codons (FIGS. 21, 22) in sfGFP were validated via protein purification and mass spectrometry. Taken together, these results demonstrate the first successful decoding of four unique quadruplet codons in living cells.

### Discussion

Genetic code expansion methods have historically been limited by low efficiency^{11, 32-34}, competition with host cellular factors²⁸⁻³⁰, and the need for whole genome codon reassignment¹⁻⁵. To circumvent these issues, Applicants first developed a selection scheme using E. coli β-galactosidase to nominate functional and amino acid-specific codon-anticodon pairs that could serve as robust starting points for further optimization (FIG. 1a). To improve quadruplet codon-decoding capabilities of qtRNAs corresponding to canonical amino acids, Applicants developed a system for the unbiased, phage-assisted continuous evolution of qtRNA scaffolds beyond the anticodon and flaking sequences (FIG. 2b). By evolving five unique UAGA-decoding qtRNAs, Applicants noted a general trend wherein mutations immediately flanking the anticodon were acquired and greatly improved their activities (FIG. 2f-g, Table 4). Using these nominated qtRNAs, Applicants sought to further investigate context-dependence and processivity (FIG. 3-4). Finally, towards the extensible development of an in vivo quadruplet codon-based translation system, Applicants utilized multicistronic tRNA operons allowing four qtRNAs to function in concert to simultaneously decode up to four mutually orthogonal quadruplet codon decoding events in a single reporter for the first time (FIG. 5, Table 6).

The development of a plasmid-based system that capitalizes on endogenous E. coli translational machinery, confers minimal host fitness defects, and does not require extensive strain engineering overcomes major limitations of current genetic code expansion technologies. Using the newly discovered and evolved qtRNAs, Applicants explored key properties essential for a quadruplet codon-based genetic code, including characterization of quadruplet codon specificity and crosstalk, and UAGA-decoding qtRNA competition with RF1 (FIG. 3,4d). Applicants showed that qtRNAs crosstalk with quadruplet codons that mismatch at the fourth position, quantified a previously unidentified form of crosstalk with related triplet codons (FIG. 4a), and illustrated the efficient use of a previously described orthogonal ribosome to enable quadruplet-codon translation (FIG. 4b). Investigating qtRNA competition with RF1 wherein engineered UAGA-qtRNAs repurpose the low-usage UAG stop codon, Applicants illustrate increased qtRNA suppression activity in a RF1 knockout strain (FIG. 4d). This not only showed effective competition with RF1, but also that evolved qtRNAs have strain-independent suppression improvements. Furthermore, Applicants demonstrated processive protein translation using a non-canonical genetic code for the first time, a corollary of the high efficiency of the qtRNAs and an essential feature for future quadruplet codon decoding (FIG. 5). Notably, qtRNA-PACE-derived mutations in the anticodon flanking sequences that increased suppression activity may suggest the existence of general patterns that govern quadruplet anticodon efficiency⁸⁰, as have been previously described for triplet codons⁶³, and providing a systematic route to improve qtRNA activities.

Cumulatively, the findings highlight significant qtRNA evolvability for efficient and amino acid-specific quadruplet codon translation, demonstrating essential properties necessary for the development of an exclusively quadruplet codon code. Through the extension of this directed evolutionary framework and the newly developed orthogonal multi-qtRNA expression plasmids, rRNA evolution strategies may be necessary to eliminate crosstalk with triplet codons and maintain quadruplet coding frames. In particular, targeted engineering of ribosomal conformational may further improve decoding fidelity beyond the recently described +1 frameshifting during tRNA-mRNA translocation²². Additionally, Applicants expect that the newly designed multicistronic qtRNA cassettes, alongside methods for ncAA incorporation and negative selection during continuous evolution³⁵, will allow for the simultaneous directed evolution of dedicated qtRNA-synthetase pairs for canonical and/or non-canonical amino acids. This work therefore bolsters the toolkit available to synthetic biologists investigating genetic code expansion in vivo, and may serve as the basis for the creation of de novo quadruplet codon/qtRNA pairs for each of the 20 canonical amino acids.

### Methods

General methods. Antibiotics (Gold Biotechnology) were used at the following working concentrations unless otherwise noted: carbenicillin, 50 μg/mL; spectinomycin, 100 μg/mL; chloramphenicol, 40 μg/mL; kanamycin, 30 μg/mL; tetracycline, 10 μg/mL; streptomycin, 50 μg/mL. David Rich Medium (DRM)²⁰ was used for PACE and all experiments involving plate reader measurements, excluding experiments involving JX33 RF1 knockout strain. For all other purposes, including phage-based selection assays, general cloning, and all experiments involving JX33 RF1 knockout strain, 2× YT media was used. All PCRs were performed using Phusion U HotStart DNA Polymerase (Life Technologies). Key plasmids from this study have been deposited on Addgene. See the Extended Supplement for all plasmids and plasmid maps, Addgene links, catalog numbers of materials, and plasmids used to produce each figure.

Chemically competent cell preparation. Strain S3489, a K12 derivative of S2060⁸² further optimized for directed evolution by deletion of ribosome hibernation-promoting factor Hpf (Liu et al, submitted and will be reported elsewhere), was used in all reporter assays, phage propagation assays, plaque assays, and PACE campaigns unless otherwise noted. To prepare competent cells, an overnight culture was diluted 1,000-fold into 50 mL of 2× YT media supplemented with maintenance antibiotics and grown at 37° C. with shaking at 230 rpm to OD₆₀₀ ˜0.4-0.6. Cells were pelleted by centrifugation at 6,000× RCF for 10 min at 4° C. The cell pellet was then resuspended by gentle stirring in 5 mL of TSS (LB media supplemented with 5% v/v DMSO, 10% w/v PEG 3350, and 20 mM MgCl₂). The cell suspension was stirred to mix completely, aliquoted, and flash-frozen in liquid nitrogen, and stored at −80° C. until use.

USER cloning. Plasmids were cloned using USER (Uracil-Specific Excision Reagent) assembly, wherein primers were designed to include a USER junction, denoting the region between the 5′ primer end containing a dA and a deoxyuracil base approximately 15 base pairs downstream. USER junctions were additionally designed to have a 55° C.<Tₘ<60° C. and minimal secondary structures. PCR products were run on a 1% agarose gel containing approximately 0.2 μg/mL ethidium bromide, allowing visualization under ultraviolet light, and subsequently purified using QIAquick Gel Extraction kit (Qiagen). Fragments were quantified using a NanoDrop 1000 Spectrophotometer (Thermo Fisher Scientific). Fragments containing complementary USER junctions were added in an equimolar ratio of between 0.2-1 pmol to a 10 μl reaction containing 1 μl CutSmart Buffer (50 mM potassium acetate, 20 mM Tris-acetate, 10 mM magnesium acetate, 100 μg/mL BSA at pH 7.9; New England Biolabs), 0.75 μL DpnI (New England Biolabs), and 0.75 μL USER enzyme (Uracil-DNA Glycosylase and DNA-glycosylase-lyase Endonuclease VIII; New England Biolabs). Reactions were incubated at 37° C. for 20 min, then heated to 80° C. for 3 min, and slowly cooled to 12° C. at 0.1° C./s. During this assembly, uracil DNA-glycosylase catalyze the excision of a dU, creating an apyrimidinic site at which Endonuclease VIII breaks the phosphodiester backbone. Assembled constructs were added to 100 μL, 2× KCM (100 mM KCl, 30 mM CaCl₂, 50 mM MgCl₂ in MilliQ H₂O) and 100 μL competent cells. For all cloning, Applicants used either Mach1F (Mach1 T1^{R} cells (Thermo Fisher Scientific) mated with F′ episome of S2060 strain³¹), NEB Turbo (New England Biolabs), DH5α (Thermo Fisher Scientific), or 10-beta (New England Biolabs) cells. Cells were flicked to mix and incubated on ice for 10 min, heat shocked at 42° C. for 1.5 min, and then placed back on ice for 2 min. Cells were allowed to recover in 1 mL 2× YT at 37° C. with shaking between 230-300 RPM for at least 45 min. Cells were then streaked on 1.5% agar-2× YT supplemented with appropriate antibiotics and incubated at 37° C. for 16-18 hours.

Transformation of chemically competent cells. To transform cells, 100 μL of competent cells were thawed on ice. To this, plasmid (2 μL each of miniprep-quality plasmid; up to two plasmids per transformation) and 100 μL KCM solution (100 mM KCl, 30 mM CaCl₂, and 50 mM MgCl₂ in H₂O) were added and flicked to mix. For transformations of greater than two plasmids, 2 μL of each plasmid was added to 30 μL competent cell/KCM mix. The mixture was incubated on ice for 10 min and heat shocked at 42° C. for 90 s. The mixture was chilled on ice for 4 min, then 1 mL of 2× YT media was added. Cells were allowed to recover at 37° C. with shaking at 230 rpm for at least 45 min, streaked on 2× YT media+1.5% agar plates containing the appropriate antibiotics, and incubated at 37° C. for 16-18 h.

Selection criteria for representative E. coli tRNAs scaffolds. In cases where quadruplet-decoding tRNAs had been previously engineered, evolved, or discovered as natural suppressors, the same tRNA scaffolds were used in the evolution studies. This criterion was met for four tRNAs (Gly, Leu, Ser, Thr). In cases where either only a single copy of the tRNA scaffold was present in the E. coli genome (Cys, Trp) or all copies are genotypically identical with exception of the anticodon (Asn, Asp, Glu, His, Ile, Lys, fMet, Phe, Tyr), the choice was constrained to a single genotype. This criterion was met for eleven tRNAs. In all other cases, Applicants chose tRNA scaffolds that were found at the end of their respective operons⁸³ or sufficiently separated from neighboring tRNAs in order to use tRNAs that are endogenously expressed at high levels, ensure that Applicants would not inadvertently capture >1 tRNA during amplification and cloning into the tRNA expression plasmid, and limit amplification issues using primers that may anneal within the highly structured sequence of a neighboring tRNA. This criterion was met for the remaining six tRNA (Ala, Met, Pro, Gln, Arg, Val). tRNA genes were amplified directly from E. coli genomic DNA.

LacZ selections. To nominate positions in lacZ for amino-acid specific selections, Applicants first confirmed the dependence of key positions in lacZ on the identity of the incorporated amino acid. Oligos bearing degenerate triplet codons (NNN) at requisite positions in lacZ were used to generate libraries of the constitutive lacZ-expressing plasmids pAB191b using USER cloning. Chemically competent BW25113 ΔlacI (Strain JW0336-1; CGSC #: 8528) cells were transformed with the libraries, recovered for 2 hours at 37° C. with shaking at 230 rpm, then plated as a serial dilution series on M9 minimal medium plates with glucose 1.8% agar, 0.01% thiamine, 22 mM glucose, 40 μg/mL chloramphenicol) or lactose (1.8% agar, 0.01% thiamine, 11 mM lactose, 0.033% Bluo-Gal, 40 μg/mL chloramphenicol) and incubated at 37° C. for 24-72. Libraries all showed >100-fold coverage as gauged by transformation efficiency (>6400 total CFUs), and comparison of the total (glucose) to LacZ+ (lactose) transformants was used to inform amino acid dependence. For positions where the observed frequency agreed with the expected frequency of LacZ+ colonies, 16-32 unique colonies were picked and surveyed by Sanger sequencing at the randomized residue. Positions which showed triplet codons exclusive to a single amino acid were used for quadruplet codon-qtRNA coevolution studies. For these library-cross-library selections, the identical protocol was used to generate the lacZ codon libraries with the exception that the used oligos encoded fully degenerate quadruplet codons (NNNN) at the requisite positions. Following plating on glucose, transformed colonies were used to make competent cells, which were later transformed with qtRNA libraries bearing fully degenerate anticodons (NNNN). Co-transformation efficiencies corresponded to >100-fold in all cases (>6.6E6 total CFUs). Single clone sequencing at the codon (lacZ) and anticodon (qtRNA) showed the identical sequences in most cases for colonies picked from lactose plates.

Measurement of quadruplet codon translation efficiency using luciferase reporter. S3489 cells were transformed with the luciferase-based activity reporter and qtRNA expression plasmids. Overnight cultures of single colonies grown in DRM media supplemented with maintenance antibiotics were diluted 500-fold into DRM media with maintenance antibiotics in a 96-well 2 mL deep well plate, with or without IPTG. The plate was sealed with a porous sealing film and grown at 37° C. with shaking at 900 RPM. After 1 hour, 175 μL of cells were transferred to a 96-well black-walled clear-bottom plate, and then 600 nm absorbance and luminescence were read using a ClarioSTAR plate reader (BMG Labtech) over the course of 8 h, during which the cultures were incubated at 37° C. IPTG inducer concentration was 1 mM IPTG. For expression of orthogonal rRNA, the aTc inducer concentration was 100 ng/mL.

Calculation of % wild-type luciferase (η). In order to robustly compare toxic and non-toxic qtRNAs, all luminescence values are considered at OD₆₀₀=0.5 to account for differential growth rates. The % of triplet codon translation efficiency, η, is calculated using the formula:

$$η = \frac{{QuadLux}_{qtRNA induced} - {QuadLux}_{qtRNA uninduced}}{TriLux - {QuadLux}_{qtRNA uninduced}} \times 100$$

Where TriLux is the luminescence of the positive control, a luciferase encoded entirely with triplet codons; QuadLux_{qtRNA induced} is the luminescence produced by the quadruplet codon-bearing reporter upon qtRNA expression (1 mM IPTG); QuadLux_{qtRNA uninduced} is the luminescence produced by the quadruplet codon-bearing reporter upon qtRNA expression (0 mM IPTG).

Doubling time analyses. Colonies transformed with the appropriate wild-type tRNA, qtRNA, or a combination therein were picked and grown in DRM containing maintenance antibiotics. Following overnight growth at 37° C. with shaking at 900 RPM, cultures were back diluted 100-fold into DRM containing maintenance antibiotics+/−IPTG. After growing for 1 hour at 37° C. with shaking at 900 RPM in a 96 deep well plate, 175 μl of each culture were transferred to a 96-well black wall, clear bottom plate (Costar) and OD₆₀₀ was measured every 10 min over 10 hr. The doubling time of wild type and qtRNA cultures were calculated using the Growthcurver package (version 0.3.1)⁸⁴ in R (version 4.0.3).

Fluorescence assays. S3489 cells were transformed with the sfGFP and/or mCherry-based activity reporter and qtRNA expression plasmid(s). For assays containing two plasmids (one qtRNA expression plasmid and one reporter plasmid), colonies were picked directly into DRM media supplemented with maintenance antibiotics, with or without 1 mM IPTG inducer and allowed to grow overnight. For assays containing greater than two plasmids, overnight cultures of single colonies grown in DRM media supplemented with maintenance antibiotics were diluted 500-fold into DRM media with maintenance antibiotics in a 96-well 2 mL deep well plate, with or without 1 mM IPTG inducer. For three plasmid assays, the concentration of each antibiotic was cut by one third (i.e. carbenicillin, 16.7 μg/mL) and for four plasmid assays, the concentration of each antibiotic was cut by one fourth (i.e. carbenicillin 12.5 μg/mL). In all cases, the deep well plates were sealed with a porous sealing film and grown at 37° C. with shaking at 230 rpm for 24-36 h. 150 μL of cells were transferred to a 96-well black-walled clear-bottom plate, and then 600 nm absorbance and fluorescence (sfGFP: λEx=485 nm and λEm=510 nm; mCherry: λEx=587 nm and λEm=610 nm) were read at 37° C. using a Spark (Tecan) plate reader running SparkControl v2.3.

Calculation of percent wild-type sfGFP. After blank media subtraction, the percent of sfGFP triplet codon translation efficiency is calculated using the formula:

$$\% wildtype sfGFP = \frac{{Quad}_{sfGFP} / {Quad}_{OD 600}}{Average ( {wildtype}_{sfGFP} / {wildtype}_{OD 600} )} \times 100$$

Where wild-type refers to the positive control sfGFP containing no quadruplet codons; QuadsfGFP refers to fluorescence produced by the quadruplet codon-bearing reporter upon qtRNA expression (1 mM IPTG); OD600 and sfGFP values are normalized to blank media first. Threshold calculations refer to the average of fluorescence produced by the quadruplet codon-bearing reporter upon qtRNA expression (0 mM IPTG).

Sample preparation for quantification of qtRNA charging using mass spectrometry. Each qtRNA was co-expressed with C-terminal 6× His-tagged sfGFP with the appropriate quadruplet codon replacing permissive residue Y151⁵⁶ in S3489 cells. Bacterial cultures between 4 and 50 mL were grown for 36 h at 37° C. in DRM media containing IPTG inducer and appropriate antibiotics. Cultures were then pelleted and frozen at −80° C. for at least 1 day. Once thawed and weighed, appropriate volumes of complete, EDTA-free Protease Inhibitor Cocktail (1 tablet per 50 mL extraction solution; Millipore Sigma) and B-PER Bacterial Protein Extraction Reagent (4 mL per gram pellet; Thermo Fisher Scientific) were added to the cell pellet and gently pipetted up and down till homogenous. Samples were incubated for 1 hour rotating at room temperature and centrifuged at 16,000× RCF for 20 min to separate soluble proteins (supernatant from insoluble proteins (pellet). Soluble proteins were purified using either Ni-NTA spin column (Qiagen) or His-Spin Protein Miniprep (Zymo Research) and eluted in 150 μL. Resultant purified His-tagged proteins were denatured for 5 minutes at 95° C., and 22 μL sample was mixed with 7.5 μL 4× NuPAGE dye and 0.5 μL 1 M DTT. The resulting samples and Blue Prestained Protein Standard (New England Biolabs) were run on a 12% Bis-Tris PAGE gel (Invitrogen) at 200 mA, for 15 min at 90 V and then 35 min at 200 V, using 1× NuPAGE MES SDS Running Buffer (Thermo Fisher Scientific). The SDS-PAGE gel was then washed in DI H₂O for 5 min three times, stained for 2 hours in GelCode Blue Stain Reagent (Thermo Fisher Scientific), and destained in 50% methanol/water overnight. GelCode Blue stained SDS-PAGE gel lanes were then cut into ˜2 mm squares, washed once more with 47.5/47.5/5% methanol/water/acetic acid for 2 h, dehydrated with acetonitrile and dried in a speed-vac. Reduction of disulfide bonds was then carried out by the addition of 30 μl 10 mM dithiothreitol (DTT) in 100 mM ammonium bicarbonate for 30 min. The resulting free cysteine residues were subjected to an alkylation reaction by removal of the DTT solution and the addition of 100 mM iodoacetamide in 100 mM ammonium bicarbonate for 30 min to form carbamidomethyl cysteine. These were then sequentially washed with aliquots of acetonitrile, 100 mM ammonium bicarbonate and acetonitrile and dried in a speed-vac. The bands were enzymatically digested by the addition of 300 ng of trypsin (or chymotrypsin for arginine or lysine qtRNAs) in 50 mM ammonium bicarbonate to the dried gel pieces for 10 min on ice. Depending on the volume of acrylamide, excess ammonium bicarbonate was removed or enough was added to rehydrate the gel pieces. These were allowed to digest overnight at 37° C. with gentle shaking. The resulting peptides were extracted by the addition of 50 μL (or more if needed to produce supernatant) of 50 mM ammonium bicarbonate with gentle shaking for 10 min. The supernatant from this was collected in a 0.5 ml conical autosampler vial. Two subsequent additions of 47.5/47/5/5 acetonitrile/water/formic acid with gentle shaking for 10 min were performed with the supernatant added to the 0.5 mL autosampler vial. Organic solvent was removed, and the volumes were reduced to 15 μL using a speed vac for subsequent analyses.

Chromatographic separations. The digestion extracts were analyzed by reverse phase high performance liquid chromatography (HPLC) using Waters NanoAcquity pumps and autosampler and a ThermoFisher Orbitrap Elite mass spectrometer using a nano flow configuration. A 20 mm×180 μm column packed with 5 μm Symmetry C18 material (Waters) using a flow rate of 15 μl per min for 3 min was used to trap and wash peptides. These were then eluted onto the analytical column which was self-packed with 3.6 μm Aeris C18 material (Phenomenex) in a fritted 20 cm×75 μm fused silica tubing pulled to a 5 μm tip. Elution was carried out with a gradient of isocratic 1% Buffer A (1% formic acid in H₂O) for 1 min (250 nL min⁻¹), followed by increasing Buffer B (1% formic acid in acetonitrile) concentrations to 15% B at 20.5 min, 27% B at 31 min and 40% B at 36 min. The column was washed with high percent B and re-equilibrated between analytical runs for a total cycle time of approximately 53 min.

Mass spectrometry. The mass spectrometer was operated in a dependent data acquisition mode where the 10 most abundant peptides detected in the Orbitrap Elite (ThermoFisher) using full scan mode with a resolution of 240,000 were subjected to daughter ion fragmentation in the linear ion trap. A running list of parent ions was tabulated to an exclusion list to increase the number of peptides analyzed throughout the chromatographic run. The resulting fragmentation spectra were correlated against custom databases using PEAKS Studio X (Bioinformatics Solutions). To calculate the limit of detection and relative amino acid abundance, the results were matched to a library of GFP variants with each of the 20 canonical amino acids at respective residues. Abundance of each species was quantified by calculating the area under the curve of the ion chromatogram for each peptide precursor. The limit of detection was 10⁴ (arbitrary units), the lower limit for area under the curve for a peptide on this instrument.

Phage supernatant filtration. To filter 500 μL of phage, bacteria were pelleted by centrifugation at 8,000× RCF for 2 min in a tabletop centrifuge. Supernatant was transferred to a 0.22 μm filter column and centrifuged at 1000× RFF for 1 min to create filtered phage flow-through. To filter 50 mL of phage supernatant, 50 mL of culture was similarly pelleted. Supernatant was applied to a Steriflip (Millipore Sigma) 0.22 μm vacuum filter unit. To filter up to 150 μL of phage in 96-well plate format, the 96-well plate of bacteria was pelleted by centrifugation at 1,000× RCF for 10 min. 150 μL of supernatant was applied to wells of a 96-well 0.22 μm filter plate taped atop a 96-well PCR plate, and centrifuged at 1000× RCF for 1 min to create filtered phage flow-through. Phage can be stored at 4° C. in 96-well plate format covered with an aluminum sealing film. For frequently-accessed phage samples, Applicants recommend storage in 2 mL screw cap tubes in order to minimize potential phage contamination generated from opening snap-caps.

Standard phage cloning. Competent E. coli S3489 cells were prepared (as described) containing pJC175e, a plasmid expressing pIII under control of the phage shock promoter⁸⁵. To clone ΔpIII M13 bacteriophage, PCR fragments were assembled using USER, as above. The annealed fragments were transformed into competent S3489-pJC175e competent cells (as described), which complement pIII deletion from the bacteriophage. Transformants were recovered in 2× YT media overnight, shaking at 230 RPM at 37° C. The phage supernatant from the resulting culture was filtered (as described), and plaqued (as described). Clonal plaques were expanded overnight, filtered, and Sanger sequenced.

Phage library cloning. Applicants do not recommend USER cloning for library creation inside of high-secondary structure tRNAs; instead, Applicants used degenerate primers and blunt end ligation. Primers were designed containing a NNNN degenerate anticodon. To reduce nucleotide bias during blunt end ligation assembly, the last degenerate base was designed to be at least one base away from the end of the primer. For each library, 200 μL of PCR product was used. The entirety of this PCR product was run on a gel, extracted, and purified using spin column purification. Background plasmid was digested using DpnI (New England Biolabs), and the remaining PCR product was purified again using spin columns, and ligated. The ligation product was transformed into competent E. coli S3489 cells containing pJC175e. Transformants were recovered in 2× YT media overnight, shaking at 230 RPM at 37° C. The phage supernatant from the resulting culture was filtered and plaqued.

Manual phage plaque assays. S3489 cells were transformed with the Accessory Plasmid of interest. Overnight cultures of single colonies grown in 2× YT media supplemented with maintenance antibiotics were diluted 1,000-fold into fresh 2× YT media with maintenance antibiotics and grown at 37° C. with shaking at 230 rpm to OD₆₀₀ ˜0.6-0.8 before use. Bacteriophage were serially diluted 100-fold (4 dilutions total) in H₂O. 100 μL of cells were added to 100 μL of each phage dilution, and to this 0.85 mL of liquid (70° C.) top agar (2× YT media+0.6% agar) supplemented with 2% Bluo-Gal was added and mixed by pipetting up and down once. This mixture was then immediately pipetted onto one quadrant of a quartered Petri dish already containing 2 mL of solidified bottom agar (2× YT media+1.5% agar). After solidification of the top agar, plates were incubated at 37° C. for 16-18 h.

Phage enrichment assays. S3489 cells were transformed with the Accessory Plasmids of interest as described above. Overnight cultures of single colonies grown in 2× YT media supplemented with maintenance antibiotics were diluted 1,000-fold into DRM media with maintenance antibiotics and grown at 37° C. with shaking at 230 RPM to OD₆₀₀ ˜0.4-0.6. Cells were then infected with bacteriophage at a starting titer of 105 pfu/mL. Cells were incubated for another 16-18 h at 37° C. with shaking at 230 RPM. Supernatant was filtered and stored at 4° C. The phage titer of these samples was measured in an activity-independent manner using a plaque assay containing E. coli bearing pJC175e.

Continuous flow PACE. Unless otherwise noted, PACE apparatus, including host cell strains, lagoons, chemostats, and media, were all used as previously described⁶⁶. Chemically competent S3489 cells were transformed with the Accessory Plasmid and the mutagenesis plasmid (MP) MP635 as described above, plated on 2× YT media+1.5% agar supplemented with 25 mM glucose (to prevent induction of mutagenesis) in addition to maintenance antibiotics, and grown at 37° C. for 18-20 h. To validate MP functionality prior to evolution, four colonies were picked into 10 μL DRM media and diluted 10-fold six times; these dilutions were plated on either agar plates with maintenance antibiotics and 25 mM glucose, or 10 mM arabinose; Applicants expected that colonies plated on arabinose would be of reduced size when the MP is functional. The remainder of the dilutions were added to 2 mL DRM media with maintenance antibiotics, grown at 37° C. with shaking until they reached OD₆₀₀ ˜0.4-0.8, and then used to inoculate a turbidostat containing 30 mL DRM media. The turbidostat maintained the growing culture at OD₆₀₀ ˜0.7-0.8. Prior to bacteriophage infection, lagoons were continuously diluted with culture from the turbidostat at 1 lagoon vol/h and pre-induced with 10 mM arabinose for at least 45 min to induce mutagenesis. Samples (500 μL.) of the SP population were taken at indicated times from lagoon waste lines. These were centrifuged at 8,000 RCF for 2 min, and the supernatant was passed through a 0.22 μm filter and stored at 4° C. Lagoon titers were determined by plaque assays using S3489 cells transformed with pJC175e.

Aminoacyl-tRNA synthetase expression and purification. E. coli SerRS, ArgRS, and TyrRS were overexpressed in BL21 (DE3) E. coli cells and purified as previously described³⁵ with slight modifications. Cells were grown at 37° C. until OD₆₀₀ 0.6 and induced with 0.5 mM isopropyl β-D-1-thiogalactopyranoside for 3 hours at 30° C. Cells were resuspended in Buffer A (50 mM HEPES-KOH [pH 7.5], 300 mM NaCl, 10 mM β-mercaptoethanol, 3 mM MgCl₂, 10 mM imidazole) along with a protease inhibitor tablet (Roche, complete Mini, EDTA-free) and subjected to sonication. The lysate was centrifuged at 38 000 RPM for 40 minutes at 4° C. and the synthetases were purified via nickel affinity chromatography. The synthetases were eluted with Buffer B (50 mM HEPES-KOH [pH 7.5], 300 mM NaCl, 10 mM β-mercaptoethanol, 3 mM MgCl₂, 250 mM imidazole) and incubated with His-tagged TEV protease for 1 hour at 37° C. The aaRS-TEV protease solution was dialyzed into Buffer A, subjected to nickel affinity chromatography to isolate the aaRS, dialyzed into a storage buffer (50 mM HEPES-KOH [pH 7.5], 100 mM NaCl, 10 mM β-mercaptoethanol, 3 mM MgCl₂, 50% glycerol), and stored at −80° C.

In vitro aminoacylation assay. All qtRNAs were in vitro transcribed using T7 RNA polymerase and gel purified as previously described³⁵. Prior to use, the qtRNAs were heated to 85° C. and slowly cooled down to room temperature in the presence of 10 mM MgCl₂ to allow proper refolding. In vitro aminoacylation of tRNA^{Ser}_{UAGA} by E. coli seryl-tRNA synthetase, tRNA^{Arg}_{UAGA} by E. coli arginyl-tRNA synthetase, and tRNA^{Tyr}_{UAGA} by E. coli tyrosyl-tRNA synthetase were performed as previously described⁶⁵. Briefly, reactions contained 50 mM HEPES-KOH [pH 7.3], 4 mM ATP, 25 mM MgCl₂, 0.1 mg/mL bovine serum albumin, 20 mM KCl, 20 mM 2-mercaptoethanol, 4 μM qtRNA, amino acid (25 μM L-[¹⁴C]-Ser, 6 μM L-Arg (2 μM L-[¹⁴C]-Arg, 4 μM L-Arg), or 6 μM L-Tyr (2 μM L-[¹⁴C]-Tyr, 4 μM L-Tyr)) and E. coli aaRS (50 mM SerRS, 30 nM ArgRS, or 30 nM TyrRS). The reactions were incubated at 37° C. and 8 μL aliquots were removed at given intervals, spotted onto 3 MM filter papers (presoaked with 5% trichloroacetic acid and dried), immersed in 5% TCA to precipitate aminoacyl-qtRNAs, and then subjected to scintillation counting.

tRNA diagrams. R2R was used to generate tRNA diagrams. R2R is free software available from sourceforge.net/projects/weinberg-r2r/.

### REFERENCES

1. Furter, R. Expansion of the genetic code: site-directed p-fluoro-phenylalanine incorporation in Escherichia coli. Protein Sci 7, 419-426 (1998).
2. Link, A.J., Mock, M.L. & Tirrell, D.A. Non-canonical amino acids in protein engineering. Curr Opin Biotechnol 14, 603-609 (2003).
3. Mukai, T., Lajoie, M.J., Englert, M. & Söll, D. Rewriting the genetic code. Annu Rev Microbiol 71, 557-577 (2017).
4. Chin, J.W. Expanding and reprogramming the genetic code. Nature 550, 53-60 (2017).
5 Ho, J.M. et al. Efficient Reassignment of a Frequent Serine Codon in Wild-Type Escherichia coli. ACS Synth Biol 5, 163-171 (2016).
6. Robertson, W.E. et al. Sense codon reassignment enables viral resistance and encoded polymer synthesis. Science 372, 1057-1062 (2021).
7. Bossi, L. & Roth, J.R. Four-base codons ACCA, ACCU and ACCC are recognized by frameshift suppressor sufJ. Cell 25, 489-496 (1981).
8. Magliery, T.J., Anderson, J.C. & Schultz, P.G. Expanding the genetic code: selection of efficient suppressors of four-base codons and identification of “shifty” four-base codons with a library approach in Escherichia coli. J Mol Biol 307, 755-769 (2001).
9. Anderson, J.C. et al. An expanded genetic code with a functional quadruplet codon. Proc Natl Acad Sci USA 101, 7566-7571 (2004).
10. Hohsaka, T., Ashizuka, Y., Taira, H., Murakami, H. & Sisido, M. Incorporation of nonnatural amino acids into proteins by using various four-base codons in an Escherichia coli in vitro translation system. Biochemistry 40, 11060-11064 (2001).
11. Neumann, H., Wang, K., Davis, L., Garcia-Alai, M. & Chin, J.W. Encoding multiple unnatural amino acids via evolution of a quadruplet-decoding ribosome. Nature 464, 441-444 (2010).
12. Moore, B., Persson, B.C., Nelson, C.C., Gesteland, R.F. & Atkins, J.F. Quadruplet codons: implications for code expansion and the specification of translation step size. J Mol Biol 298, 195-209 (2000).
13. Malyshev, D.A. et al. A semi-synthetic organism with an expanded genetic alphabet. Nature 509, 385-388 (2014).
14. Hoshika, S. et al. Hachimoji DNA and RNA: A genetic system with eight building blocks. Science 363, 884-887 (2019).
15. Liu, C.C. & Schultz, P.G. Adding new chemistries to the genetic code. Annu Rev Biochem 79, 413-444 (2010).
16. Wan, W., Tharp, J.M. & Liu, W.R. Pyrrolysyl-tRNA synthetase: an ordinary enzyme but an outstanding genetic code expansion tool. Biochim Biophys Acta 1844, 1059-1070 (2014).
17. Melo Czekster, C., Robertson, W.E., Walker, A.S., Söll, D. & Schepartz, A. In Vivo Biosynthesis of a beta-Amino Acid-Containing Protein. J Am Chem Soc 138, 5194-5197 (2016).
18. Ad, O. et al. Translation of Diverse Aramid- and 1,3-Dicarbonyl-peptides by Wild Type Ribosomes in Vitro. ACS Cent Sci 5, 1289-1294 (2019).
19. Terasaka, N., Iwane, Y., Geiermann, A.S., Goto, Y. & Suga, H. Recent developments of engineered translational machineries for the incorporation of non-canonical amino acids into polypeptides. Int J Mol Sci 16, 6513-6531 (2015).
20. Italia, J.S. et al. Mutually Orthogonal Nonsense-Suppression Systems and Conjugation Chemistries for Precise Protein Labeling at up to Three Distinct Sites. J Am Chem Soc 141, 6204-6212 (2019).
21. Roth, J.R. Frameshift suppression. Cell 24, 601-602 (1981).
22. Gamper, H. et al. Insights into genome recoding from the mechanism of a classic +1-frameshifting tRNA. Nat Commun 12, 328 (2021).
23. Anderson, J.C., Magliery, T.J. & Schultz, P.G. Exploring the limits of codon and anticodon size. Chem Biol 9, 237-244 (2002).
24. Atkins, J.F. & Bjork, G.R. A gripping tale of ribosomal frameshifting: extragenic suppressors of frameshift mutations spotlight P-site realignment. Microbiol Mol Biol Rev 73, 178-210 (2009)
25. Bossi, L. & Smith, D.M. Suppressor sufJ: a novel type of tRNA mutant that induces translational frameshifting. Proc Natl Acad Sci USA 81, 6105-6109 (1984).
26. Curran, J.F. & Yarus, M. Reading frame selection and transfer RNA anticodon loop stacking. Science 238, 1545-1550 (1987).
27. O'Connor, M. Insertions in the anticodon loop of tRNA1Gln (sufG) and tRNA (Lys) promote quadruplet decoding of CAAA. Nucleic Acids Res 30, 1985-1990 (2002).
28. Giege, R., Sissler, M. & Florentz, C. Universal rules and idiosyncratic features in tRNA identity. Nucleic Acids Res 26, 5017-5035 (1998).
29. LaRiviere, F.J., Wolfson, A.D. & Uhlenbeck, O.C. Uniform binding of aminoacyl-tRNAs to elongation factor Tu by thermodynamic compensation. Science 294, 165-168 (2001).
30. Yarus, M., Valle, M. & Frank, J. A twisted tRNA intermediate sets the threshold for decoding. RNA 9, 384-385 (2003).
31. DeLey Cox, V.E., Cole, M.F. & Gaucher, E.A. Incorporation of Modified Amino Acids by Engineered Elongation Factors with Expanded Substrate Capabilities. ACS Synth Biol 8, 287-296 (2019).
32. Hong, S. et al. Mechanism of tRNA-mediated +1 ribosomal frameshifting. Proc Natl Acad Sci USA 115, 11226-11231 (2018).
33. Wang, N., Shang, X., Cerny, R., Niu, W. & Guo, J. Systematic Evolution and Study of UAGN Decoding tRNAs in a Genomically Recoded Bacteria. Sci Rep 6, 21898 (2016).
34. Hankore, E.D. et al. Genetic Incorporation of Noncanonical Amino Acids Using Two Mutually Orthogonal Quadruplet Codons. ACS Synth Biol 8, 1168-1174 (2019).
35. Bryson, D.I. et al. Continuous directed evolution of aminoacyl-tRNA synthetases. Nat Chem Biol 13, 1253-1260 (2017).
36. Carlson, J.C., Badran, A.H., Guggiana-Nilo, D.A. & Liu, D.R. Negative selection and stringency modulation in phage-assisted continuous evolution. Nat Chem Biol 10, 216-222 (2014).
37. Ryu, Y.H. & Schultz, P.G. Efficient incorporation of unnatural amino acids into proteins in Escherichia coli. Nat Methods 3, 263-265 (2006).
38. Perret, V. et al. Relaxation of a transfer RNA specificity by removal of modified nucleotides. Nature 344, 787-789 (1990).
39. Asahara, H. et al. Recognition nucleotides of Escherichia coli tRNA (Leu) and its elements facilitating discrimination from tRNASer and tRNA (Tyr). J Mol Biol 231, 219-229 (1993).
40. Muramatsu, T. et al. Codon and amino-acid specificities of a transfer RNA are both converted by a single post-transcriptional modification. Nature 336, 179-181 (1988).
41. Pagel, F.T. & Murgola, E.J. A base substitution in the amino acid acceptor stem of RNA (Lys) causes both misacylation and altered decoding. Gene Expr 6, 101-112 (1996).
42. Park, S.J. & Schimmel, P. Evidence for interaction of an aminoacyl transfer RNA synthetase with a region important for the identity of its cognate transfer RNA. J Biol Chem 263, 16527-16530 (1988).
43. Asahara, H. et al. Discrimination among E. coli tRNAs with a long variable arm. Nucleic Acids Symp Ser, 207-208 (1993).
44. Dock-Bregeon, A.C., Garcia, A., Giege, R. & Moras, D. The contacts of yeast tRNA (Ser) with seryl-tRNA synthetase studied by footprinting experiments. Eur J Biochem 188, 283-290 (1990).
45. Jahn, M., Rogers, M.J. & Soll, D. Anticodon and acceptor stem nucleotides in tRNA (Gln) are major recognition elements for E. coli glutaminyl-tRNA synthetase. Nature 352, 258-260 (1991).
46. Yan, W., Augustine, J. & Francklyn, C. A tRNA identity switch mediated by the binding interaction between a tRNA anticodon and the accessory domain of a class II aminoacyl-tRNA synthetase. Biochemistry 35, 6559-6568 (1996).
47. Cupples, C.G. & Miller, J.H. Effects of amino acid substitutions at the active site in Escherichia coli beta-galactosidase. Genetics 120, 637-644 (1988).
48. Wheatley, R.W. et al. Substitution for Asn460 cripples beta-galactosidase (Escherichia coli) by increasing substrate affinity and decreasing transition state stability. Arch Biochem Biophys 521, 51-61 (2012).
49. Xu, J., McRae, M.A., Harron, S., Rob, B. & Huber, R.E. A study of the relationships of interactions between Asp-201, Na+ or K+, and galactosyl C6 hydroxyl and their effects on binding and reactivity of beta-galactosidase. Biochem Cell Biol 82, 275-284 (2004).
50. Huber, R.E., Hlede, I.Y., Roth, N.J., Mckenzie, K.C. & Ghumman, K.K. His-391 of beta-galactosidase (Escherichia coli) promotes catalyses by strong interactions with the transition state. Biochem Cell Biol 79, 183-193 (2001).
51. Roth, N.J. & Huber, R.E. The beta-galactosidase (Escherichia coli) reaction is partly facilitated by interactions of His-540 with the C6 hydroxyl of galactose. Journal of Biological Chemistry 271, 14296-14301 (1996).
52. Akaboshi, E., Inouye, M. & Tsugita, A. Effect of neighboring nucleotide sequences on suppression efficiency in amber mutants of T4 phage lysozyme. Mol Gen Genet 149, 1-4 (1976).
53. Fluck, M.M. & Epstein, R.H. Isolation and characterization of context mutations affecting the suppressibility of nonsense mutations. Mol Gen Genet 177, 615-627 (1980).
54. Salser, W., Fluck, M. & Epstein, R. The influence of the reading context upon the suppression of nonsense codons. 3. Cold Spring Harb Symp Quant Biol 34, 513-520 (1969).
55. O'Donoghue, P. et al. Near-cognate suppression of amber, opal and quadruplet codons competes with aminoacyl-tRNAPyl for genetic code expansion. FEBS Lett 586, 3931-3937 (2012).
56. Young, T.S., Ahmad, I., Yin, J.A. & Schultz, P.G. An enhanced system for unnatural amino acid mutagenesis in E. coli. J Mol Biol 395, 361-374 (2010).
57. Zhang, S.P., Zubay, G. & Goldman, E. Low-usage codons in Escherichia coli, yeast, fruit fly and primates. Gene 105, 61-72 (1991).
58. Chatterjee, A., Lajoie, M.J., Xiao, H., Church, G.M. & Schultz, P.G. A bacterial strain with a unique quadruplet codon specifying non-native amino acids. Chembiochem 15, 1782-1786 (2014).
59. Lajoie, M.J. et al. Genomically recoded organisms expand biological functions. Science 342, 357-360 (2013).
60. Wannier, T.M. et al. Adaptive evolution of genomically recoded Escherichia coli. Proc Natl Acad Sci USA 115, 3090-3095 (2018).
61. Aerni, H.R., Shifman, M.A., Rogulina, S., O'Donoghue, P. & Rinehart, J. Revealing the amino acid composition of proteins within an expanded genetic code. Nucleic Acids Res 43, e8 (2015).
62. Hughes, R.A. & Ellington, A.D. Rational design of an orthogonal tryptophanyl nonsense suppressor tRNA. Nucleic Acids Res 38, 6813-6830 (2010).
63. Kleina, L.G., Masson, J.M., Normanly, J., Abelson, J. & Miller, J.H. Construction of Escherichia coli amber suppressor tRNA genes. II. Synthesis of additional tRNA genes and improvement of suppressor efficiency. J Mol Biol 213, 705-717 (1990).
64. Yarus, M. Translational efficiency of transfer RNA's: uses of an extended anticodon. Science 218, 646-652 (1982).
65. Salazar, J.C. et al. Coevolution of an aminoacyl-tRNA synthetase with its tRNA substrates. Proc Natl Acad Sci USA 100, 13863-13868 (2003).
66. Esvelt, K.M., Carlson, J.C. & Liu, D.R. A system for the continuous directed evolution of biomolecules. Nature 472, 499-U550 (2011).
67. Badran, A.H. & Liu, D.R. Development of potent in vivo mutagenesis plasmids with broad mutational spectra. Nat Commun 6, 8425 (2015).
68. Wang, L., Magliery, T.J., Liu, D.R. & Schultz, P.G. A new functional suppressor tRNA/aminoacyl-tRNA synthetase pair for the in vivo incorporation of unnatural amino acids into proteins. J Amer Chem Soc 122, 5010-5011 (2000).
69. Johnson, D.B.F. et al. RF1 knockout allows ribosomal incorporation of unnatural amino acids at multiple sites. Nature Chemical Biology 7, 779-786 (2011).
70. Ibba, M. & Söll, D. Aminoacyl-tRNA synthesis. Annh Rev Biochem 69, 617-650 (2000).
71. Jahn, M., Rogers, M.J. & Söll, D. Anticodon and acceptor stem nucleotides in transfer RNA^{Gln} are major recognition elements for Escherichia coli glutaminyl-transfer RNA-synthetase. Nature 352, 258-260 (1991).
72. Rogers, M.J., Adachi, T., Inokuchi, H. & Söll, D. Switching tRNAGIn identity from glutamine to tryptophan. Proc Natl Acad Sci USA 89, 3463-3467 (1992).
73. Rould, M.A., Perona, J.J., Soll, D. & Steitz, T.A. Structure of E. coli glutaminyl-tRNA synthetase complexed with tRNA (Gln) and ATP at 2.8 A resolution. Science 246, 1135-1142 (1989).
74. Rodriguez-Hernandez, A. et al. Structural and mechanistic basis for enhanced translational efficiency by 2-thiouridine at the tRNA anticodon wobble position. J Mol Biol 425, 3888-3906 (2013).
75. Fagan, C.E., Maehigashi, T., Dunkle, J.A., Miles, S.J. & Dunham, C.M. Structural insights into translational recoding by frameshift suppressor tRNA (SufJ). Rna 20, 1944-1954 (2014).
76. Baker, S.L. & Hogg, J.R. A system for coordinated analysis of translational readthrough and nonsense-mediated mRNA decay. PLoS One 12, e0173980 (2017).
77. Fan, Y. et al. Heterogeneity of Stop Codon Readthrough in Single Bacterial Cells and Implications for Population Fitness. Mol Cell 67, 826-836 e825 (2017).
78. Dunkelmann, D.L., Willis, J.C.W., Beattie, A.T. & Chin, J.W. Engineered triply orthogonal pyrrolysyl-tRNA synthetase/tRNA pairs enable the genetic encoding of three distinct non-canonical amino acids. Nat Chem 12, 535-544 (2020).
79. Namy, O. et al. Adding pyrrolysine to the Escherichia coli genetic code. FEBS Lett 581, 5282-5288 (2007).
80. Niu, W., Schultz, P.G. & Guo, J. An expanded genetic code in mammalian cells with a functional quadruplet codon. ACS Chem Biol 8, 1640-1645 (2013).
81. Riddle, D.L. & Carbon, J. Frameshift suppression: a nucleotide addition in the anticodon of a glycine transfer RNA. Nat New Biol 242, 230-234 (1973).
82. Hubbard, B.P. et al. Continuous directed evolution of DNA-binding proteins to improve TALEN specificity. Nat Methods 12, 939-942 (2015).
83. Li, Z., Gong, X., Joshi, V.H. & Li, M. Co-evolution of tRNA 3′ trailer sequences with 3′ processing enzymes in bacteria. RNA 11, 567-577 (2005).
84. Sprouffske, K. & Wagner, A. Growthcurver: an R package for obtaining interpretable metrics from microbial growth curves. BMC Bioinformatics 17, 172 (2016).
85. Badran, A.H. et al. Continuous evolution of Bacillus thuringiensis toxins overcomes insect resistance. Nature 533, 58-63 (2016).
86. Lajoie, M.J. et al. Genomically recoded organisms expand biological functions. Science 342, 357-360 (2013).
87. Johnson, D.B.F. et al. RF1 knockout allows ribosomal incorporation of unnatural amino acids at multiple sites. Nature Chemical Biology 7, 779-786 (2011).
88. Neumann, H., Wang, K., Davis, L., Garcia-Alai, M. & Chin, J.W. Encoding multiple unnatural amino acids via evolution of a quadruplet-decoding ribosome. Nature 464, 441-444 (2010).

### Tables

TABLE 1
Previously reported quadruplet-decoding tRNAs discovered in bacterial isolates.
Spontaneous mutations in the tRNA which expand the anticodon by 1 base enable the decoding
of quadruplet codons. Differences between the natural codon and the suppressed quadruplet
codon are shown in bold. AA: amino acid.

| | | | | Sup- | Suppressed | | |
|---|---|---|---|---|---|---|---|
| Class | AA | Gene | Codon | pressor | Codon | Source Organism | Reference |
| Elongator | Leu | leuX | UUG | su6 | UAGN | Escherichia coli | Moore 2000 |
| Elongator | Val | valU | GUU | hopR1 | GUUA | Escherichia coli | O'Connor 1989 |
| Elongator | Val | valU | GUA | hopR513 | GUAA | Escherichia coli | O'Connor 1989 |
| Elongator | Gln | trpT | UGG | su7 | UAGN | Escherichia coli | Curran 1987 |
| Elongator | Gly | glyU | GGG | sufD | GGGG | Salmonella typhimurium | Riddle 1973 |
| Elongator | Pro | proL | CCC | sufB | CCCC | Salmonella typhimurium | Sroga 1992 |
| Elongator | Gln | glnW | CAA | sufG | CAAA | Salmonella typhimurium | O'Connor 2002 |
| Elongator | Thr | thrT | ACC | sufJ | ACCH | Salmonella typhimurium | Bossi 1984 |
| Elongator | Gly | SUF16 | GGC | suf16 | GGGC | Saccharomyces cerevisiae | Gaber 1982 |

TABLE 2
Sequences of all natural E. coli tRNA scaffolds used for qtRNA engineering. In all cases,
tRNA sequences are shown in bold, and the anticodon is underlined. Flanking sequences were
included in vector design to ensure efficient qtRNA maturation. All coordinates derive from E.
coli DH10B genome.

| | | | | E. coli DH10B | |
|---|---|---|---|---|---|
| | Amino | | | coordinates | |
| Class | Acid | Gene | Sequence (tRNA, anticodon) | Start | End |
| Elongator | A | alaX | --------------- | 2607923 | 2607808 |
| | | | TTGGTACGTAAACGCATCGTGGGGCTATAGCTCAGCTGGGAGAGCGCTTGCATGGCA | | |
| | | | TGCAAGAGGTCAGCGGTTCGATCCCGCTTAGCTCCACCAAATTTCCAACCCTCGCTGCA | | |
| | | | -------------------- (SEQ ID NO: 1) | | |
| Elongator | C | cysT | --------------- | 2081039 | 2080926 |
| | | | CTGAAAGGCCTGAAGAATTTGGCGCGTTAACAAAGCGGTTATGTAGCGGATTGCAAA | | |
| | | | TCCGTCTAGTCCGGTTCGACTCCGGAACGCGCCTCCACTTTCTTCCCGAGCCCGGAT---- | | |
| | | | -------------------- (SEQ ID NO: 2) | | |
| Elongator | D | aspU | --------------- | 203012 | 203128 |
| | | | TGGTTGTAAAAGAATTCGGTGGAGCGGTAGTTCAGTCGGTTAGAATACCTGCCTGTCA | | |
| | | | CGCAGGGGGTCGCGGGTTCGAGTCCCGTCCGTTCCGCCACTTATTAAGAAGCCTCGAG | | |
| | | | T------------------- (SEQ ID NO: 3) | | |
| Elongator | E | gltT | --------------- | 2819251 | 2819136 |
| | | | GCGTGATAAGCAATTTTCGTGTCCCCTTCGTCTAGAGGCCCAGGACACCGCCCTTTCAC | | |
| | | | GGCGGTAACAGGGGTTCGAATCCCCTAGGGGACGCCACTTGCTGGTTTGTGAGTGAA- | | |
| | | | -------------------- (SEQ ID NO: 4) | | |
| Elongator | F | pheV | --------------- | 3207951 | 3207836 |
| | | | CAGGTTTAATGCGCCCCGTTGCCCGGATAGCTCAGTCGGTAGAGCAGGGGATTGAAA | | |
| | | | ATCCCCGTGTCCTTGGTTCGATTCCGAGTCCGGGCACCACTAATTCTTAAGAACCCGCC | | |
| | | | -------------------- (SEQ ID NO: 5) | | |
| Elongator | G | glyU | --------------- | 3090969 | 3090856 |
| | | | AATGCCTTACGCATCTCGAAGCGGGCGTAGTTCAATGGTAGAACGAGAGCTTCCCAA | | |
| | | | GCTCTATACGAGGGTTCGATTCCCTTCGCCCGCTCCAATTTATCTTCGCCGTAATAC ----- | | |
| | | | -------------------- (SEQ ID NO: 6) | | |
| Elongator | H | hisR | --------------- | 4079433 | 4079548 |
| | | | GCGTAACAAGATTTGTAGTGGTGGCTATAGCTCAGTTGGTAGAGCCCTGGATTGTGAT | | |
| | | | TCCAGTTGTCGTGGGTTCGAATCCCATTAGCCACCCCATTATTAGAAGTTGTGACAAT -- | | |
| | | | -------------------- (SEQ ID NO: 7) | | |
| Elongator | I | ileT | --------------- | 4134064 | 4134170 |
| | | | ATGAGCAGTAAAACCTCTACAGGCTTGTAGCTCAGGTGGTTAGAGCGCACCCCTGATA | | |
| | | | AGGGTGAGGTCGGTGGTTCAAGTCCACTCAGGCCTACCAAATTTGCACG- | | |
| | | | -------------------- (SEQ ID NO: 8) | | |
| Elongator | K | lysQ | ------- | 833361 | 833523 |
| | | | CTGAATGATTAAGGCAGCATAATCCCGCAAGGGGTCGTTAGCTCAGTTGGTAGAGCA | | |
| | | | GTTGACTTTTAATCAATTGGTCGCAGGTTCGAATCCTGCACGACCCACCAATGTAAAA | | |
| | | | AAGCGCCCTAAAGGCGCTTTTTTGCTATCTGCGATACTCAAAGATTCG (SEQ ID NO: 9) | | |
| Elongator | L | leuX | --------------- | 4596216 | 4596340 |
| | | | TTTCCGCATACCTCTTCAGTGCCGAAGTGGCGAAATCGGTAGACGCAGTTGATTCAAA | | |
| | | | ATCAACCGTAGAAATACGTGCCGGTTCGAGTCCGGCCTTCGGCACCAAAAGTATGTAA | | |
| | | | ATAGACCTC---------------- (SEQ ID NO: 10) | | |
| Elongator | M | metU | CCCCAGCCATCGAAGAAACAATCTGGCTACGTAGCTCAGTTGGTTAGAGCACATCACT | 748579 | 748445 |
| | | | CATAATGATGGGGTCACAGGTTCGAATCCCGTCGTAGCCACCAAATTCTGAATGTATC | | |
| | | | GAATATGTTCGGCAAATTC--------------- (SEQ ID NO: 11) | | |
| Initiator | fM | metZ | GTATAGTGCGCATCCACGGACGCGGGGTGGAGCAGCCTGGTAGCTCGTCGGGCTCAT | 3039259 | 3039375 |
| | | | AACCCGAAGGTCGTCGGTTCAAATCCGGCCCCCGCAACCAATTAAAATTTGATGAAGT | | |
| | | | AA----------------- (SEQ ID NO: 12) | | |
| Elongator | N | asnT | --------------- | 2133561 | 2133676 |
| | | | ATTCGCCCCGTTCACACGATTCCTCTGTAGTTCAGTCGGTAGAACGGCGGACTGTTAAT | | |
| | | | CCGTATGTCACTGGTTCGAGTCCAGTCAGAGGAGCCAAATTCTAAAAATTCGCTTTT--- | | |
| | | | -------------------- (SEQ ID NO: 13) | | |
| Elongator | P | proK | --------------- | 3804312 | 3804196 |
| | | | AGTAAGATGCGCCCCGCATTCGGTGATTGGCGCAGCCTGGTAGCGCACTTCGTTCGGG | | |
| | | | ACGAAGGGGTCGGAGGTTCGAATCCTCTATCACCGACCAAATTCGAAAAGCCTGCTCA | | |
| | | | A-------------------- (SEQ ID NO: 14) | | |
| Elongator | Q | glnX | --------------- | 748225 | 748339 |
| | | | ACCTTGTAAGTGCACCCAGTTGGGGTATCGCCAAGCGGTAAGGCACCGGATTCTGATT | | |
| | | | CCGGCATTCCGAGGTTCGAATCCTCGTACCCCAGCCACATTAAAAAAGCTCGCTTCG--- | | |
| | | | ---------------------- (SEQ ID NO: 15) | | |
| Elongator | R | argQ | --------------- | 2908444 | 2908328 |
| | | | GTAGAATAAGTTTTCCCGATGCATCCGTAGTTCAGCTGGATAGAGTACTCGGCTACGA | | |
| | | | ACCGAGCGGTCGGAGGTTCGAATCCTCCCGGATGCACCATATTCTCCGTAACCTTCAG | | |
| | | | C------------------- (SEQ ID NO: 16) | | |
| Elongator | S | serU | --------------- | 2132609 | 2132480 |
| | | | ATCATGGCAACCATCTGAACGGAGAGATGCCGGAGCGGCTGAACGGACCGGTCTCGA | | |
| | | | AAACCGGAGTAGGGGCAACTCTACCGGGGGTTCAAATCCCCCTCTCTCCGCCACTTTA | | |
| | | | TCAATGACTTATCTC-------------- (SEQ ID NO: 17) | | |
| Elongator | T | thrW | --------------- | 236179 | 236294 |
| | | | CGTGAACATGTCCTTTCAGGGCCGATATAGCTCAGTTGGTAGAGCAGCGCATTCGTAA | | |
| | | | TGCGAAGGTCGTAGGTTCGACTCCTATTATCGGCACCATTAAAATCAAATTGTTACGT-- | | |
| | | | ---------------- (SEQ ID NO: 18) | | |
| Elongator | V | valW | --------------- | 1835091 | 1835207 |
| | | | CCAATTGAACGCACCATCCTGCGTCCGTAGCTCAGTTGGTTAGAGCACCACCTTGACAT | | |
| | | | GGTGGGGGTCGGTGGTTCGAGTCCACTCGGACGCACCAGATTTTCTTAATCTGGTCTT- | | |
| | | | ---------------- (SEQ ID NO: 19) | | |
| Elongator | W | trpT | --------------- | 4043863 | 4043995 |
| | | | CGCGGGTTCGAGTCCCGTCCGTTCCGCCACCCTAATTAGGGGCGTAGTTCAATTGGTA | | |
| | | | GAGCACCGGTCTCCAAAACCGGGTGTTGGGAGTTCGAGTCTCTCCGCCCCTGCCAGAA | | |
| | | | ATCATCCTTAGCGAAAG- (SEQ ID NO: 20) | | |
| Elongator | Y | tyrU | --------------- | 4273171 | 4273293 |
| | | | TAGTCGGCACCATCAAGTCCGGTGGGGTTCCCGAGCGGCCAAAGGGAGCAGACTGTA | | |
| | | | AATCTGCCGTCACAGACTTCGAAGGTTCGAATCCTTCCCCCACCACCAATTTCGGCCAC | | |
| | | | GCGATGG- (SEQ ID NO: 21) | | |

TABLE 3
Doubling time analysis for all natural, engineered, and
evolved qtRNAs. All doubling time analyses used S3489
cells with tRNA expression plasmids encoding the shown
tRNA under induced conditions. Data represents the mean
and standard deviation of 4-8 biological replicates.

| | Doubling time ± |
|---|---|
| | standard deviation |
| tRNA^{Amino Acid}_{Anticodon} | (min) |
| tRNA^{Ala}_{GCC} | 21.7 ± 0.3 |
| qtRNA^{Ala}_{UAGA} | 20.4 ± 0.4 |
| tRNA^{Arg}_{CGU} | 22.8 ± 0.4 |
| qtRNA^{Arg}_{UAGA} | 21.7 ± 0.5 |
| qtRNA^{Arg}_{UAGA-Evo1} | 21.9 ± 0.3 |
| qtRNA^{Arg}_{UAGA-Evo2} | 22.0 ± 0.4 |
| tRNA^{Asn}_{AAC} | 22.3 ± 4.3 |
| qtRNA^{Asn}_{UAGA} | 25.9 ± 4.2 |
| tRNA^{Asp}_{GAC} | 20.7 ± 0.5 |
| qtRNA^{Asp}_{UAGA} | 23.2 ± 4.1 |
| tRNA^{Cys}_{UGC} | 21.0 ± 0.5 |
| qtRNA^{Cys}_{UAGA} | 22.0 ± 3.4 |
| tRNA^{Gln}_{CAG} | 24.8 ± 2.9 |
| qtRNA^{Gln}_{UAGA} | 21.5 ± 0.6 |
| qtRNA^{Gln}_{UAGA-Evo1} | 23.4 ± 0.3 |
| qtRNA^{Gln}_{UAGA-Evo2} | 21.3 ± 0.7 |
| tRNA^{Glu}_{GAA} | 24.5 ± 3.7 |
| qtRNA^{Glu}_{UAGA} | 24.5 ± 4.1 |
| tRNA^{Gly}_{GGG} | 22.0 ± 0.4 |
| qtRNA^{Gly}_{UAGA} | 24.9 ± 5.2 |
| tRNA^{His}_{CAC} | 21.7 ± 0.5 |
| qtRNA^{His}_{UAGA} | 25.0 ± 4.2 |
| tRNA^{Ile}_{AUC} | 21.6 ± 0.4 |
| qtRNA^{Ile}_{UAGA} | 22.5 ± 0.3 |
| tRNA^{Leu}_{UUA} | 23.1 ± 0.4 |
| qtRNA^{Leu}_{UAGA} | 23.5 ± 0.5 |
| tRNA^{Lys}_{AAA} | 21.1 ± 0.6 |
| qtRNA^{Lys}_{UAGA} | 23.0 ± 0.2 |
| tRNA^{fMet}_{AUG} | 19.2 ± 0.4 |
| qtRNA^{fMet}_{UAGA} | 22.2 ± 4.7 |
| tRNA^{Met}_{AUG} | 21.3 ± 0.6 |
| qtRNA^{Met}_{UAGA} | 25.0 ± 4.0 |
| tRNA^{Phe}_{AAC} | 24.4 ± 4.4 |
| qtRNA^{Phe}_{UAGA} | 24.9 ± 4.8 |
| tRNA^{Pro}_{CCG} | 24.2 ± 5.0 |
| qtRNA^{Pro}_{UAGA} | 21.4 ± 0.5 |
| tRNA^{Ser}_{UCG} | 20.3 ± 2.3 |
| qtRNA^{Ser}_{UAGA} | 21.7 ± 3.5 |
| qtRNA^{Ser}_{UAGA-Evo1} | 20.2 ± 2.2 |
| qtRNA^{Ser}_{UAGA-Evo2} | 19.4 ± 0.4 |
| qtRNA^{Ser}_{UAGA-Evo3} | 19.8 ± 1.2 |
| tRNA^{Thr}_{ACC} | 22.8 ± 4.5 |
| qtRNA^{Thr}_{UAGA} | 23.0 ± 0.5 |
| tRNA^{Trp}_{UUG} | 19.9 ± 0.7 |
| qtRNA^{Trp}_{UAGA} | 21.4 ± 1.5 |
| qtRNA^{Trp}_{UAGA-Evo1} | 20.2 ± 1.5 |
| tRNA^{Tyr}_{GUA} | 24.4 ± 4.4 |
| qtRNA^{Tyr}_{UAGA} | 22.9 ± 3.4 |
| qtRNA^{Tyr}_{UAGA-Evo1} | 22.1 ± 3.3 |
| tRNA^{Val}_{GUC} | 19.3 ± 0.2 |
| qtRNA^{Val}_{UAGA} | 21.2 ± 0.5 |

TABLE 4
Amino acid abundance at position Y151 of sfGFP in response
to UAGA quadruplet codon translation. Mutations are indicated
for each variant using universal tRNA nomenclature⁶⁶.

| | | sfGFP | |
|---|---|---|---|
| | | Position | |
| qtRNA | Mutations | 151 (%) | LOD (%) |
| qtRNA^{Arg}_{UAGA} | — | Arg (100) | 2.14E−01 |
| qtRNA^{Arg}_{UAGA}-Evo1 | G44U | Arg (100) | 1.15E−02 |
| qtRNA^{Arg}_{UAGA}-Evo2 | C11U, U26C, | Arg (99.9), | 2.49E−04 |
| | G44U | Trp (0.1) | |
| qtRNA^{Gin}_{UAGA} | — | Gln (100) | 3.52E−02 |
| qtRNA^{Gin}_{UAGA}-Evo1 | U31C | Gln (100) | 3.56E−03 |
| qtRNA^{Gin}_{UAGA}-Evo2 | U31C, ΔU45 | Gln (100) | 6.06E−03 |
| qtRNA^{Ser}_{UAGA} | — | Ser (100) | 1.24E−02 |
| qtRNA^{Ser}_{UAGA}-Evo1 | C33A, A39C | Ser (99.95), | 5.88E−04 |
| | | Asp (0.05) | |
| qtRNA^{Ser}_{UAGA}-Evo2 | C33A, A39C, | Ser (99.96), | 1.23E−03 |
| | C53U | Asp (0.04) | |
| qtRNA^{Ser}_{UAGA}-Evo3 | U32G, C33A, | Ser (100) | 3.19E−04 |
| | A39C, A40C, | | |
| | G52A | | |
| qtRNA^{Trp}_{UAGA} | — | Trp (5.9), | 2.87E−01 |
| | | Gln (81.7), | |
| | | Tyr (12.4) | |
| qtRNA^{Trp}_{UAGA}-Evo1 | G24A, A38U, | Gln (99.99), | 3.83E−04 |
| | U72C | Tyr (0.01) | |
| qtRNA^{Tyr}_{UAGA} | — | Tyr (100) | 3.92E−03 |
| qtRNA^{Tyr}_{UAGA}-Evo1 | C33A, T34C | Tyr (100) | 9.35E−04 |

LOD: limit of detection.

TABLE 5
Strain doubling time analysis. Orthogonal qtRNA expression plasmids or an engineered
qtRNA scaffold were used to quantify cellular burden under uninduced and induced conditions.
Data represents the mean and standard deviation of 4-12 biological replicates

| | | | | | Doubling time ± standard | |
|---|---|---|---|---|---|---|
| | | | | | deviation (min) | |
| tRNA | | | | # plasmids | Uninduced | Induced |
| qtRNA^{His}_{AGGA} | | | | 1 | 19.4 ± 0.6 | 22.4 ± 0.2 |
| | qtRNA^{Gly}_{GGGG} | | | 1 | 20.0 ± 0.6 | 19.6 ± 0.4 |
| | | qtRNA^{Ser}_{UAGA-Evo3} | | 1 | 20.9 ± 3.2 | 20.1 ± 0.4 |
| | | | qtRNA^{Glu}_{CGGU} | 1 | 21.5 ± 0.5 | 21.9 ± 0.2 |
| qtRNA^{His}_{AGGA} | qtRNA^{Gly}_{GGGG} | | | 2 | 19.9 ± 0.9 | 27.2 ± 1.1 |
| | qtRNA^{Gly}_{GGGG} | qtRNA^{Ser}_{UAGA-Evo3} | | 2 | 19.5 ± 0.7 | 18.8 ± 0.5 |
| | qtRNA^{Gly}_{GGGG} | | qtRNA^{Glu}_{CGGU} | 2 | 21.1 ± 0.8 | 20.8 ± 0.8 |
| qtRNA^{His}_{AGGA} | | qtRNA^{Ser}_{UAGA-Evo3} | | 2 | 18.8 ± 0.5 | 21.5 ± 0.3 |
| qtRNA^{His}_{AGGA} | | | qtRNA^{Glu}_{CGGU} | 2 | 22.3 ± 0.5 | 23.9 ± 0.9 |
| | | qtRNA^{Ser}_{UAGA-Evo3} | qtRNA^{Glu}_{CGGU} | 2 | 22.2 ± 0.8 | 20.6 ± 0.6 |
| qtRNA^{His}_{AGGA} | | qtRNA^{Ser}_{UAGA-Evo3} | qtRNA^{Glu}_{CGGU} | 3 | 22.5 ± 0.6 | 23.9 ± 1.0 |
| qtRNA^{His}_{AGGA} | qtRNA^{Gly}_{GGGG} | qtRNA^{Ser}_{UAGA-Evo3} | | 3 | 18.8 ± 0.7 | 23.9 ± 0.6 |
| qtRNA^{His}_{AGGA} | qtRNA^{Gly}_{GGGG} | | qtRNA^{Glu}_{CGGU} | 3 | 20.9 ± 0.8 | 29.6 ± 0.6 |
| | qtRNA^{Gly}_{GGGG} | qtRNA^{Ser}_{UAGA-Evo3} | qtRNA^{Glu}_{CGGU} | 3 | 20.6 ± 0.7 | 19.7 ± 0.6 |
| qtRNA^{His}_{AGGA} | qtRNA^{Gly}_{GGGG} | qtRNA^{Ser}_{UAGA-Evo3} | qtRNA^{Glu}_{CGGU} | 4 | 20.8 ± 0.7 | 25.1 ± 0.9 |
| qtRNA^{His}_{AGGA} | qtRNA^{Gly}_{GGGG} | qtRNA^{Ser}_{UAGA-Evo3} | qtRNA^{Glu}_{CGGU} | 1 (scaffold #2) | 19.3 ± 0.5 | 19.3 ± 0.3 |

TABLE 6
Sequences of multicisronic qtRNA scaffolds. Endogenous tRNA sequences are bolded
and flanking sequences are shown in not bolded. All coordinates derive from E. coli MG1655
genome.

| | | E. coli MG1655 | |
|---|---|---|---|
| tRNA | | coordinates | |
| scaffold | Sequence (tRNA) | Start | End |
| 1 | ctctccctataatgcgactccacacagcgggggtgattagctcagctgggagagcacctcccttacaaggagggggtcggcggttcgatccc | 2520901 | 2521358 |
| | gtcatcacccaccaactactttatgtagtctccgccgtgtagcaagaaattgagaagtgggtgattagctcagctgggagagcacctccctta | | |
| | caaggagggggtcggcggttcgatcccgtcatcacccaccactttctcgccagctaaatttcttgtaaaaatgtgaagtaccgaagtgggtga | | |
| | ttagctcagctgggagagcacctcccttacaaggagggggtcggcggttcgatcccgtcatcacccaccacttcgggtcgttagctcagttgg | | |
| | tagagcagttgacttttaatcaattggtcgcaggttcgaatcctgcacgacccaccaatgtaaaaaagcgccctaaaggcgcttttt (SEQ | | |
| | ID NO: 22) | | |
| 2 | tatcaaacaaaccgaaagcaacgaaaaagtgggtcgttagctcagttggtagagcagttgacttttaatcaattggtcgcaggttcgaatcc | 780524 | 781682 |
| | tgcacgacccaccaatcgctaaggtggaagcggtagtaaaacgtgaaggataacgttgcatgagcaacggcccgaagggcgagacgaagt | | |
| | cgagtcatcctgcacgacccaccactaacatagttagttgtagtatccagcgtagtatcgggtgattagctcagctgggagagcacctccctta | | |
| | caaggagggggtcggcggttcgatcccgtcatcacccaccaccgggtcgttagctcagttggtagagcagttgacttttaatcaattggtcgc | | |
| | aggttcgaatcctgcacgacccaccagttttaacatcgaagacagatgttaagcgtgtaggataacgttgcgtcagcaacggcccgtagggc | | |
| | gagcgaagcgagtcatcctgcacgacccaccactaatgacggtgggttcggtggaagtagtttgtagtatccagcgcagtatcgggtgattag | | |
| | ctcagctgggagagcacctcccttacaaggagggggtcggcggttcgatcccgtcatcacccaccactcgggtcgttagctcagttggtaga | | |
| | gcagttgacttttaatcaattggtcgcaggttcgaatcctgcacgacccaccagttttaacatcaaactcagatgttaagcgtgaaggataac | | |
| | gttgcgccagcaacggcccgtagggcgagcgaagcgagtcatcctgcacgacccaccaatcttaaagattggccccgagtaaaaatctttca | | |
| | ggtaacacccgtatgggtcgttagctcagttggtagagcagttgacttttaatcaattggtcgcaggttcgaatcctgcacgacccaccaattt | | |
| | aaaggtggttactggtagagaacgtgaaggataacgttgcgttagcaacggcccgaagggcgagacgaagtcgagtcatcctgcacgaccc | | |
| | accatcctgaatgattaaggcagcataatcccgcaaggggtcgttagctcagttggtagagcagttgacttttaatcaattggtcgcaggttc | | |
| | gaatcctgcacgacccaccaatgtaaaaaagcgccctaaaggcgcttttt (SEQ ID NO: 23) | | |
| 3 | catgtctccatagaatgcgcgctacttgatgccgacttagctcagtaggtagagcaactgacttgtaatcagtaggtcaccagttcgattccg | 4175358 | 4175859 |
| | gtagtcggcaccatcaagtccggtggggttcccgagcggccaaagggagcagactgtaaatctgccgtcacagacttcgaaggttcgaatc | | |
| | cttcccccaccaccaatttcggccacgcgatggcgtagcccgagacgataagttcgcttaccggctcgaataaagagagcttctctcgatattc | | |
| | agtgcagaatgaaaatcaggtagccgagttccaggatgcgggcatcgtataatggctattacctcagccttccaagctgatgatgcgggttc | | |
| | gattcccgctgcccgctccaagatgtgctgatatagctcagttggtagagcgcacccttggtaagggtgaggtcggcagttcgaatctgccta | | |
| | tcagcaccacttcttttctcctccctgttttttcettct (SEQ ID NO: 24) | | |
| 4 | acgccgataaggtatcgcgaaaaaaaagatggctacgtagctcagttggttagagcacatcactcataatgatggggtcacaggttcgaat | 697163 | 696400 |
| | cccgtcgtagccaccatctttttttgcgggagtggcgaaattggtagacgcaccagatttaggttctggcgccgcaaggtgtgcgagttcaag | | |
| | tctcgcctcccgcaccattcaccagaaagcgttgtacggatggggtatcgccaagcggtaaggcaccggtttttgataccggcattccctggt | | |
| | tcgaatccaggtaccccagccatcttcttcgagtaagcggttcaccgcccggttattggggtatcgccaagcggtaaggcaccggtttttgata | | |
| | ccggcattccctggttcgaatccaggtaccccagccatcgaagaaacaatctggctacgtagctcagttggttagagcacatcactcataat | | |
| | gatggggtcacaggttcgaatcccgtcgtagccaccaaattctgaatgtatcgaatatgttcggcaaattcaaaaccaatttgttggggtatc | | |
| | gccaagcggtaaggcaccggattctgattccggcattccgaggttcgaatcctcgtaccccagccaatttattcaagacgcttaccttgtaagt | | |
| | gcacccagttggggtatcgccaagcggtaaggcaccggattctgattccggcattccgaggttcgaatcctcgtaccccagccacattaaaa | | |
| | aagctcgcttcggcgagctttt (SEQ ID NO: 25) | | |
| 5 | ccgtattatccacccccgcaacggcgctaagcgcccgtagctcagctggatagagcgctgccctccggaggcagaggtctcaggttcgaatc | 3982345 | 3982841 |
| | ctgtcgggcgcgccatttagtcccggcgcttgagctgcggtggtagtaataccgcgtaacaagatttgtagtggtggctatagctcagttggta | | |
| | gagccctggattgtgattccagttgtcgtgggttcgaatcccattagccaccccattattagaagttgtgacaatgcgaaggtggcggaattg | | |
| | gtagacgcgctagcttcaggtgttagtgtccttacggacgtgggggttcaagtcccccccctcgcaccacgactttaaagaattgaactaaaa | | |
| | attcaaaaagcagtatttcggcgagtagcgcagcttggtagcgcaactggtttgggaccagtgggtcggaggttcgaatcctctctcgccga | | |
| | ccaattttgaaccccgcttcggcggggtttttt (SEQ ID NO: 26) | | |
| 6 | agttcttcgaagcactcgtaagaggcgtgtggtgaggtggccgagaggctgaaggcgctcccctgctaagggagtatgcggtcaaaagctg | 2818675 | 2817754 |
| | catccggggttcgaatccccgcctcaccgccatttgcatccgtagctcagctggatagagtactcggctacgaaccgagcggtcggaggttc | | |
| | gaatcctcccggatgcaccatattctacgtactttcagcgatgaaggtatggaagaggtggcggtaataaccgcaggcaccagggaggataa | | |
| | cgttgctttagcaacggcccgaagggcgagccgcaaggcgagtaatcctcccggatgcaccatctcttacttgatacggctttagtagcggtat | | |
| | caaaaaatctgcagtaaagtaagtttcccgatgcatccgtagctcagctggatagagtactcggctacgaaccgagcggtcggaggttcga | | |
| | atcctcccggatgcaccatctcttacttgatatggctttagtagcggtatcaatatcagcagtaaaataaatttcccgatgcatccgtagctcag | | |
| | ctggatagagtactcggctacgaaccgagcggtcggaggttcgaatcctcccggatgcaccatattctccgaaatcttcagcaatgaaggta | | |
| | tcgaagaggtagcggaattaaccgcaggcactagggatgataacgttgctttagcaacggcccgaagggcgagccgcaaggcgagtaatcc | | |
| | tcccggatgcaccatctcttaattgatatggctttagtagcggtatcaatatcagcagtagaataagttttcccgatgcatccgtagttcagctg | | |
| | gatagagtactcggctacgaaccgagcggtcggaggttcgaatcctcccggatgcaccatattctccgtaaccttcagcaatgaaggta | | |
| | (SEQ ID NO: 27) | | |

TABLE 7
Sequences of multicisronic qtRNA scaffolds. All qtRNAs are visualized as bold and
capitalized, with their anticodons underlined. Flanking sequences were included in vector design
to ensure efficient qtRNA maturation. qtRNA order in each scaffold is as follows:
qtRNAGlyGGGG, qtRNASerUAGA-Evo3, qtRNAGluCGGU, then qtRNAHisAGGA.

| tRNA | |
|---|---|
| scaffold | Sequence (tRNA) |
| 1 | cctataatgcgactccacacagcggGCGGGCGTAGTTCAATGGTAGAACGAGAGCTTCCCCAAGCTCTATACGAGGGTTCGATTCCCTTCGCCC |
| | GCTCCAactactttatgtagtctccgccgtgtagcaagaaattgagaagtGGAGAGATGCCGGAGCGGCTGAACGGACCGGGATTCTAACCCCGGAG |
| | TAGGGACAACTCTACCGGGGGTTCAAATCCCCCTCTCTCCGCCActttctcgccagctaaatttcttgtaaaaatgtgaagtaccgaagtGTCCCCTTCGT |
| | CTAGAGGCCCAGGACACCGCCCTACCGACGGCGGTAACAGGGGTTCGAATCCCCTAGGGGACGCCActtGTGGCTATAGCTCAGTTG |
| | GTAGAGCCCTGGATTTCCTATTCCAGTTGTCGTGGGTTCGAATCCCATTAGCCACCCCAatgtaaaaaagcgccctaaaggcgc (SEQ ID NO: |
| | 28) |
| 2 | tagtttgtagtatccagcgcagtatcGCGGGCGTAGTTCAATGGTAGAACGAGAGCTTCCCCAAGCTCTATACGAGGGTTCGATTCCCTTCGCCC |
| | GCTCCActcGGAGAGATGCCGGAGCGGCTGAACGGACCGGGATTCTAACCCCGGAGTAGGGACAACTCTACCGGGGGTTCAAATCCC |
| | CCTCTCTCCGCCAgttttaacatcaaactcagatgttaagcgtgaaggataacgttgcgccagcaacggcccgtagggcgagcgaagcgagtcatcctgcacgacccacca |
| | atcttaaagattggccccgagtaaaaatctttcaggtaacacccgtatGTCCCCTTCGTCTAGAGGCCCAGGACACCGCCCTACCGACGGCGGTAACAG |
| | GGGTTCGAATCCCCTAGGGGACGCCAatttaaaggtggttactggtagagaacgtgaaggataacgttgcgttagcaacggcccgaagggcgagacgaagtcgag |
| | tcatcctgcacgacccaccatcctgaatgattaaggcagcataatcccgcaagGTGGCTATAGCTCAGTTGGTAGAGCCCTGGATTTCCTATTCCAGTTGT |
| | CGTGGGTTCGAATCCCATTAGCCACCCCAatgtaaaaaagcgccctaaaggcgc (SEQ ID NO: 29) |
| 3 | tctccatagaatgcgcgctacttgatGCGGGCGTAGTTCAATGGTAGAACGAGAGCTTCCCCAAGCTCTATACGAGGGTTCGATTCCCTTCGCCC |
| | GCTCCAtcaagtccGGAGAGATGCCGGAGCGGCTGAACGGACCGGGATTCTAACCCCGGAGTAGGGACAACTCTACCGGGGGTTCAAA |
| | TCCCCCTCTCTCCGCCAatttcggccacgcgatggcgtagcccgagacgataagttcgcttaccggctcgaataaagagagcttctctcgatattcagtgcagaatgaaaat |
| | caggtagccgagttccaggatGTCCCCTTCGTCTAGAGGCCCAGGACACCGCCCTACCGACGGCGGTAACAGGGGTTCGAATCCCCTAGGGG |
| | ACGCCAagatgtGTGGCTATAGCTCAGTTGGTAGAGCCCTGGATTTCCTATTCCAGTTGTCGTGGGTTCGAATCCCATTAGCCACCCCAct |
| | tcttttctcctccctgttttttc (SEQ ID NO: 30) |
| 4 | ttatccacccccgcaacggcgctaaGCGGGCGTAGTTCAATGGTAGAACGAGAGCTTCCCCAAGCTCTATACGAGGGTTCGATTCCCTTCGCCC |
| | GCTCCAtctttttttGGAGAGATGCCGGAGCGGCTGAACGGACCGGGATTCTAACCCCGGAGTAGGGACAACTCTACCGGGGGTTCAAA |
| | TCCCCCTCTCTCCGCCAttcaccagaaagcgttgtacggaGTCCCCTTCGTCTAGAGGCCCAGGACACCGCCCTACCGACGGCGGTAACAGGGG |
| | TTCGAATCCCCTAGGGGACGCCAtcttcttcgagtaagcggttcaccgcccggttatGTGGCTATAGCTCAGTTGGTAGAGCCCTGGATTTCCTATT |
| | CCAGTTGTCGTGGGTTCGAATCCCATTAGCCACCCCAtcgaagaaacaatctggctacgtag (SEQ ID NO: 31) |
| 5 | ttatccacccccgcaacggcgctaaGCGGGCGTAGTTCAATGGTAGAACGAGAGCTTCCCCAAGCTCTATACGAGGGTTCGATTCCCTTCGCCC |
| | GCTCCAtttagtcccggcgcttgagctgcggtggtagtaataccgcgtaacaagatttgtagtgGGAGAGATGCCGGAGCGGCTGAACGGACCGGGATTCT |
| | AACCCCGGAGTAGGGACAACTCTACCGGGGGTTCAAATCCCCCTCTCTCCGCCAttattagaagttgtgacaatGTCCCCTTCGTCTAGAGGCC |
| | CAGGACACCGCCCTACCGACGGCGGTAACAGGGGTTCGAATCCCCTAGGGGACGCCAcgactttaaagaattgaactaaaaattcaaaaagcagta |
| | tttGTGGCTATAGCTCAGTTGGTAGAGCCCTGGATTTCCTATTCCAGTTGTCGTGGGTTCGAATCCCATTAGCCACCCCAattttgaaccccgc |
| | ttcggcggggt (SEQ ID NO: 32) |
| 6 | ttcgaagcactcgtaagaggcgtgtGCGGGCGTAGTTCAATGGTAGAACGAGAGCTTCCCCAAGCTCTATACGAGGGTTCGATTCCCTTCGCCC |
| | GCTCCAtttGGAGAGATGCCGGAGCGGCTGAACGGACCGGGATTCTAACCCCGGAGTAGGGACAACTCTACCGGGGGTTCAAATCCC |
| | CCTCTCTCCGCCAtattctacgtactttcagcgatgaaggtatggaagaggtggcggtaataaccgcaggcaccagggaggataacgttgctttagcaacggcccgaaggg |
| | cgagccgcaaggcgagtaatcctcccggatgcaccatctcttacttgatacggctttagtagcggtatcaaaaaatctgcagtaaagtaagtttcccgatGTCCCCTTCGTCT |
| | AGAGGCCCAGGACACCGCCCTACCGACGGCGGTAACAGGGGTTCGAATCCCCTAGGGGACGCCAtctcttacttgatatggctttagtagcggta |
| | tcaatatcagcagtaaaataaatttcccgatGTGGCTATAGCTCAGTTGGTAGAGCCCTGGATTTCCTATTCCAGTTGTCGTGGGTTCGAATCCCA |
| | TTAGCCACCCCAtattctccgaaatcttcagcaatgaagg (SEQ ID NO: 33) |

TABLE 8
Summary LC-MS/MS results.

| | | Reporter | Amino acid | |
|---|---|---|---|---|
| | | (Position > | abundance at | |
| | | Quadruplet | respective | |
| Figure | qtRNA | Codon) | position(s) (%) | Fragmentation sequence |
| FIG. 8a | qtRNA^{Gly}_{GGGG} | sfGFP | Gly (100) | K.LEYNFN(+.98)SHNVG(sub Y)ITADK.Q |
| | | (Y151 > GGGG) | | K.LEYNFNSHNVG(sub Y)ITADKQK.N |
| | | | | K.LEYNFNSHNVG(sub Y)ITADK.Q |
| | | | | K.LEYNFN(+.98)SHNVG(sub Y)ITADKQK.N |
| | | | | (SEQ ID NOS: 34-36) |
| FIG. 8b | qtRNA^{His}_{AGGA} | sfGFP | His (100) | K.LEYN(+.98)FNSHNVH(sub Y)ITADK.Q |
| | | (Y151 > AGGA) | | K.LEYNFN(+.98)SHNVH(sub Y)ITADKQK.N |
| | | | | (SEQ ID NOS: 37-38) |
| FIG. 8c | qtRNA^{Thr}_{ACCA} | sfGFP | Thr (100) | K.LEYNFNSHNVT(sub Y)ITADK.Q |
| | | (Y151 > ACCA) | | K.LEYNFNSHNVT(sub Y)ITADKQK.N |
| | | | | (SEQ ID NOS: 39-40) |
| FIG. 8d | qtRNA^{Glu}_{CGGU} | sfGFP | Glu (98), Arg | K.LEYNFNSHNVR(sub Y).I |
| | | (Y151 > CGGU) | (2) | K.LEYNFNSHNVE(sub Y)ITADK.Q |
| | | | | K.LEYNFNSHNVE(sub Y)ITADKQK.N |
| | | | | K.LEYNFN(+.98)SHNVR(sub Y).I |
| | | | | (SEQ ID NOS: 41-44) |
| FIG. 8e | qtRNA^{Tyr}_{UAGA} | sfGFP | Tyr (100) | K.LEYNFNSHNVYITADK.Q |
| | | (Y151>UAGA) | | K.LEYNFNSHNVYITADKQK.N |
| | | | | (SEQ ID NOS: 45-46) |
| FIG. 12a | qtRNA^{Arg}_{UAGA} | sfGFP | Arg (100) | Y.NFNSHNVR(sub Y)ITADKQKNGIKANF.K |
| | | (Y151 > UAGA) | | (SEQ ID NO: 47) |
| FIG. 12b | qtRNA^{Arg}_{UAGA}- | sfGFP | Arg (100) | Y.NFNSHNVR(sub Y)ITADKQKNGIKANF.K |
| | Evo1 | (Y151 > UAGA) | | (SEQ ID NO: 48) |
| FIG. 12c | qtRNA^{Arg}_{UAGA}- | sfGFP | Arg (99.9), Trp | K.LEYNFNSHNVR(sub Y).I |
| | Evo2 | (Y151 > UAGA) | (0.1) | K.LEYNFNSHNVW(sub Y).I |
| | | | | (SEQ ID NOS: 49-50) |
| FIG. 12d | qtRNA^{Gln}_{UAGA} | sfGFP | Gln (100) | K.LEYNFNSHNVQ(sub Y)ITADK.Q |
| | | (Y151 > UAGA) | | K.LEYNFNSHNVQ(sub Y)ITADKQK.N |
| | | | | (SEQ ID NOS: 51-52) |
| FIG. 12e | qtRNA^{Gln}_{UAGA}- | sfGFP | Gln (100) | K.LEYNFNSHNVQ(sub Y)ITADK.Q |
| | Evo1 | (Y151 > UAGA) | | K.LEYNFNSHNVQ(sub Y)ITADKQK.N |
| | | | | (SEQ ID NOS: 53-54) |
| FIG. 12f | qtRNA^{Gln}_{UAGA}- | sfGFP | Gln (100) | K.LEYNFNSHNVQ(sub Y)ITADK.Q |
| | Evo2 | (Y151 > UAGA) | | K.LEYNFNSHNVQ(sub Y)ITADKQK.N |
| | | | | (SEQ ID NOS: 55-56) |
| FIG. 12g | qtRNA^{Ser}_{UAGA} | sfGFP | Ser (100) | K.LEYNFNSHNVS(sub Y)ITADK.Q |
| | | (Y151 > UAGA) | | K.LEYNFNSHNVS(sub Y)ITADKQK.N |
| | | | | (SEQ ID NOS: 57-58) |
| FIG. 12h | qtRNA^{Ser}_{UAGA}- | sfGFP | Ser (99.95), | K.LEYNFNSHNVD(sub Y)ITADK.Q |
| | Evo1 | (Y151 > UAGA) | Asp (0.05) | K.LEYNFNSHNVS(sub Y)ITADK.Q |
| | | | | K.LEYNFNSHNVS(sub Y)ITADKQK.N |
| | | | | (SEQ ID NOS: 59-61) |
| FIG. 12i | qtRNA^{Ser}_{UAGA}- | sfGFP | Ser (99.96), | K.LEYNFNSHNVD(sub Y)ITADK.Q |
| | Evo2 | (Y151 > UAGA) | Asp (0.04) | K.LEYNFNSHNVS(sub Y)ITADKQK.N |
| | | | | K.LEYNFNSHNVS(sub Y)ITADK.Q |
| FIG. 12j | qtRNA^{Ser}_{UAGA}- | sfGFP | Ser (100) | (SEQ ID NOS: 62-64) |
| | Evo3 | (Y151 > UAGA) | | K.LEYNFNSHNVS(sub Y)ITADK.Q |
| | | | | (SEQ ID NO: 65) |
| FIG. 12k | qtRNA^{Trp}_{UAGA} | sfGFP | Trp (5.9), Gln | K.LEYNFNSHNVW(sub Y)ITADK.Q |
| | | (Y151 > UAGA) | (81.7), Tyr | K.LEYNFNSHNVYITADKQK.N |
| | | | (12.4) | K.LEYNFNSHNVQ(sub Y)ITADKQK.N |
| | | | | (SEQ ID NOS: 66-68) |
| FIG. 121 | qtRNA^{Trp}_{UAGA}- | sfGFP | Gln (99.99), | K.LEYNFNSHNVQ(sub Y)ITADK.Q |
| | Evo1 | (Y151 > UAGA) | Tyr (0.01) | K.LEYNFNSHNVQ(sub Y)ITADKQK.N |
| | | | | K.LEYNFNSHNVYITADKQK.N |
| | | | | (SEQ ID NOS: 69-71) |
| FIG. 12m | qtRNA^{Tyr}_{UAGA} | sfGFP | Tyr (100) | K.LEYNFNSHNVYITADK.Q |
| | | (Y151 > UAGA) | | K.LEYNFNSHNVYITADKQK.N |
| | | | | (SEQ ID NOS: 72-73) |
| FIG. 12n | qtRNA^{Tyr}_{UAGA}- | sfGFP | Tyr (100) | K.LEYNFNSHNVYITADKQK.N |
| | Evo1 | (Y151 > UAGA) | | (SEQ ID NOS: 74) |
| FIG. 15a | qtRNASer UAGA- | sfGFP-(6xUAGA- | Ser (100) | K.TSHLNSSSSSSHASVSKGEEDNM(+15.99)AIIK.E |
| | Evo3 | linker)-mCherry | | K.TSHLNSSSSSSHASVSKGEEDNM(+15.99)AIIKEF |
| | | | | M(+15.99)R.F |
| | | | | (SEQ ID NOS: 75-76) |
| FIG. 15b | qtRNA^{Tyr}_{UAGA}- | sfGFP-(6xUAGA- | Tyr (100) | K.TSHLNYYYYYYHASVSKGEEDNM(+15.99)AIIK.E |
| | Evo1 | linker)-mCherry | | (SEQ ID NO: 77) |
| FIG. 15c | qtRNA^{Gln}_{UAGA}- | sfGFP-(5xUAGA- | Gln (100) | K.TSHLNQQQQQHASVSKGEEDNM(+15.99)AIIK.E |
| | Evo2 | linker)-mCherry | | K.TSHLNQQQQQHASVSKGEEDNM(+15.99)AIIKEF |
| | | | | M(+15.99)R.F |
| | | | | (SEQ ID NOS: 78-79) |
| FIG. 16a | qtRNA^{His}_{AGGA} | sfGFP | His (100) | K.LEYNFNSHNVYITADKQK.N |
| | | (H148 > AGGA) | | K.LEYNFNSHNVYITADK.Q |
| | | | | (SEQ ID NOS: 80-81) |
| FIG. 16b | qtRNA^{Gly}_{GGGG} | sfGFP | Gly (100) | R.HNVEDGSVQLADH.Y |
| | | (G174 > GGGG) | | K.IRHNVEDGSVQLADHYQQNTPIGDGPVLLPDNHY |
| | | | | LSTQSVLSK.D |
| | | | | (SEQ ID NOS: 82-83) |
| FIG. 16c | qtRNA^{Ser}_{UAGA}- | sfGFP | Ser (100) | N.TPIGDGPVLLPDNHYLSTQSVLSKDPNEKR.D |
| | Evo3 | (S202 > UAGA) | | N.TPIGDGPVLLPDNHYLSTQSVLSK.D |
| | | | | (SEQ ID NOS: 84-85) |
| FIG. 16d | qtRNA_{Glu}^{CGGU} | sfGFP | Glu (100) | K.DPNEKRDHM(+15.99)VLLEFVTAAGITHGM |
| | | (E213 > CGGU) | | (+15.99)DELYK.G |
| | | | | R.HNVEDGSVQLADHYQQNTPIGDGPVLLPDNHYLS |
| | | | | TQSVLSKDPNEK.R |
| | | | | (SEQ ID NOS: 86-87) |
| FIG. 19a | qtRNA scaffold | sfGFP | H148: His | K.LEYNFN(+.98)SHNVYITADKQK.N |
| | 2 (Gly-GGGG, | (H148 > AGGA, | (100); G174: | (SEQ ID NO: 88) |
| | Ser-UAGA- | G174 > GGGG, | Gly (100); | K.IRHNVEDGSVQLADHYQQNTPIGDGPVLLPDNHY |
| | Evo3, Glu- | S202 > UAGA) | S202: Ser (100) | LSTQSVLSK.D |
| | CGGU, His- | | | (SEQ ID NO: 89) |
| | AGGA) | | | |
| FIG. 19b | qtRNA scaffold | sfGFP(H148> | H148: His | K.LEYNFNSHNVYITADK.Q |
| | 2 (Gly-GGGG, | AGGA, G174 > GGGG, | (100); G174: | (SEQ ID NO: 90) |
| | Ser-UAGA- | E213 > CGGU) | Gly (100); | R.HNVEDGSVQLADHYQQNTPIGDGPVLLPDNHYLS |
| | Evo3, Glu- | | E213: Glu (100) | TQSVLSK.D (SEQ ID NO: 91) |
| | CGGU, His- | | | K.DPNEKRDHMVLLEFVTAAGITHGMDELYK.G |
| | AGGA) | | | (SEQ ID NO: 92) |
| FIG. 19c | qtRNA scaffold | sfGFP | H148: His (86), | K.LEYNFNSR(sub H).N |
| | 2 (Gly-GGGG, | (H148 > AGGA, | Arg (14); S202: | K.LEYNFNSHNVYITADK.Q |
| | Ser-UAGA- | S202 > UAGA, | Ser (100); | R.HNVEDGSVQLADHYQQNTPIGDGPVLLPDNHYLS |
| | Evo3, Glu- | E213 > CGGU) | E213: Glu (100) | TQSVLSK.D |
| | CGGU, His- | | | K.DPNEKRDHM(+15.99)VLLEFVTAAGITHGM |
| | AGGA) | | | (+15.99)DELYK.G |
| | | | | (SEQ ID NOS: 93-96) |
| FIG. 19d | qtRNA scaffold | sfGFP | G174: Gly | K.IRHNVEDGSVQLADHYQQNTPIGDGPVLLPDNHY |
| | 2 (Gly-GGGG, | (G174 > GGGG, | (100); S202: | LSTQSVLSK.D |
| | Ser-UAGA- | S202 > UAGA, | Ser (100); | (SEQ ID NO: 97) |
| | Evo3, Glu- | E213 > CGGU) | E213: Glu (100) | K.DPNEKRDHM(+15.99)VLLEFVTAAGITHGM |
| | CGGU, His- | | | (+15.99)DELYK.G |
| | AGGA) | | | (SEQ ID NO: 98) |
| FIG. 21 | qtRNA scaffold | sfGFP | H148: His | K.LEYNFNSHNVYITADKQK.N |
| | 2 (Gly-GGGG, | (H148 > AGGA, | (100); G174: | K.LEYNFN(+.98)SHNVYITADK.Q |
| | Ser-UAGA- | G174 > GGGG, | Gly (100); | (SEQ ID NOS: 99-100) |
| | Evo3, Glu- | S202 > UAGA, | S202: Ser | R.HNVEDGSVQLADHYQQNTPIGDGPVLLPDNHYLS |
| | CGGU, His- | E213 > CGGU) | (100); E213: | TQSVLSKDPNEKR.D |
| | AGGA) | | Glu (100) | (SEQ ID NO: 101) |

Various modifications and variations of the described methods, pharmaceutical compositions, and kits of the invention will be apparent to those skilled in the art without departing from the scope and spirit of the invention. Although the invention has been described in connection with specific embodiments, it will be understood that it is capable of further modifications and that the invention as claimed should not be unduly limited to such specific embodiments. Indeed, various modifications of the described modes for carrying out the invention that are obvious to those skilled in the art are intended to be within the scope of the invention. This application is intended to cover any variations, uses, or adaptations of the invention following, in general, the principles of the invention and including such departures from the present disclosure come within known customary practice within the art to which the invention pertains and may be applied to the essential features herein before set forth.

What is claimed is:

1. An engineered multicistronic expression construct comprising at least 3-4 quadruplet-decoding tRNA (qtRNA) encoding regions.

2. The engineered expression construct of claim 1, wherein the expression construct is derived from an endogenous multicistronic tRNA operon.

3. The engineered expression construct of claim 2, wherein the endogenous multicistronic tRNA is from a bacterium.

4. The engineered expression construct of claim 3, wherein the bacterium is E. coli.

5. The engineered expression construct of any of claims 1 to 4, wherein at least one of the qtRNAs are non-canonical amino acid (ncAA) qtRNAs.

6. The engineered expression construct of any of claims 1 to 5, further comprising an RNA polymerase III promoter.

7. A vector comprising the engineered expression construct of any one of claims 1 to 6.

8. The vector of claim 7, wherein the vector is a prokaryotic expression vector.

9. The vector of claim 7, wherein the vector is a eukaryotic expression vector.

10. The vector of claim 9, wherein the eukaryotic expression vector is a mammalian, insect, or yeast expression vector.

11. A cell comprising the engineered expression construct of any of claims 1 to 6, or the vector of any one of claims 7 to 10.

12. The cell of claim 11, wherein the scaffold is integrated into the genome of the cell or capable of autonomous replication in the cell.

13. A method of decoding multiple orthogonal quadruplet codons in a cell comprising expressing in the cell the engineered expression construct of claims 1 to 6, or the vector of any one of claims 7 to 10.

14. A method of increasing the efficiency of a quadruplet-decoding tRNA (qtRNA) comprising performing phage-assisted continuous evolution (PACE) on a qtRNA, wherein the PACE system comprises: i) an M13 selection phage (SP) encoding the qtRNA in place of gIII, ii) an accessory plasmid (AP) encoding gIII comprising one or more quadruplet codons that are required to be decoded by the qtRNA in order to produce functional pIII, and iii) an inducible mutagenesis plasmid (MP) encoding one or more mutagenic proteins capable of increasing the rate of evolution.

15. The method of claim 14, wherein the AP comprises 1 to 4 quadruplet codons.

16. The method of claim 14 or 15, wherein gIII comprises one or more quadruplet codons at a permissive residue.

17. The method of any of claims 14 to 16, wherein gIII comprises one or more quadruplet codons at a non-permissive residue.

18. The method of any of claims 14 to 17, wherein MP is the MP6 plasmid.

19. The method of any of claims 14 to 18, wherein the qtRNA is a non-canonical amino acid (ncAA) qtRNA and the PACE system further comprises an aminoacyl-tRNA synthetase for the ncAA.

20. The method of any of claims 14 to 19, wherein the starting qtRNA is selected based on decoding of a quadruplet codon in a reporter gene and inserting an amino acid required for translating a functional reporter gene.

21. The method of any of claims 14 to 20, wherein the starting qtRNA comprises a quadruplet anticodon and an amino acid specific tRNA scaffold.

22. The method of any of claims 14 to 21, further comprising determining decoding activity of evolved qtRNAs by expressing an evolved qtRNA in a cellular system comprising a nucleotide sequence encoding a selection marker comprising one or more quadruplet codons, wherein the selection marker is functional only if the one or more quadruplet codons are decoded by the qtRNA; and comparing expression of the selection marker with a cellular system comprising a nucleotide sequence encoding the selection marker having a wild-type codon sequence.

23. The method of claim 22, wherein the selection marker comprises 1 to 6 quadruplet codons.

24. The method of claim 22 or 23, wherein the selection marker is a luminescent, fluorescent, or enzymatic selection marker.

25. The method of claim 24, wherein the selection marker comprises a luciferase (LuxAB), sfGFP, or β-galactosidase (LacZ) selection marker.

26. The method of claim 24, wherein the selection marker is a dual fluorescent selection marker comprising adjacent quadruplet codons in a linker sequence between the dual fluorescent markers.

27. The method of any of claims 22 to 26, wherein the selection marker comprises one or more quadruplet codons at a permissive residue.

28. The method of any of claims 22 to 27, wherein the selection marker comprises one or more quadruplet codons at a non-permissive residue.

29. The method of any of claims 22 to 28, wherein the cellular system is a prokaryotic or eukaryotic cellular system.

30. The method of claim 29, wherein the eukaryotic cellular system consists of yeast cells.

31. The method of claim 29, wherein the eukaryotic cellular system consists of insect cells.

32. The method of claim 29, wherein the eukaryotic cellular system consists of mammalian cells.

33. The method of any of claims 22 to 32, wherein the cellular system is modified to decrease or abolish expression of a cellular release factor.

34. A qtRNA selected from Table 4.
